(19) United States
(12) Patent Application Publication
(10) Pub. No.: US 2002/0198179 A1
(43) Pub. Date: Dec. 26, 2002

(54) Selective estrogen receptor modulators in combination with estrogens

(75) Inventor: Fernand Labrie, Sainte-foy (CA)

Correspondence Address: OSTROLENK FABER GERB & SOFFEN, 1180 AVENUE OF THE AMERICAS, NEW YORK, NY, 100368403

(73) Assignee: Endorecherche, Inc.

(21) Appl. No.: 10/052,803

(22) Filed: Nov. 7, 2001

Related U.S. Application Data
(63) Continuation of application No. 09/771,180, filed on Jan. 26, 2001.
(60) Provisional application No. 60/178,601, filed on Jan. 28, 2000.

(51) Int. Cl. A61K 31/66; A61K 31/56

(52) U.S. Cl. 514/102; 514/171

(57) ABSTRACT

Novel methods for reduction or elimination the incidence of hot flashes and menopausal symptoms, while decreasing the risk of acquiring breast or endometrial cancer and furthermore treating and/or inhibiting the development of osteoporosis, hypercholesterolemia, hyperlipidemia, atherosclerosis, hypertension, insulin resistance, diabetes, loss of muscle mass, obesity, irregular menstruation, Alzheimer's disease, or vaginal dryness in susceptible warm-blooded animals including humans involving administration of selective estrogen receptor modulator, particularly compounds having the general structure:

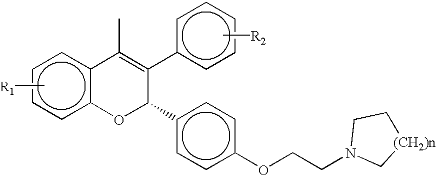

and an amount of an estrogen or mixed estrogenic/androgenic compound. Further administration of bisphosphonates, or sex steroid precursor is specifically disclosed for the medical treatment and/or inhibition of development of some of these above-mentioned diseases. Pharmaceutical compositions for delivery of active ingredient(s) and kit(s) useful to the invention are also disclosed.

### RELATED APPLICATIONS

This application is a divisional of U.S. patent application Ser. No. 09/771,180, filed Jan. 26, 2001, which is based upon and claims priority of U.S. Provisional Application No. 60/178,601, filed Jan. 28, 2000, which is hereby incorporated by reference.

### FIELD OF THE INVENTION

The present invention relates to novel combinations of physiologically active compounds. In particular, the combination includes a selective estrogen receptor modulator (SERM) in combination with an estrogen. In some embodiments, the combination includes a selective estrogen receptor modulator (SERM), an estrogen and a precursor of sex steroids or an androgenic compound. The invention also provides kits and pharmaceutical compositions for practicing the foregoing combination. Administering the foregoing combination to patients to reduce or eliminate the incidence of hot flashes, vasomotor symptoms, vaginal dryness or other menopausal symptoms. The risk of acquiring breast cancer and/or endometrial cancer is believed to be reduced for patients receiving this combination therapy. Methods of treating or reducing the likelihood of acquiring osteoporosis, hypercholesterolemia, hyperlipidemia, atherosclerosis, hypertension, Alzheimer's disease, insomnia, cardiovascular diseases, insulin resistance, diabetes, and obesity (especially abdominal obesity) is also provided.

### BACKGROUND

It is known that a large number of diseases, conditions and undesirable symptoms respond favorably to administering exogenous sex steroids, or precursors thereof. For example, estrogens are believed to decrease the rate of bone loss while androgens have been shown to build bone mass by stimulating bone formation. Hormone replacement therapy (e.g., administration of estrogens) may be used for the treatment of menopausal symptoms. Progestins are frequently used to counteract the endometrial proliferation and the risk of endometrial cancer induced by estrogens. Use of estrogens, androgenic compounds and/or progestins for treatment, or for prophylactic purposes, for a wide variety of symptoms and disorders suffer from a number of weaknesses. Treatment of females with androgenic compounds may have the undesirable side effect of causing certain masculinizing side effects. Also, administering sex steroids to patients may increase the patient's risk of acquiring certain diseases. Female breast cancer, for example, is exacerbated by estrogenic activity. Prostatic cancer and benign prostatic hyperplasia are both exacerbated by androgenic activity. More effective hormonal therapies, and reduction of side effects and risk are needed.

The combination therapies of the present invention, and the pharmaceutical compositions and kits that may be used in those therapies, are believed to address these needs.

### SUMMARY OF THE INVENTION

It is an object of the present invention to provide a method of treating or reducing the incidence or risk of acquiring hot flashes, vasomotor symptoms, osteoporosis, cardiovascular diseases, hypercholesterolemia, hyperlipidemia, atherosclerosis, hypertension, insulin resistance, diabetes, obesity (especially abdominal obesity), irregular menstruation and vaginal dryness.

It is another object to provide methods of treating or reducing the risk of acquiring the above-indicated diseases, while minimizing undesirable side-effects.

It is another object to provide kits and pharmaceutical compositions suitable for use in the above methods.

In one embodiment, the invention provides a method of reducing or eliminating the incidence of menopausal symptoms, said method comprising administering to patient in need of said elimination or reduction, a therapeutically effective amount of an estrogen or prodrug thereof in association with administering to said patient a therapeutically effective amount of a selective estrogen receptor modulator or prodrug thereof, said modulator being a different compound from said estrogen.

In another embodiment the invention provides a method of treating or reducing the risk of acquiring a condition selected from the group consisting of osteoporosis, hypercholesterolemia, hyperlipidemia, atherosclerosis, hypertension, Alzheimer's disease, insulin resistance, diabetes, loss of muscle mass, obesity, vaginal bleeding induced by hormone replacement therapy, and breast tenderness induced by hormone replacement therapy, said method comprising administering to patient in need of said elimination or reduction, a therapeutically effective amount of an estrogen or prodrug thereof in association with administering to said patient a therapeutically effective amount of a selective estrogen receptor modulator or prodrug thereof, said modulator being a different compound from said estrogen.

In another embodiment the invention provides a pharmaceutical composition comprising:

a) a pharmaceutically acceptable excipient, diluent or carrier;

b) a therapeutically effective amount of at least one estrogen or prodrug thereof; and

c) a therapeutically effective amount of at least one selective estrogen receptor modulator or prodrug thereof, wherein said modulator is a different compound from said estrogen.

In another embodiment the invention provides a kit comprising a first container containing a pharmaceutical formulation comprising a therapeutically effective amount of at least one estrogen or a prodrug thereof; and said kit further comprising a second container containing a pharmaceutical formulation comprising a therapeutically effective amount of at least one selective estrogen receptor modulator or prodrug thereof.

In one embodiment, the invention pertains to a method of treating or reducing the risk of acquiring osteoporosis comprising administering to said patient a therapeutically effective amount of an estrogen, and further comprising administering to said patient a therapeutically effective amount of a selective estrogen receptor modulator (SERM) as part of a combination therapy.

In another embodiment, the invention provides a method of treating or reducing the risk of acquiring cardiovascular diseases comprising administering to said patient a therapeutically effective amount of an estrogen, and further comprising administering to said patient a therapeutically effective amount of a selective estrogen receptor modulator (SERM) as part of a combination therapy.

In another embodiment, the invention provides a method of treating or reducing the risk of acquiring hypercholesterolemia comprising administering to said patient a therapeutically effective amount of an estrogen, and further comprising administering to said patient a therapeutically effective amount of a selective estrogen receptor modulator (SERM) as part of a combination therapy.

In another embodiment, the invention provides a method of treating or reducing the risk of acquiring hyperlipidemia comprising administering to said patient a therapeutically effective amount of an estrogen, and further comprising administering to said patient a therapeutically effective amount of a selective estrogen receptor modulator (SERM) as part of a combination therapy.

In another embodiment, the invention provides a method of treating or reducing the risk of acquiring atherosclerosis comprising administering to said patient a therapeutically effective amount of an estrogen, and further comprising administering to said patient a therapeutically effective amount of a selective estrogen receptor modulator (SERM) as part of a combination therapy.

In another embodiment, the invention provides a method of treating or reducing the risk of acquiring hypertension, comprising administering to said patient a therapeutically effective amount of an estrogen, and further comprising administering to said patient a therapeutically effective amount of a selective estrogen receptor modulator (SERM) as part of a combination therapy.

In another embodiment, the invention provides a method of treating or reducing the risk of developing insomnia, comprising administering to said patient a therapeutically effective amount of an estrogen, and further comprising administering to said patient a therapeutically effective amount of a selective estrogen receptor modulator (SERM) as part of a combination therapy.

In another embodiment, the invention provides a method of treating or reducing the risk of developing loss of cognitive functions, comprising administering to said patient a therapeutically effective amount of an estrogen, and further comprising administering to said patient a therapeutically effective amount of a selective estrogen receptor modulator (SERM) as part of a combination therapy.

In another embodiment, the invention provides a method of treating or reducing the risk of acquiring Alzheimer's disease, comprising administering to said patient a therapeutically effective amount of an estrogen, and further comprising administering to said patient a therapeutically effective amount of a selective estrogen receptor modulator (SERM) as part of a combination therapy.

In another embodiment, the invention provides a method of treating or reducing the risk of acquiring diabetes, comprising administering to said patient a therapeutically effective amount of an estrogen, and further comprising administering to said patient a therapeutically effective amount of a selective estrogen receptor modulator (SERM) as part of a combination therapy.

In another embodiment, the invention provides a method of treating or reducing the risk of developing menopausal symptoms, comprising administering to said patient a therapeutically effective amount of an estrogen, and further comprising administering to said patient a therapeutically effective amount of a selective estrogen receptor modulator (SERM) as part of a combination therapy.

In another embodiment, the invention provides a method of treating or reducing the risk of acquiring obesity (especially abdominal obesity), comprising administering to said patient a therapeutically effective amount of an estrogen, and further comprising administering to said patient a therapeutically effective amount selective estrogen receptor modulator (SERM) as part of a combination therapy.

In another embodiment, the invention provides a method of treating or reducing the risk of developing menopausal symptoms, comprising administering to said patient a therapeutically effective amount of an estrogen, and further comprising administering to said patient a therapeutically effective amount selective estrogen receptor modulator (SERM) as part of a combination therapy.

In another embodiment, the invention provides a method of treating or reducing the risk of developing breast tenderness induced by hormone replacement therapy, comprising administering to said patient a therapeutically effective amount of an estrogen, and further comprising administering to said patient a therapeutically effective amount selective estrogen receptor modulator (SERM) as part of a combination therapy.

In another embodiment, the invention provides a method of treating or reducing the risk of developing vaginal bleeding induced by hormone replacement therapy, comprising administering to said patient a therapeutically effective amount of an estrogen, and further comprising administering to said patient a therapeutically effective amount selective estrogen receptor modulator (SERM) as part of a combination therapy.

In another embodiment, the invention pertains to a method of treating or reducing the incidence of osteoporosis increasing levels of a sex steroid precursor selected from the group consisting of dehydroepiandrosterone (DHEA), dehydroepiandrosterone-sulfate (DHEA-S), androstenedione and androst-5-ene-3β,17β-diol (5-diol), in a patient in need of said treatment or said reduction, and further comprising administering to said patient a therapeutically effective amount of a selective estrogen receptor modulator (SERM) and a therapeutically effective amount of an estrogen as part of a combination therapy.

In another embodiment, the invention pertains to a method of treating or reducing the incidence of hot flashes and sweat by increasing levels of a sex steroid precursor selected from the group consisting of dehydroepiandrosterone (DHEA), dehydroepiandrosterone-sulfate (DHEA-S) and androst-5-ene-3β,17β-diol (5-diol), in a patient in need of said treatment or said reduction, and further comprising administering to said patient a therapeutically effective amount of a selective estrogen receptor modulator (SERM) and a therapeutically effective amount of an estrogen as part of a combination therapy.

In another embodiment, the invention provides a method of treating or reducing the risk of acquiring these above-mentioned diseases comprising administering to said patient a therapeutically effective amount of an agonist/antagonist estrogen (mixed SERM) and further comprising administering to said patient a therapeutically effective amount of a pure selective estrogen receptor modulator (pure-SERM) or estrogen as part of a combination therapy. As used herein, “mixed SERM” means that the SERM has some estrogenic activities in breast and endometrium tissues at physiological or pharmacological concentrations. As used herein, “Pure SERM” means that the SERM does not have any estrogenic activity in breast and endometrial tissues at physiological or pharmacological concentrations.

In another embodiment, the invention provides a kit comprising a first container containing a therapeutically effective amount of at least one estrogen and further comprising a second container containing a therapeutically effective amount of at least one selective estrogen receptor modulator.

In another embodiment, the invention provides a pharmaceutical composition comprising: a) a pharmaceutically acceptable excipient, diluent or carrier; b) a therapeutically effective amount of at least one estrogen; and c) a therapeutically effective amount of at least one selective estrogen receptor modulator.

As used herein) compounds administered to a patient “in association with” other compounds are administered sufficiently close to administration of said other compound that a patient obtains the physiological effects of both compounds simultaneously, even though the compounds were not administered in close time proximity. When compounds are administered as part of a combination therapy they are administered in association with each other.

The estrogen replacement therapy is commonly used in postmenopausal women to prevent and treat diseases due to the menopause, namely osteoporosis, hot flashes, coronary heart disease (Cummings 1991) but presents some undesirable effects associated with chronic estrogen administration. Particularly, the perceived increased risk for uterine and/or breast cancer (Judd, Meldrum et al. 1983; Colditz, Hankinson et al. 1995) generated by estrogen is the major disadvantage of this therapy. The authors of the present invention have found that the addition of a selective estrogen receptor modulator (SERM) to estrogen administration suppresses these undesirable effects.

The invention provides a method of treating or reducing the risk of acquiring breast tenderness induced by hormone replacement therapy (HRT) since the SERM will cause atrophy of breast epithelium, instead of the stimulation caused by HRT, breast tenderness will be reduced or eliminated.

The invention also provides a method of prevention and treatment of vaginal bleeding induced by hormone replacement therapy (HRT). Since the SERM will cause endometrial atrophy, vaginal bleeding will not occur.

On the other hand, SERMs alone have little or no beneficial effects on some menopausal symptoms like hot flashes and sweats. The applicant believes that the addition of an estrogen to SERM treatment of menopausal symptoms reduces or even eliminates hot flashes and sweats. It is important to note that hot flashes and sweats are the first manifestations of menopause and the acceptation or non-acceptation of menopausal treatment by patients is usually dependent upon the success or non-success in the reduction of hot flashes and sweats.

As used herein, a selective estrogen receptor modulator (SERM) is a compound that either directly or through its active metabolite functions as an estrogen receptor antagonist (“antiestrogen”) in breast tissue, yet provides estrogenic or estrogen-like effect on bone tissue and on serum cholesterol levels (i.e. by reducing serum cholesterol). Non-steroidal compounds that function as estrogen receptor antagonists in vitro or in human or rat breast tissue (especially if the compound acts as an antiestrogen on human breast cancer cells) is likely to function as a SERM. Conversely, steroidal antiestrogens tend not to function as SERMs because they tend not to display any beneficial effect on serum cholesterol. Non-steroidal antiestrogens we have tested and found to function as SERMs include EM-800, EM-652.HCl, Raloxifene, Tamoxifen, 4-hydroxy-Tamoxifen, Toremifene, 4-hydroxy-Toremifene, Droloxifene, LY 353 381, LY 335 563, GW-5638, Lasofoxifene, TSE 424 and Idoxifene, but are not limited to these compounds. But we have found also that all SERMs do not react in the same manner and may be divided into two subclasses: “pure SERMs” and “mixed SERMs”. Thus, some SERMs like EM-800 and EM-652.HCl do not have any estrogenic activity in breast and endometrial tissues at physiological or pharmacological concentrations and have hypocholesterolemic and hypotriglyceridemic effects in the rat. These SERMS may be called “pure SERMs”. The ideal SERM is a pure SERM of the type EM-652.HCl because of its potent and pure antiestrogenic activity in the mammary gland. Others, like Raloxifene, Tamoxifen, Droloxifene, 4-hydroxy-Tamoxifen (1-(4-dimethylaminoethoxyphenyl)-1-(4-hydroxyphenyl)-2-phenyl-but-1-ene), Toremifene, 4-hydroxy-Toremifene [(Z )-(2)-2-[4-(4-chloro-1-(4-hydroxyphenyl)-2-phenyl-1-butenyl)phenoxy]-N,N-dimethyletha namine), LY 353 381, LY 335 563, GW-5638 and Idoxifene have some estrogenic activities in the breast and endometrium. This second series of SERMs may be called “mixed SERMs”. The unwanted estrogenic activities of these “mixed SERMs”, may be inhibited by addition of pure “SERMs” as shown in FIGS. 6 and 7 in in vitro tests and in FIG. 9 in an in 7 vivo test of breast cancer. Since human breast carcinoma xenografts in nude mice are the closest available model of human breast cancer, we have thus compared the effect of EM-800 and Tamoxifen alone and in combination on the growth of ZR-75-1 breast cancer xenografts in nude mice.

For all combinations taught herein, administering separate compounds for each part of the combination is contemplated except where otherwise stated. Thus, for example, administering a SERM and an estrogen refers to administering two different compounds—not to administering a single compound that is a SERM with some estrogenic characteristics.

The applicant believes that it is very important that SERMs of the invention act as pure antiestrogens in breast, uterine, and endometrial tissues because SERMs have to counteract potential side-effects of estrogens which can increase the risk of cancer in these tissues. Particularly, the applicant believes that benzopyran derivatives of the invention having the absolute configuration 2S at position 2 is more suitable than its racemic mixture. Thus, in U.S. Pat. No. 6,060,503, optically active benzopyran antiestrogens having 2S configuration are disclosed to treat estrogen-exacerbated breast and endometrial cancer and these compounds are shown to be significantly more efficient than racemic mixtures(see FIGS. 1-5 of U.S. Pat. No. 060,503). The enantiomer of 2S configuration being difficult to be industrially obtained as a pure state, the applicant believes that less than 10%, preferably less than 5% and more preferably less than 2% by weight of contamination by the 5R enantiomer is preferred.

### BRIEF DESCRIPTION OF THE DRAWINGS

FIG. 1 shows the effect of treatment with DHEA (10 mg, percutaneously, once daily) or EM-800 (75 μg, orally, once daily) alone or in combination for 9 months on serum triglyceride (A) and cholesterol (B) levels in the rat. Data are expressed as the means±SEM. **: P<0.01 experimental versus respective control.

FIG. 2 shows: A) Effect of increasing doses of DHEA (0.3 mg, 1.0 mg or 3.0 mg) administered percutaneously twice daily on average ZR-75-1 tumor size in ovariectomized (OVX) nude mice supplemented with estrone. Control OVX mice receiving the vehicle alone are used as additional controls. The initial tumor size was taken as 100%. DHEA was administered percutaneously (p.c.) in a 0.02 ml solution of 50% ethanol—50% propylene glycol on the dorsal skin. B) Effect of treatment with increasing doses of DHEA or EM-800 alone or in combination for 9.5 months on ZR-75-1 tumor weight in OVX nude mice supplemented with estrone. **, p<0.01, treated versus control OVX mice supplemented with estrone.

FIG. 3 shows the effect of increasing oral doses of the antiestrogen EM-800 (15 μg, 50 μg or 100 μg) (B) or of percutaneous administration of increasing doses of DHEA (0.3, 1.0 or 3.0 mg) combined with EM-800 (15 μg) or EM-800 alone (A) for 9.5 months on average ZR-75-1 tumor size in ovariectomized(OVX) nude mice supplemented with estrone. The initial tumor size was taken as 100%. Control OVX mice receiving the vehicle alone were used as additional controls. Estrone was administered subcutaneously at the dose of 0.5 μg once daily while DHEA was dissolved in 50% ethanol—50% propylene glycol and applied on the dorsal skin area twice daily in a volume of 0.02 ml. Comparison is also made with OVX animals receiving the vehicle alone.

FIG. 4 shows the effect of 65-day-treatment with the antiestrogen EM-800 at the doses of 0.25 and 2.5 mg per Kg body weight (orally, once daily) or medroxyprogesterone acetate (MPA, 1 mg s.c., twice daily) or the combination of EM-800 (0.25 mg/Kg body weight) and MPA on the E₁ (1.0μg, s.c., twice daily)-stimulated growth of DMBA-induced mammary carcinoma in ovariectomized rats. The change in tumor size is expressed as % of initial tumor size. The data are expressed as means±SEM.

FIG. 5 shows the effect of 37-week treatment with increasing doses (0.01, 0.03, 0.1, 0.3, and 1 mg/kg) of EM-800 or Raloxifene administered on total serum cholesterol levels in the ovariectomized rat. Comparison is made with intact rats and ovariectomized animals bearing an implant of 17β-estradiol (E₂);** p<0.01, experimental versus OVX control rats.

FIG. 6 shows the effect of increasing concentrations of EM-800, (Z)-4-OH-Tamoxifen, (Z)-4-OH-Toremifene and Raloxifene on alkaline phosphatase activity in human Ishikawa cells. Alkaline phosphatase activity was measured after a 5-day exposure to increasing concentrations of indicated compounds in the presence or absence of 1.0 nM E₂. The data are expressed as the means±SEM of four wells. When SEM overlaps with the symbol used, only the symbol is shown (Simard, Sanchez et al. 1997).

FIG. 7 shows the blockade of the stimulatory effect of (Z)-4-OH-Tamoxifen, (Z)-4-OH-Toremifene, Droloxifene and Raloxifene on alkaline phosphatase activity by the antiestrogen EM-800 in human Ishikawa carcinoma cells. Alkaline phosphatase activity was measured after a 5-day exposure to 3 or 10 nM of the indicated compounds in the presence or absence of 30 or 100 nM EM-800. The data are expressed as the means±SD of eight wells with the exception of the control groups were data are obtained from 16 wells (Simard, Sanchez et al. 1997).

FIG. 8 shows the comparison of the effects of standard HRT (estrogen) and the SERM (EM-652) on parameters of menopause. The addition of a SERM to standard HRT will counteract the potentially negative effect of estrogens.

FIG. 9 shows that the stimulatory effect of Tamoxifen on the growth of human breast cancer ZR-75-1 xenografts is completely blocked by simultaneous administration of EM-652.HCl. EM-652.HCl, by itself, in agreement with its pure antiestrogenic activity has no effect on tumor growth in the absence of Tamoxifen.

FIG. 10 shows sections of rat mammary gland.

A. Untreated animal. The lobules (L) consist of a few alveoli. Insert. High magnification showing alveoli.

B. Animal treated with EM-800 (0.5 mg/kg, b w per day) for 12 weeks. The lobules (L) are reduced in size. Insert. High magnification showing atrophied alveolar cells.

FIG. 11 shows sections of rat endometrium.

A. Untreated animal. The luminal epithelium (LE) is characterized by columnar epithelial cells while the glandular epithelium (GE) is rather cuboidal. The stroma contain several cellular elements and collagen fibers.

B. Animal treated with EM-800 (0.5 mg/kg, b w per day) during 12 weeks. The luminal epithelium is markedly reduced in height. The glandular epithelial cells have unstained cytophasm with no sign of activity. The stroma is highly cellular due to reduction in intercellular elements of the stroma.

FIG. 12 shows the effect on uterine weight of increasing concentrations of EM-652.HCl, lasofoxifene (free base; active and inactive enantiomers) and raloxifene administered orally for 9 days to ovariectomized mice simultaneously treated with estrone. *p<0.05, **p<0.01 versus E₁-treated control.

FIG. 13 shows the effect on vaginal weight of increasing concentrations of EM-652.HCl, lasofoxifene (free base; active and inactive enantiomers) and raloxifene administered orally for 9 days to ovariectomized mice simultaneously treated with estrone. **p<0.01 versus E₁-treated control.

FIG. 14 shows the effect on uterine weight of 1 μg and 10 μg of EM-652.HCl, lasofoxifene (free base; active and inactive enantiomers) and raloxifene administered orally for 9 days to ovariectomized mice. **p<0.01 versus OVX control.

FIG. 15 shows the effect on vaginal weight of 1 μg and 10 μg of EM-652.HCl, lasofoxifene (free base; active and inactive enantiomers) and raloxifene administered orally for 9 days to ovariectomized mice. **p<0.01 versus OVX control.

FIG. 16 shows the effect of 26-week treatment with E₂, EM-652.HCl, E₂+EM-652.HCl, DHEA, DHEA+EM-652.HCl and DHEA+EM-652.HCl+E₂ on lumbar spine BMD in OVX rats having established osteopenia. Intact control and OVX control animals were included as control groups.

FIG. 17 shows the effect of 26-week treatment with E₂, EM-652.HCl, E₂+EM-652.HCl, DHEA, DHEA+EM-652.HCl and DHEA+EM-652.HCl+E₂ on femoral BMD in OVX rats having established osteopenia. Intact control and OVX control animals were included as control groups.

FIG. 18 shows the effect of 26-week treatment with E₂, EM-652.HCl, E₂+EM-652.HCl, DHEA, DHEA+EM-652.HCl and DHEA+EM-652.HCl+E₂ on total body fat in OVX rats having established osteopenia. Intact control and OVX control animals were included as control groups.

FIG. 19A shows the effects of antiestrogens on ZR-75-1 tumor growth. Effect of treatment with 7 antiestrogens for 161 days, on estrone-induced growth of human ZR-75-1 breast tumors in ovariectomized nude mice. Tumor size is expressed as the percentage of initial tumor area (Day1=100%). Data is expressed as means±SEM (n=18-30 tumors/group); ## p<0,01 vs EM-652.HCl; ** p<0,01 vs OVX. Antiestrogens were administered orally once daily at the dose of 50 μg/mouse under estrone stimulation obtained with subcutaneous 0.5-cm silastic implants containing 1:25 ratio of estrone and cholesterol.

FIG. 19B shows the effects of antiestrogens on AR-75-1 tumor growth. Effet of treatment with 7 antiestrogens for 161 days, on the growth of human ZR-75-1 breast tumors in ovariectomized nude mice. Tumor size is expressed as the percentage of initial tumor area (Day 1=100%). Date is expressed as means±SEM (n=18-30 tumors/group); ## p<0,01 vs EM-652.HCl; **p<0,01 vs OVX. Antiestrogens were administered orally once daily at the dose of 100 μg/mouse in absence of estrogen stimulation.

FIG. 19C shows the effects of antiestrogens on ZR-75-1 tumor growth. Effect of treatment with 7 antiestrogens for 161 days, on the growth of human ZR-75-1 breast tumors in ovariectomized nude mice. Tumor size is expressed as the percentage of initial tumor area (Day 1=100%). Data is expressed as means±SEM (n=18-30 tumors/group); ##p<0,01 vs EM-652.HCl; **p<0,01 vs OVX. Antiestrogens were administered orally once daily at the dose of 200 μg/mouse in absence of estrogen stimulation.

FIG. 20A shows the effects of antiestrogens on categories of response. Effect of a 161-day administration of 7 antiestrogens, on the category of response of human ZR-75-1 breast tumors in ovariectomized nude mice. Complete regression identifies those tumors that were undetectable at the end of treatment; partial regression corresponds to the tumors that regressed ≧50% of their original size; stable response refers to tumors that regressed <50% or progressed ≦50%; and progression indicates that they progressed more than 50% compared with their original size. Antiestrogens were administered orally once daily at the dose of 50 μg/mouse under estrone stimulation obtained with subcutaneous 0.5-cm silastic implants containing 1:25 ratio of estrone and cholesterol.

FIG. 20B shows the effects of antiestrogen on categories of response. Effect of a 161-day administration of 7 antiestrogens, on the category of response of human ZR-75-1 breast tumors in ovariectomized nude mice. Complete regression identifies those tumors that were undetectable at the end of treatment; partial regression corresponds to the tumors that regressed ≧50% of their original size; stable response refers to tumors that regressed <50% or progressed ≦50%; and progression indicates that they progressed more than 50% compared with their original size. Antiestrogens were administered orally once daily at the dose of 200 μg/mouse in absence of estrogen stimulation.

FIG. 20C shows the effects of antiestrogen on categories of response. Effect of a 161-day adminsitration of 7 antiestrogens, on the category of response of human ZR-75-1 breast tumors in ovariectomized nude mice. Complete regression identifies those tumors that were undetectable at the end of treatment; partial regression corresponds to the tumors that regressed ≧50% of their original size; stable response refers to tumors that regressed <50% or progressed ≦50%; and progression indicates that they progressed more than 50% compared with their original size. Antiestrogens were administered orally once daily at the dose of 200 μg/mouse in absence of estrogen stimulation.

FIG. 21 shows the effect of EM-652.HCl for 2 weeks at increasing daily doses ranging from 0.01 mg/kg to 10 mg/kg on uterine weight in ovariectomized rats supplemented with daily oral 17β-estradiol (2 mg/kg). Intact animals are used as additional controls.

FIG. 22 shows the effect of EM-652.HCl for 2 weeks at increasing daily doses ranging from 0.01 mg/kg to 10 mg/kg on endometrial epithelial height in ovariectomized rats supplemented with daily oral 17β-estradiol (2 mg/kg). Intact animals are used as additional controls.

FIG. 23. Hematoxylin and eosin-stained sections of rat uteri illustrating epithelial lining cells obtained from intact control (A), OVX control (B), OVX+E₂ (2 mg/kg) (C) and OVX+E₂+EM-652.HCl (3 mg/kg) rats treated for 14 days. The stimulatory effect of estradiol on the endometrial epithelial cells was reversed by the simultaneous administration of EM-652.HCl. (Magnification: ×700). BM: basal membrane.

FIG. 24 shows the effect of EM-652.HCl for 2 weeks at increasing daily doses ranging from 0.01 mg/kg to 10 mg/kg on vaginal weight in ovariectomized rats supplemented with daily oral 17β-estradiol (2 mg/kg). Intact animals are used as additional controls.

FIG. 25 shows the effect of EM-652.HCl for 2 weeks at increasing daily doses ranging from 0.01 mg/kg to 10 mg/kg on serum cholesterol in ovariectomized rats supplemented with daily oral 17β-estradiol (2 mg/kg). Intact animals are used as additional controls.

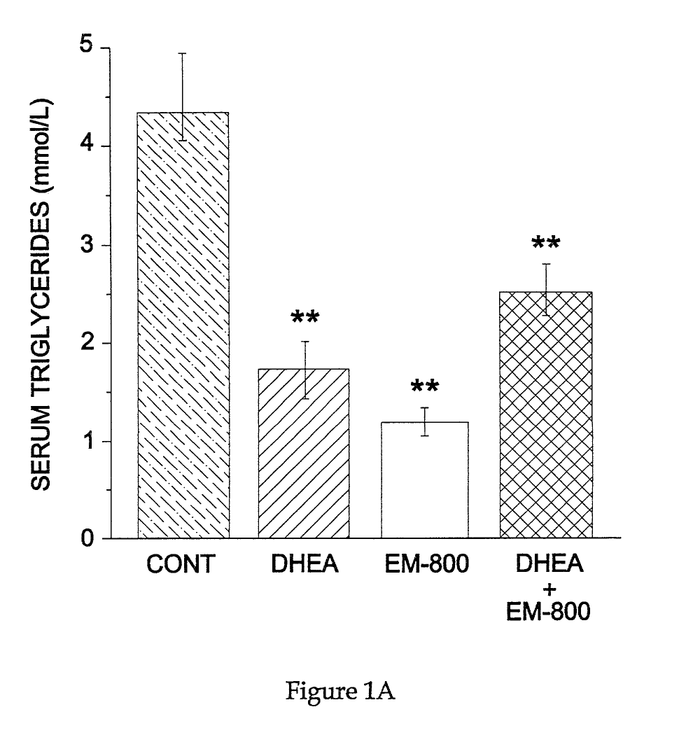

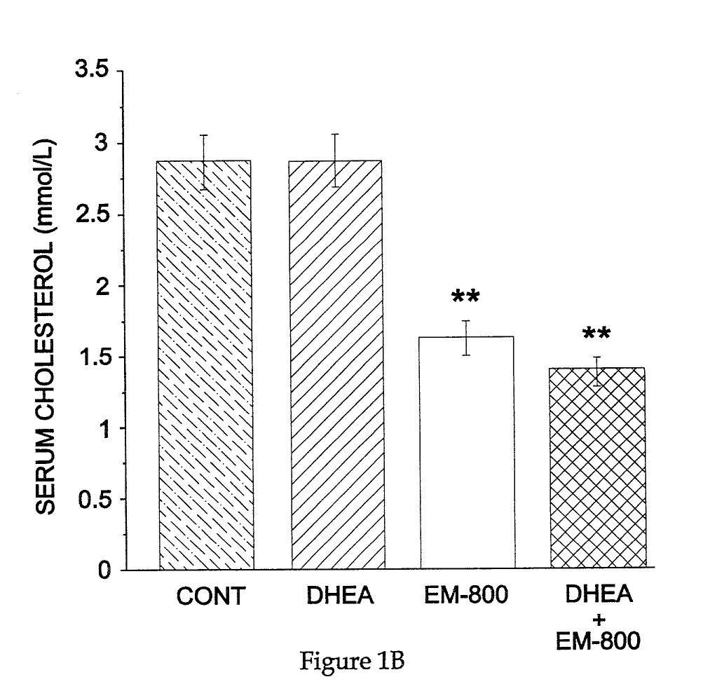

### DETAILED DESCRIPTION OF THE INVENTION

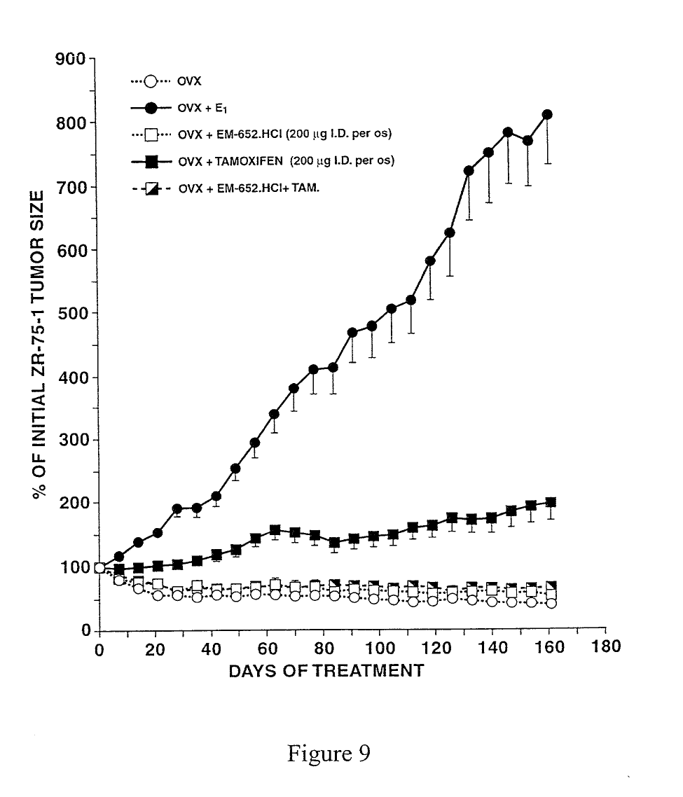

It can be seen in FIG. 9 that the approximately 100% stimulatory effect of Tamoxifen on tumor growth was completely blocked by simultaneous treatment with EM-652 HCl. EM-652.HCl in accordance with its pure antiestrogenic activity did not exert any stimulatory effect on the growth of the human breast cancer ZR-75-1 xenografts in nude mice (FIG. 9).

We have tested the steroidal antiestrogen ICI 182,780 and found it not to function as a SERMs. SERMs, in accordance with the invention, may be administered in the same dosage as known in the art, even where the art uses them as antiestrogens instead of as SERMs.

We have also noted a correlation between the beneficial effect of SERMs have on serum cholesterol and beneficial estrogenic or estrogen-like effects on bone. SERMs have also a beneficial effect on hypertension, insulin resistance, diabetes, and obesity (especially abdominal obesity). Without intending to be bound by theory, it is believed that SERMs, many of which preferably have two aromatic rings linked by one to two carbon atoms, are expected to interact with the estrogen receptor by virtue of the foregoing portion of the molecule that is best recognized by the receptor. Preferred SERMs have side chains which may selectively cause antagonistic properties in breast and usually uterine tissues without having significant antagonistic properties in other tissues. Thus, the SERMs may desirably functions as antiestrogens in the breast while surprisingly and desirably functioning as estrogens (or providing estrogen-like activity) in bone and in the blood (where concentrations of lipid and cholesterol are favorably affected). The favorable effect on cholesterol and lipids translates to a favorable effect against atherosclerosis which is known to be adversely, affected by improper levels of cholesterol and lipids.

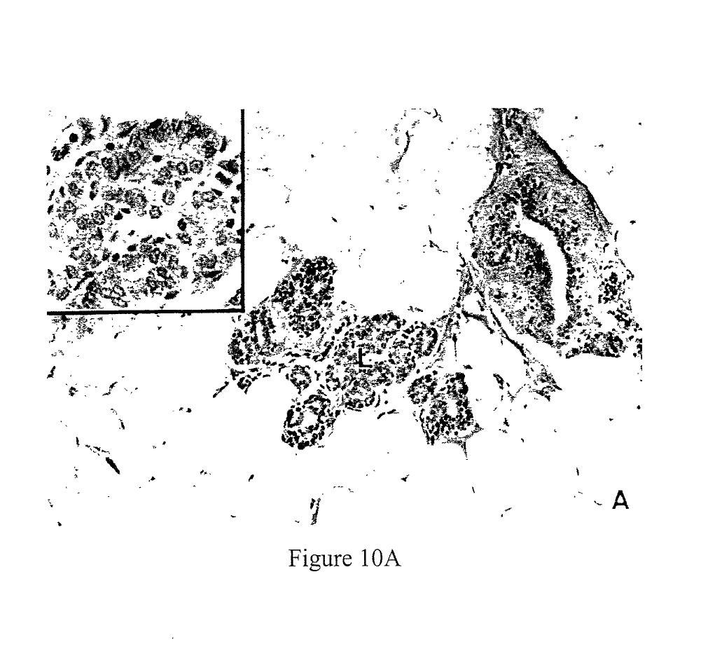

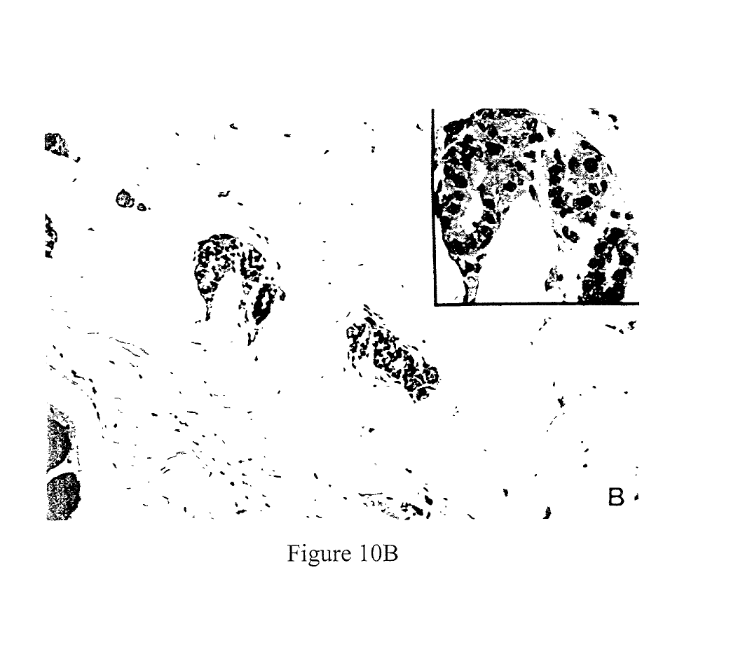

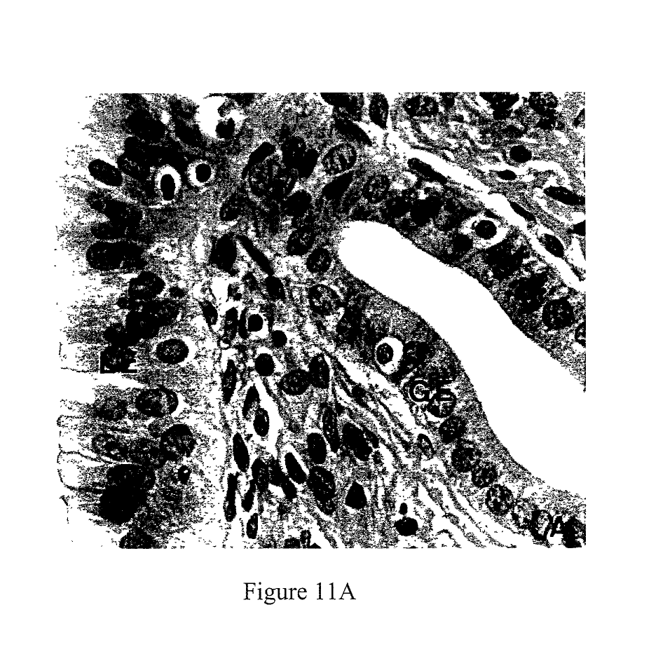

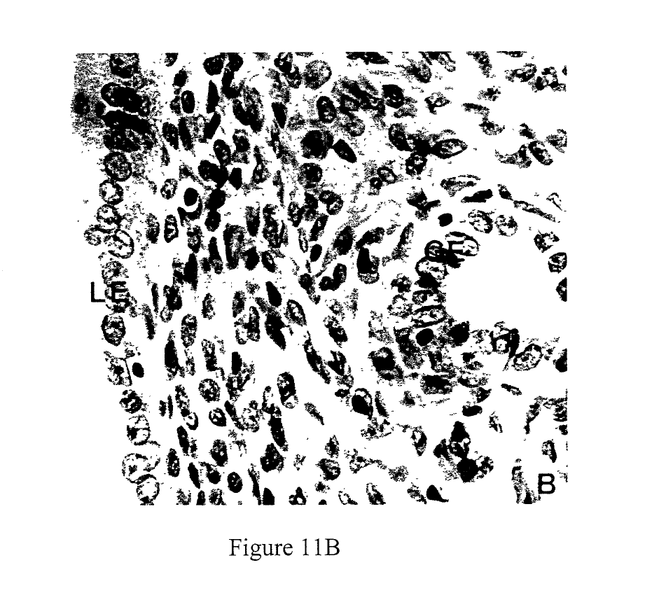

On the other hand, osteoporosis, hypercholesterolemia, hyperlipidemia, cognition and atherosclerosis respond favorably to estrogenic or estrogen-like activity. By using estrogens in combination with SERMs in accordance with the invention, desirable effects are provided in target tissues without undesirable effects in certain other tissues. For example, the combination of an estrogen and a SERM can have favorable estrogenic effect in the bone (or on lipid or cholesterol) while avoiding unfavorable estrogenic effect in the breast and uterus since the SERMs will, acting as estrogen antagonists, efficiently block the effect of estrogen in the breast and endometrium as seen in FIGS. 10 and 11.

As demonstrated in FIG. 10, although circulating levels of 17β-estradiol were elevated from 95.9±32.4 pg/ml in intact animals to 143.5±7.8 pg/ml (50% elevation in animals treated with EM-800, 0.5 mg/kg, orally daily/for 12 weeks), a marked atrophy of the mammary gland was observed. Similarly, in FIG. 11, a marked atrophy of the endometrium was observed in animals receiving EM-800 (0.5 mg/kg). In these intact animals receving the pure antiestrogen EM-800, the inhibitory effect of estrogens at the hypothalamo-pituitary level was removed, thus causing increased LH and then secondarily increased 17β-estradiol secretion by the ovaries.

In a 6-month study performed in intact rats who received EM-652 at the same daily 0.5 mg/kg dose, the concentration of the antiestrogen EM-652 was measured at 0.4 ng/ml in the circulation (URMA-05-011-94). Since the average serum concentration of EM-652 in women who received a daily oral dose of EM-800 of 20 mg was measured at 7.3±0.77 ng/ml, it is clear that the administration of estrogen replacement therapy in postmenopausal women will not affect the potent inhibitory effect of EM-652 and its ability to prevent breast and endometrial cancer. Phase I studies have shown that EM-800 and EM-652.HCl give almost superimposable serum levels of EM-652.

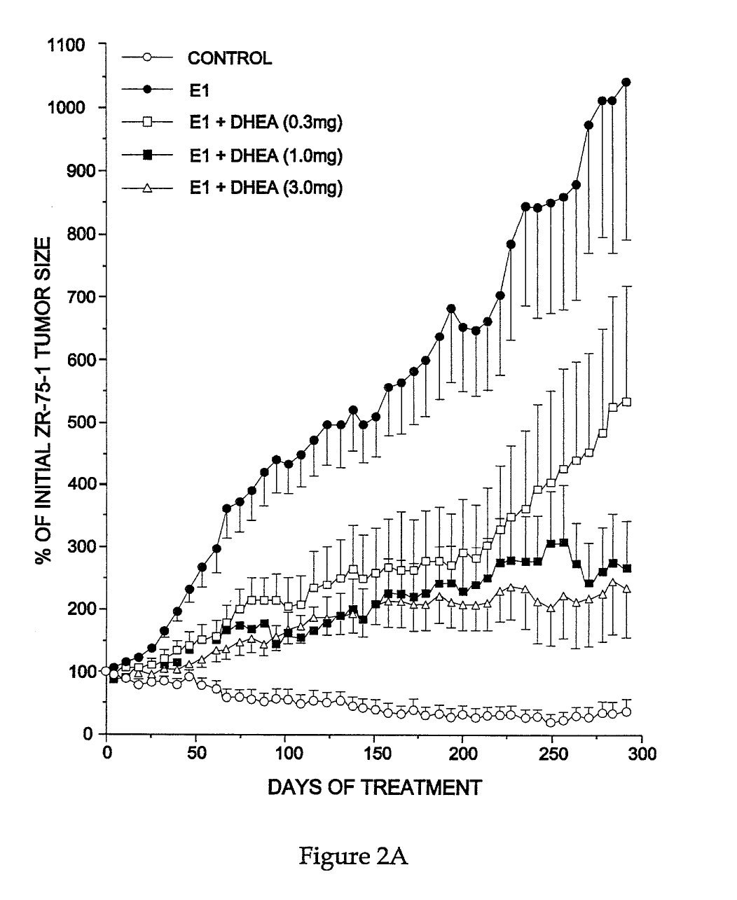

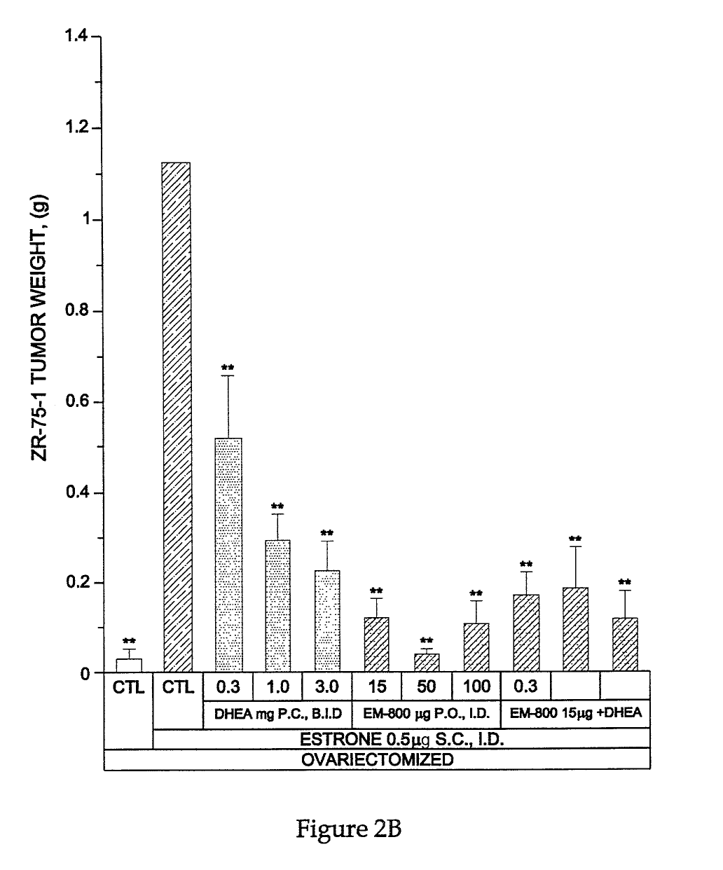

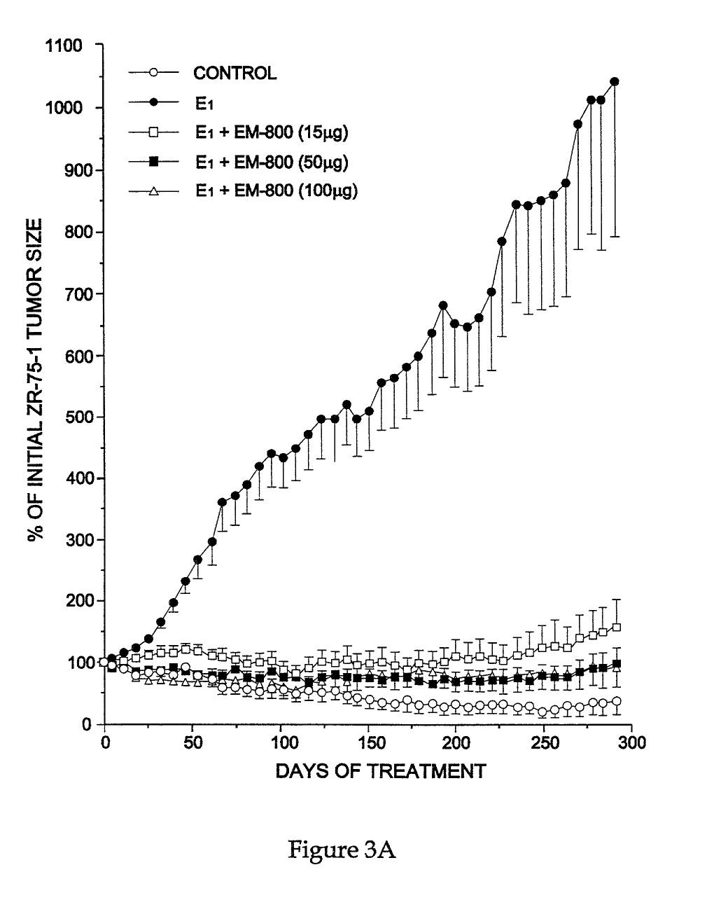

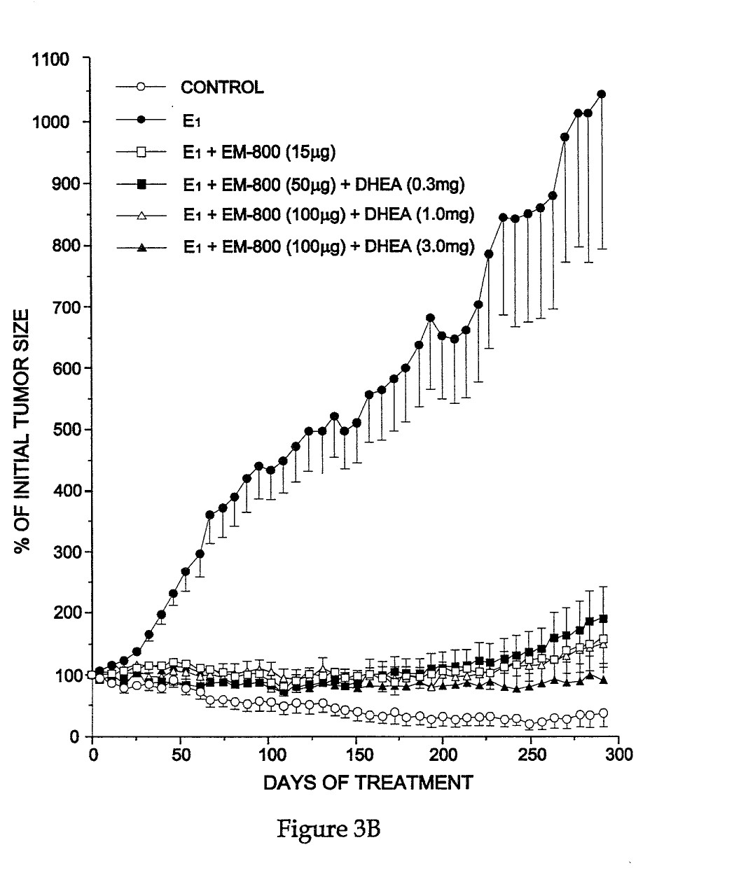

Undesirable effects are also mitigated in a synergistic way by the combination used in the invention. For all diseases discussed herein, any other effect on breast tissues that might otherwise result from estrogens given at a replacement dose is efficiently blocked by the antiestrogenic effect of the SERM in breast tissue as seen in FIG. 2 and 3. The same conclusion can be reached from FIG. 10.

In preferred embodiments, precursors of sex steroids (dehydroepiandrosterone, dehydroepiandrosterone-sulfate, androst-5-ene-3β,17β-diol, 4-androstene-3,17-dione and a prodrug of thereof) or androgenic agents are added to provide beneficial androgenic effects, particularly in the reduction of the risk of acquiring, or in the treatment of bone diseases. The combination of a SERM and an estrogen in the treatment of osteoporosis reduce or even stop the degradation of the bone. While the further addition of an androgen or DHEA (and other precursors of sex steroids) permit the rebuilding of the damaged bone tissues. Precursors of the sex steroids which have other beneficial effects in the treatment of hypercholesterolemia, hyperlipidemia, menopausal syndrome, Alzheimer's disease, cardiovascular diseases, breast cancer, uterine cancer, and ovarian cancer, can act in synergy with the combination of SERM and estrogen for a better treatment of above-mentioned diseases. This synergistic effect is due to the fact than androgens (or precursors of sex steroids metabolised into androgens in the peripheral tissues) and estrogens or SERMs act by different mechanisms.

In some embodiments, progestins are added to provide further androgenic effect. Progestins may be used at low dosages known in the art without adversely affecting receptors other than the androgen receptors (e.g. glucocorticoid receptors). They also are relatively free of unwanted androgenic side effects (such as facial hair with female patients).

Hot flashes, cardiovascular symptoms, Alzheimer's disease, loss of cognitive functions and insomnia involve certainly estrogen receptors situated in the nervous central system. Probably, low levels of estrogens in the brain, can explain at least in part, these conditions. Exogenous estrogens and particularly estradiol can pass through the brain barrier and bind to the estrogen receptor to restore the normal estrogenic action. On the other hand, SERMs of the invention, and more particularly those of EM-652.HCl family, cannot pass through the brain barrier as shown in example 9. Thus, they cannot antagonise the positive effect of estrogens in brain but they antagonise the negative effects of estrogens in the breast, uterine, and endometrial tissues rending this combination (SERM+estrogens) particularly attractive for the treatment or reduction of the risk of acquiring the above-mentioned conditions.

Overall Additive Benefits of Combining an Estrogen and a SERM

The main reason why women consult their physician at menopause is the occurrence of hot flashes, a problem well known to be eliminated by estrogen replacement therapy. Since the site responsible for hot flashes is the central nervous system (CNS) and EM-652 has very poor accessibility to the CNS (data enclosed), it is expected that estrogen administration will control hot flashes without interference by the SERM. On the other hand, the SERM will eliminate all the negative effects of estrogens at other sites, specially the risk of breast and uterine cancer. In fact, the addition of EM-652 to estrogens blocks the stimulatory effect of estrogens on the mammary gland and uterus while, in other tissues, EM-652 will exert its own beneficial effect, for example on the bone, where it partially reverses the effect of ovariectomy on bone mineral density.

No adverse effect of EM-652 is seen on any parameter while it should exert marked beneficial effects for the prevention and treatment of breast and uterine cancer.

The present data show that the addition of EM-652 blocks the stimulatory effect of estrogen on the mammary gland and uterus (examples 4, 8 and 10) while, in other tissues, EM-652.HCl exerts its own beneficial effects. For example, in the bone (example 5), EM-652 partially reverses the effect of ovariectomy on bone mineral density. Such an effect has led to the commercialisation of raloxifene for the treatment of osteoporosis in post-menopausal women. In fact, raloxifene has been found to be 3 to 10 times less potent than EM-652 to prevent BMD loss in the rat (Martel et al., J Steroid Biochem Molec Biol 2000:74, pp 45-56). Although the effect of SERMs on BMD, as shown for other SERMs like raloxifene, is not as complete as achieved with estrogens, the effect on bone fractures observed in post-menopausal women has been found to be the same with estrogens and the SERM raloxifene. It is thus expected that although BMD is not reversed completely by EM-652 or other SERMs, the effect on bone fractures, which is the most important parameter of response, is as important as the one seen after the use of estrogens. Moreover, it is quite possible, as suggested, that BMD measurements do not provide the complete picture of the effect of a compound on bone physiology.

The important aspect is that while treatment with a SERM exerts beneficial effects on bone, its combination with an estrogen, given mainly to block hot flashes, permits to decrease the risk of breast and uterine cancers associated with the use of estrogen alone.

Preferred SERMs discussed herein relate: (1) to all diseases stated to be susceptible to the invention; (2) to both therapeutic and prophylactic applications; and (3) to preferred pharmaceutical compositions and kits.

A patient in need of treatment or of reducing the risk of onset of a given disease is one who has either been diagnosed with such disease or one who is susceptible of acquiring such disease.

Except where otherwise stated, the preferred dosage of the active compounds (concentrations and modes of administration) of the invention is identical for both therapeutic and prophylactic purposes. The dosage for each active component discussed herein is the same regardless of the disease being treated (or of the disease whose likelihood of onset is being reduced).

Except when otherwise noted or where apparent from context, dosages herein refer to weight of active compounds unaffected by pharmaceutical excipients, diluents, carriers or other ingredients, although such additional ingredients are desirably included, as shown in the examples herein. Any dosage form (capsule, tablet, injection or the like) commonly used in the pharmaceutical industry is appropriate for use herein, and the terms “excipient”, “diluent”, or “carrier” include such nonactive ingredients as are typically included, together with active ingredients in such dosage forms in the industry. For example, typical capsules, pills, enteric coatings, solid or liquid diluents or excipients, flavorants, preservatives, or the like may be included.

All of the active ingredients used in any of the therapies discussed herein may be formulated in pharmaceutical compositions which also include one or more of the other active ingredients. Alternatively, they may each be administered separately but sufficiently simultaneous in time so that a patient eventually has elevated blood levels or otherwise enjoys the benefits of each of the active ingredients (or strategies) simultaneously. In some preferred embodiments of the invention, for example, one or more active ingredients are to be formulated in a single pharmaceutical composition. In other embodiments of the invention, a kit is provided which includes at least two separate containers wherein the contents of at least one container differs, in whole or in part, from the contents of at least one other container with respect to active ingredients contained therein.

Combination therapies discussed herein also include use of one active ingredient (of the combination) in the manufacture of a medicament for the treatment (or risk reduction) of the disease in question where the treatment or prevention further includes another active ingredient of the combination in accordance with the invention. For example in one embodiment, the invention provides the use of a SERM in the preparation of a medicament for use, in combination with an estrogen and pro-drugs converted to estrogen, in vivo, in the treatment of any of the diseases for which the present combination therapy is believed effective (i.e., osteoporosis, cardiovascular diseases, hypercholesterolemia, hyperlipidemia, atherosclerosis, hypertension, insulin resistance, diabetes, obesity, hot flashes, sweat, irregular menstruation, Alzheimer's disease, cognition problems, any symptoms related to menopause, and vaginal dryness). In another embodiment, the invention provides the use of an estrogen selected from the group consisting of 17β-estradiol, 17β-estradiol esters (i.e. benzoate, cypionate, dienanthate, valerate, etc.), 17α-estradiol, 17α-estradiol esters, estriol, estriol esters, estrone, estrone esters, conjugated estrogen, equilin, equilin esters, 17α-ethynylestradiol, 17α-ethynylestradiol esters, dienestrol, mestranol, mestranol esters, DES, phytoestrogen (s), tibolone, ethynediol in the preparation of a medicament for use, in combination with a SERM, for treatment of any of those same diseases.

Estrogens are well-known to stimulate the proliferation of breast epithelial cells and cell proliferation itself is thought to increase the risk of cancer by accumulating random genetic errors that may result in neoplasia (Preston Martin et al., Cancer. Res. 50: 7415-21, 1990). Based on this concept, antiestrogens have been introduced to prevent breast cancer with the objective of reducing the rate of cell division stimulated by estrogens.

The loss of ovarian cyclicity found in female Sprague-Dawley rats after 10 months of age is accompanied by increased serum estrogen and prolactin levels and decreased serum androgen and progesterone concentrations (Lu et al., 61st Annual Meeting of the Endocrine Society 106 (abst. #134), 1979; Tang et al., Biol. Reprod. 31: 399-413, 1984; Russo et al., Monographs on Pathology of Laboratory Animals: Integument and Mammary Glands 252-266, 1989; Sortino and Wise, Endocrinology 124: 90-96, 1989; Cardy, Vet. Pathol. 28: 139-145, 1991). These hormonal changes that spontaneously occur in aging female rats are associated with multifocal proliferation and increased secretory activity of the acinar/alveolar tissue as well as mammary gland duct dilatation and formation of cysts (Boorman et al., 433, 1990; Cardy, Vet. Pathol. 28: 139-145, 1991). It should be mentioned that hyperplastic and neoplastic changes of the rat mammary gland are often accompanied by increased levels of estrogens and prolactin (Meites, J. Neural. Transm. 48: 25-42, 1980). Treatment with EM-800, a SERM of the present invention, induces atrophy of the mammary gland which is characterized by a decrease in the size and number of the lobular structures, and no evidence of secretory activity, indicating the potent antiestrogenic activity of EM-800 in the mammary gland (Luo et al. Endocrinology 138: 4435-4444, 1997).

Estrogens are known to lower serum cholesterol but to increase or to have no effect on serum triglycerides levels (Love et al., Ann. Intern. Med. 115: 860-864, 1991; Walsh et al., New Engl. J. Med. 325: 1196-1204, 1991; Barrett-Connor, Am. J. Med. 95 (Suppl. 5A): 40S-43S, 1993; Russell et al., Atherosclerosis 100: 113-122, 1993; Black et al., J. Clin. Invest. 93: 63-69, 1994; Dipippo et al., Endocrinology 136: 1020-1033, 1995; Ke et al., Endocrinology 136: 2435-2441, 1995). FIG. 3 shows that EM-800 possesses both hypocholesterolemic and hypotriglyceridemic effects in the rat, thus showing its unique action on the serum lipid profile which is apparently different from other SERMs, such as tamoxifen (Bruning et al., Br. J. Cancer 58: 497-499, 1988; Love et al., J. Natl. Cancer Inst. 82: 1327-1332, 1990; Dipippo et al., Endocrinology 136: 1020-1033, 1995; Ke et al., Endocrinology 136: 2435-2441, 1995), droloxifene (Ke et al., Endocrinology 136: 2435-2441, 1995), and raloxifene (Black et al., J. Clin. Invest. 93: 63-69, 1994). Thus, it is believed that a combination of estrogen and EM-800 should preserved the hypocholesterolemic and hypotriglyceridemic effects of EM-800, thus suggesting that such a combination could exert beneficial effects on serum lipids.

It should be mentioned that the serum lipid profile is markedly different between rats and humans. However, since an estrogen receptor-mediated mechanism is involved in the hypocholesterolemic effect of estrogens as well as antiestrogens (Lundeen et al., Endocrinology 138: 1552-1558, 1997), the rat remains a useful model to study the cholesterol-lowering effect of estrogens and “antiestrogens” in humans.

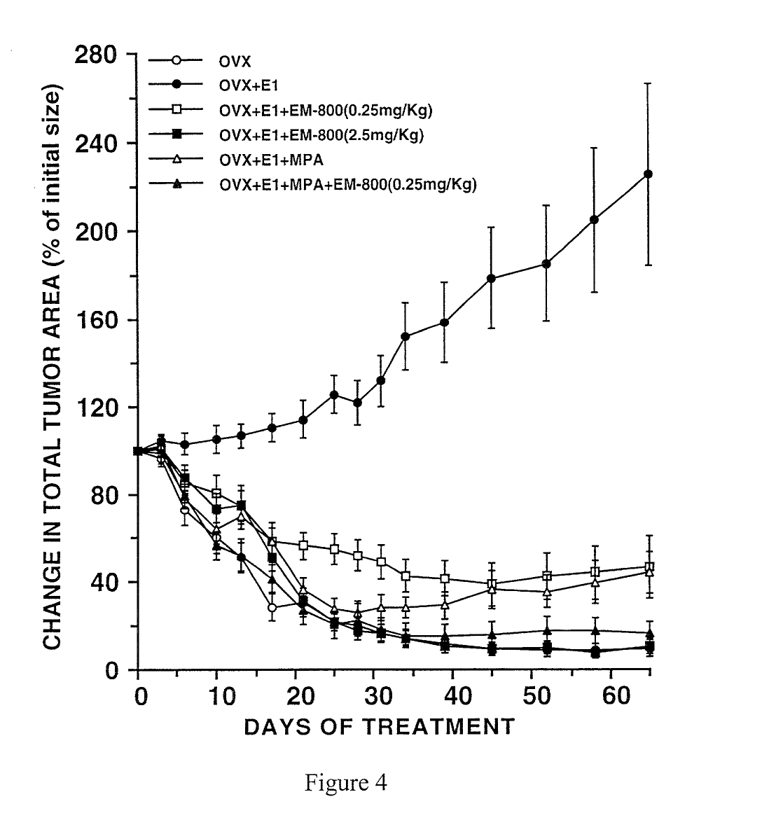

We have also studied the potential interaction of the inhibitory effect of the novel antiestrogen (EM-800) with that of sex steroid precursor (DHEA) on the growth of human ZR-75-1 breast cancer xenografts in nude mice by combined administration of the two drugs. FIGS. 2 and 3 show that DHEA, by itself, at the doses used, causes a 50 to 80% inhibition of tumor growth while the near complete inhibition of tumor growth achieved with a low dose of the antiestrogen was not affected by DHEA. A similar effect is observed with EM-800 and the progestogen, MPA on E₂-stimulated growth of DMBA-induced mammary carcinoma in ovariectomized rats as shown in FIG. 4.

The limitations of bone mineral density (BMD) measurements are well known. As an example, BMD measurements showed no change in rats treated with the steroidal antiestrogen ICI 182780 (Wakeling, Breast Cancer Res. Treat. 25: 1-9, 1993) while inhibitory changes were seen by histomorphometry (Gallagher et al., Endocrinology 133: 2787-2791, 1993). Similar differences were reported with Tamoxifen (Jordan et al., Breast Cancer Res. Treat. 10: 31-35, 1987; Sibonga et al., Breast Cancer Res. Treatm. 41: 71-79, 1996).

It should be indicated that reduced bone mineral density is not the only abnormality associated with reduced bone strength. (Guidelines for preclinical and clinical evaluation of agents used in the prevention or treatment of postmenopausal osteoporosis, Division of Metabolism and Endocrine Drug Products, FDA, May 1994). It is thus important to analyze the changes in biochemical parameters of bone metabolism induced by various compounds and treatments in order to gain a better knowledge of their action.

It is particularly important to indicate that the combination of DHEA and EM-800 exerted unexpected beneficial effects on important biochemical parameters of bone metabolism. In fact, DHEA alone did not affect the urinary hydroxyproline/creatinine ratio, a marker of bone resorption. Moreover, no effect of DHEA could be detected on daily urinary calcium or phosphorus excretion (Luo et al., Endocrinology 138: 4435-4444, 1997). EM-800, decreased the urinary hydroxyproline/creatinine ratio by 48% while, similarly to DHEA, no effect of EM-800 was seen on urinary calcium or phosphorus excretion. EM-800, moreover, had no effect on serum alkaline phosphatase activity, a marker of bone formation while DHEA increased the value of the parameter by about 75% (Luo et al., Endocrinology 138: 4435-4444, 1997).

One of the unexpected effects of the combination of DHEA and EM-800 relates to the urinary hydroxyproline/creatinine ratio, a marker of bone resorption, which was reduced by 69% when both DHEA and EM-800 were combined, this value being statistically different (p<0.01) from the 48% inhibition achieved by EM-800 alone while DHEA alone did not show any effect. Thus, the addition of DHEA to EM-800 increases by 50% the inhibitory effect of EM-800 on bone reabsorption. Most importantly, another unexpected effect of the addition of DHEA to EM-800 was the approximately 84% decrease in urinary calcium ( from 23.17±1.55 to 3.71±0.75 μmol/24 h/100 g (p<0.01) and the 55% decrease in urinary phosphorus (from 132.72±6.08 to 59.06±4.76 timol/24 h/100 g (p<0.01) respectively, (Luo et al., Endocrinology 138: 4435-4444, 1997).

TABLE 1

| | URINE | | | SERUM |
|---|---|---|---|---|
| | CALCIUM | PHOSPHORUS | HP/Cr | tALP |
| GROUP | (μmol/24 h/100 g) | (μmol/24 h/100 g) | (μmol/mmol) | (IU/L) |
| CONTROL | 23.17 ± 1.55 | 132.72 ± 6.08 | 13.04 ± 2.19 | 114.25 ± 14.04 |
| DHEA (10 mg) | 25.87 ± 3.54 | 151.41 ± 14.57 | 14.02 ± 1.59 | 198.38 ± 30.76* |
| EM-800 (75 μg) | 17.44 ± 4.5 | 102.03 ± 25.13 | 6.81 ± 0.84** | 114.11 ± 11.26 |
| DHEA + EM-800 | 3.71 ± 0.75** | 59.06 ± 4.76** | 4.06 ± 0.28** | 204.38 ± 14.20** |

It is also of interest to note that the potent inhibitory effect of EM-800 on serum cholesterol is not prevented by simultaneous treatment with DHEA (Luo et al., Endocrinology 138: 4435-4444, 1997).

While Raloxifene and similar compounds prevent bone loss and decrease serum cholesterol (like estrogens), it should be mentioned that when Raloxifene was compared to Premarin on BMD, the effect of Raloxifene on BMD was less potent than that of Premarin (Minutes of the Endocrinology and Metabolism Drugs Advisory Committee, FDA Thursday, Meeting #68, Nov. 20, 1997).

The bone loss observed at menopause in women is believed to be related to an increase in the rate of bone resorption which is not fully compensated by the secondary increase in bone formation. In fact, the parameters of both bone formation and bone resorption are increased in osteoporosis and both bone resorption and formation are inhibited by estrogen replacement therapy. The inhibitory effect of estrogen replacement on bone formation is thus believed to result from a coupled mechanism between bone resorption and bone formation, such that the primary estrogen-induced reduction in bone resorption entrains a reduction in bone formation (Parfitt, Calcified Tissue International 36 Suppl. 1: S37-S45, 1984).

Cancellous bone strength and subsequent resistance to fracture do not only depend upon the total amount of cancellous bone but also on the trabecular microstructure, as determined by the number, size, and distribution of the trabeculae. The loss of ovarian function in postmenopausal women is accompanied by a significant decrease in total trabecular bone volume (Melsen et al., Acta Pathologica & Microbiologica Scandinavia 86: 70-81, 1978; Vakamatsou et al., Calcified Tissue International 37: 594-597, 1985), mainly related to a decrease in the number and, to a lesser degree, in the width of trabeculae (Weinstein and Hutson, Bone 8: 137-142, 1987).

In order to facilitate the combination therapy aspect of the invention, for any indication discussed herein, the invention contemplates pharmaceutical compositions which include the SERM and the estrogen in a single composition for simultaneous administration. The composition may be suitable for administration in any traditional manner including but not limited to oral administration, subcutaneous injection, intramuscular injection or percutaneous administration. In other embodiments, a kit is provided wherein the kit includes one or more SERM and estrogen in separate or in one container. The above-described pharmaceutical compositions and kits may further contain a bisphosphonate compound when used for the treatment or prevention of osteoporosis. The kit may include appropriate materials for oral administration, e.g. tablets, capsules, syrups and the like and for transdermal administration, e.g., ointments, lotions, gels, creams, sustained release patches and the like.

Applicants believe that administration of estrogens, SERMs and sex steroid precursors has utility in development of osteoporosis, hypercholesterolemia, hyperlipidemia, atherosclerosis, hypertension, insulin resistance, diabetes, obesity, Alzheimer's disease and in the treatment and/or reduction of the incidence of hot flashes and sweat. The active ingredients of the invention (whether estrogen, SERM or precursor or otherwise) may be formulated and administered in a variety of ways. When administered together in accordance with the invention, the active ingredients may be administered simultaneously or separately.

Active ingredient for transdermal or transmucosal is preferably present at from 0.01% to 20% by weight relative to the total weight of the pharmaceutical composition more preferably between 2 and 10%. 17β-estradiol, estrone, conjugated estrogens should be from 0.01% to 1%, DHEA or 5-diol should be at a concentration of at least 7% for percutaneous administration. Alternatively, the active ingredient may be placed into a transdermal patch having structures known in the art, for example, structures such as those set forth in E.P. Patent No.0279982.

When formulated as an ointment, lotion, gel or cream or the like, the active compound is admixed with a suitable carrier which is compatible with human skin or mucosa and which enhances transdermal penetration of the compound through the skin or mucosa. Suitable carriers are known in the art and include but are not limited to Klucel HF and Glaxal base. Some are commercially available, e.g., Glaxal base available from Glaxal Canada Limited Company. Other suitable vehicles can be found in Koller and Buri, S. T. P. Pharma 3(2), 115-124, 1987. The carrier is preferably one in which the active ingredient(s) is (are) soluble at ambient temperature at the concentration of active ingredient that is used. The carrier should have sufficient viscosity to maintain the inhibitor on a localized area of skin or mucosa to which the composition has been applied, without running or evaporating for a time period sufficient to permit substantial penetration of the precursor through the localized area of skin or mucosa and into the bloodstream where it will cause a desirable clinical effect. The carrier is typically a mixture of several components, e.g. pharmaceutically acceptable solvents and a thickening agent. A mixture of organic and inorganic solvents can aid hydrophylic and lipophylic solubility, e.g. water and an alcohol such as ethanol.

Preferred sex steroid precursors are dehydroepiandrosterone (DHEA) (available from Diosynth Inc., Chicago, Ill., USA).

The carrier may also include various additives commonly used in ointments and lotions and well known in the cosmetic and medical arts. For example, fragrances, antioxidants, perfumes, gelling agents, thickening agents such as carboxymethylcellulose, surfactants, stabilizers, emollients, coloring agents and other similar agents may be present. When used to treat systemic diseases, the site of application on the skin should be changed in order to avoid excess local concentration of active ingredient and possible overstimulation of the skin be the active ingredient.

Treatment in accordance with the invention is suitable for indefinite continuation. The estrogen compound, SERM compound and/or the sex steroid precursor and/or bisphosphonate can also be administered, by the oral route, and may be formulated with conventional pharmaceutical excipients, e.g. spray dried lactose, microcrystalline cellulose, and magnesium stearate into tablets or capsules for oral administration.

The active substance can be worked into tablets or dragee cores by being mixed with solid, pulverulent carrier substances, such as sodium citrate, calcium carbonate or dicalcium phosphate, and binders such as polyvinyl pyrrolidone, gelatin or cellulose derivatives, possibly by adding also lubricants such as magnesium stearate, sodium lauryl sulfate, “Carbowax” or polyethylene glycol. Of course, taste-improving substances can be added in the case of oral administration forms.

As further forms, one can use plug capsules, e.g. of hard gelatin, as well as closed solf-gelatin capsules comprising a softner or plasticizer, e.g. glycerine. The plug capsules contain the active substance preferably in the form of granulate, e.g. in mixture with fillers, such as lactose, saccharose, mannitol, starches, such as potato starch or amylopectin, cellulose derivatives or highly dispersed silicic acids. In solf-gelatin capsules, the active substance is preferably dissolved or suspended in suitable liquids, such as vegetable oils or liquid polyethylene glycols.

The lotion, ointment, gel or cream should be thoroughly rubbed into the skin so that no excess is plainly visible, and the skin should not be washed in that region until most of the transdermal penetration has occurred preferably at least 4 hours and, more preferably, at least 6 hours.

A transdermal patch may be used to deliver precursor in accordance with known techniques. It is typically applied for a much longer period, e.g., 1 to 4 days, but typically contacts active ingredient to a smaller surface area, allowing a slow and constant delivery of active ingredient.

A number of transdermal drug delivery systems that have been developed, and are in use, are suitable for delivering the active ingredient of the present invention. The rate of release is typically controlled by a matrix diffusion, or by passage of the active ingredient through a controlling membrane.

Mechanical aspects of transdermal devices are well known in the rat, and are explained, for example, in U.S. Pat. Nos. 5,162,037, 5,154,922, 5,135,480, 4,666,441, 4,624,665, 3,742,951, 3,797,444, 4,568,343, 5,064,654, 5,071,644, 5,071,657, the disclosures of which are incorporated herein by reference. Additional background is provided by European Patent 0279982 and British Patent Application 2185187.

The device may be any of the general types known in the art including adhesive matrix and reservoir-type transdermal delivery devices. The device may include drug-containing matrixes incorporating fibers which absorb the active ingredient and/or carrier. In a reservoir-type device, the reservoir may be defined by a polymer membrane impermeable to the carrier and to the active ingredient.

In a transdermal device, the device itself maintains active ingredient in contact with the desired localized skin surface. In such a device, the viscosity of the carrier for active ingredient is of less concern than with a cream or gel. A solvent system for a transdermal device may include, for example, oleic acid, linear alcohol lactate and dipropylene glycol, or other solvent systems known in the art. The active ingredient may be dissolved or suspended in the carrier.

For attachment to the skin, a transdermal patch may be mounted on a surgical adhesive tape having a hole punched in the middle. The adhesive is preferably covered by a release liner to protect it prior to use. Typical material suitable for release includes polyethylene and polyethylene-coated paper, and preferably silicone-coated for ease of removal. For applying the device, the release liner is simply peeled away and the adhesive attached to the patient's skin. In U.S. Pat. No. 5,135,480, the disclosure of which is incorporated by reference, Bannon et al. describe an alternative device having a non-adhesive means for securing the device to the skin.

It is necessary only that SERM, estrogen and eventually sex steroid precursor be administered in a manner and at a dosage sufficient to allow blood serum concentration of each to obtain desired levels. In accordance with the combination therapy of the invention, concentration of the SERM is maintained within desired parameters at the same time that estrogen concentration is maintained within desired parameters.

Where estradiol is used, serum estradiol concentration should typically be maintained between 50 and 300 nanograms per liter, preferably between 100 and 200 nanograms per liter and most preferably between 150 and 175 nanograms per liter. Where another estrogen is used, serum concentration may be varied in a known manner to account for the difference in estrogenic activity relative to estradiol and in order to achieve normal per-menopausal estrogen levels. A lesser concentration is needed, for example, if Mestranol is used. Adequate serum estrogen levels can also be assessed by disappearance of the symptoms of menopause. Serum concentration of the second compound of the combination therapy (e.g., EM-652.HCl) is typically maintained between 1 and 15 micrograms per liter, or in some embodiments between 2 and 10 micrograms per liter, or between 5 and 10 micrograms per liter.

The estrogen is preferably estradiol, but may be sodium estrone sulfate or any other compound which acts as an estrogen receptor agonist. When administered separately, commercially available estrogen supplements may be used, e.g., “PREMARIN” available from Ayerst (St-Laurent, Québec, Canada). One preferred sex steroid precursor is DHEA, although DHEA-S and analogs discussed below are also especially effective for the reasons stated below. For typical patients, the appropriate dosage of estrogen to achieve desired serum concentrations is between 0.3 and 2.5 milligrams of PREMARIN per day per 50 kg of body weight when administered orally. In certain embodiments of the invention, the estrogen may be 17β-estradiol administered percutaneously in a patch which is available from CIBA under the name “ESTRADERM” wherein the daily dose is between 0.05 and 0.2 milligrams per day per 50 kg of body weight. 17β-estradiol valerianate available from Squibb under the Trade name “DELESTOGEN” is administered by injection.

Other preferred of estrogenic drug products of the invention are: patches containing 17β-estradiol available from Berlex Canada under the Trade name CLIMARA or from Novartis Pharma under the Trade name VIVELLE; vaginal device containing 17β-estradiol available Pharmacia & Upjohn under the Trade name ESTRING; gel containing 17β-estradiol available from Schering under the Trade name ESTROGEL; cream containing dienoestrol available from JANSSEN-ORTHO under the Trade name ORTHO DINESTROL.

In some embodiments the preferred estrogen is orally administered. For example micronized 17β-estradiol available from Roberts under the Trade name ESTRACE; ethinylestradiol available from Schering Canada under the Trade name ESTINYL; Estrone sulfate (estropipate) available from PHARMACIA UPJOHN under the Trade name OGEN.

In some embodiments, a mixed estrogenic/androgenic compound is preferred instead of estrogen. One of said compound is Tibolone [(7α, (7α)-17-hydroxy-7-methyl-19-norpregn-5 (10)-en-20-yn-3-one; patent number U.S. Pat. No. 3,340,279 (1967); U.S. Pat. No. 3,475,465 (1969) and Endocrinological profile described in J. de Visser et al., Arzneimittel-Forsch, 34, 1010, 1984], available from ORGANON (The Netherlands) under the trade name LIVIAL.

Drug products containing a mixture of estrogen and progestin or androgen are also preferred. Said drugs are available from Novartis Pharma under the Trade name of ESTRACOM, from Sabex under the Trade name of CLIMACTERON. The percutaneous or transmucosal delivery system of the invention may also be used as a novel and improved delivery system for the prevention and/or treatment of osteoporosis or other diseases.

Any estrogen used as required for efficacy as recommended by the manufacturer, can be used. Appropriate dosages are known in the art. Any compound or mixture of compounds having estrogenic activity or like or agonistic activity on estrogen receptors or like may be used according to the invention. (phytoestrogens, synthetic estrogens, etc.).

A selective estrogen receptor modulator of the invention has a molecular formula with the following features: a) two aromatic rings spaced by 1 to 2 intervening carbon atoms, both aromatic rings being either unsubstituted or substituted by a hydroxyl group or a group converted in vivo to hydroxyl; and b) a side chain possessing an aromatic ring and a tertiary amine function or salt thereof. One preferred SERM of the invention is EM-800 reported in PCT/CA96/00097 (WO 96/26201) The molecular structure of EM-800 is:

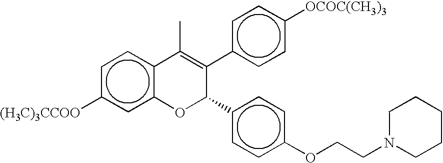

Another preferred SERM of the invention is EM-01538:

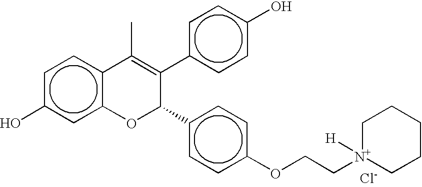

EM-1538, (also called EM-652.HCl) is the hydrochloride salt of the potent antiestrogen EM-652 compared to EM-800, EM-1538 is a simpler and easier salt to synthesize. It was also easy to isolate, purify, crystallizable, and displayed good solid state stability. In administering either EM-800 or EM-1538, it is believed to result in the same active compound in vivo.

Other preferred SERMs of the invention include Tamoxifen ((Z)-2-[4-(1,2-diphenyl-1-butenyl) phenoxy]-N,N-dimethyleth an amine) (available from Zeneca, UK), Toremifene ((Z)-2-[4-(4-Chloro-1,2-diphenyl-1-butenyl)phenoxy]-N,N-dimethylethanamine) available from Orion-Farmos Pharmaceuticla, Finland, or Schering-Plough), Droloxifene ((E)-3-[1-[4-[2-(Dimethylamino) ethoxy]phenyl]-2-phenyl-1-butenyl]phenol) and CP-336,156 (Lasofoxifene) (cis-1R-[4′-pyrrolidino-ethoxyphenyl]-2S-phenyl-6-hydroxy-1,2,3,4,-tetrahydronaphthalene D-(−)-tartrate salt) Pfizer Inc., USA), Raloxifene ([2-(4-hydroxyphenyl)-6-hydroxybenzo[b]thien-3-yl][4-[2-(1-piperidinyl) ethoxy] phenyl]-methanone hydrochloride) (Eli Lilly and Co., USA), LY 335563 (6-hydroxy-3-[4-[2-(1-piperidinyl) ethoxy]phenoxyl]-2-(4-hydroxyphenyl) benzo[b]thiopene hydrochloride) and LY 353381 (Arzoxifene, 6-hydroxy-3-[4-[2-(1-piperidinyl)ethoxy]phenoxyl]-2-(4-methoxyphenyl) benzo [b]thiophene hydrochloride) (Eli Lilly and Co., USA), Idoxifene ((E)-1-[2-[4-[1-(4-Iodophenyl)-2-phenyl-1-butenyl]phenoxy]ethyl]pyrrolidine) (SmithKline Beecham, USA), Levormeloxifene (3,4-trans-2,2-dimethyl-3-phenyl-4-[4-(2-(2-(pyrrolidin-1-yl)ethoxy)phenyl]-7-methoxychroman) (Novo Nordisk, A/S, Denmark) which is disclosed in Shalmi et al. WO 97/25034, WO 97/25035, WO 97/25037, WO 97/25038 ; and Korsgaard et al. WO 97/25036), GW5638 (described by Willson at al., Endocrinology, 138(9), 3901-3911, 1997) and indole derivatives (disclosed by Miller et al. EP 0802183A1) and TSE 424 developed by Wyeth Ayers (USA) and disclosed in JP10036347 (American home products corporation) and nonsteroidal estrogen derivatives described in WO 97/32837. Are also included, Iproxifen (TAT 59; (E)-4-[1-[4-[2-(dimethylamino)ethoxy]phenyl]-2-[4-(1-methylethyl)phenyl]-1-butenyl]phenol dihydrogen phosphate ) from Taiho (Japan), FC 1271 ((Z)-2-[4-(4-chloro-1,2-diphenyl-1-butenyl)phenoxyl]ethanol) from Orion (Finland), HMR 3339 and HMR 3656 from Hoechst Marion Roussel, SH 646 from Schering AG, Germany, ERA 923 from Wyeth Ayerst (USA), LY 335124 and LY 326315 from Eli Lilly (USA).

Any SERM used as required for efficacy, as recommended by the manufacturer, can be used. Appropriate dosages are known in the art. Any other non steroidal antiestrogen commercially available can be used according to the invention. Any compound having activity similar to SERMs (example: Raloxifene can be used).

SERMs administered in accordance with the invention are preferably administered in a dosage range between 0.01 to 10 mg/kg of body weight per day (preferably 0.05 to 1.0 mg/kg), with 5 mg per day, especially 10 mg per day, in two equally divided doses being preferred for a person of average body weight when orally administered, or in a dosage range between 0.003 to 3.0 mg/kg of body weight per day (preferably 0.015 to 0.3 mg/ml), with 1.5 mg per day, especially 3.0 mg per day, in two equally divided doses being preferred for a person of average body weight when parentally administered (i.e. intramuscular, subcutaneous or percutaneous administration). Preferably the SERMs are administered together with a pharmaceutically acceptable diluent or carrier as described below.

Preferred bisphosphonates of the invention administered as active ingredient in the combination therapy for the treatment of osteoporosis include Alendronate [(4-amino-1-hydroxybutylidene)bis phosphonic acid, disodium salt, hydrate] available from Merck Shape and Dohme under the Tradename of Fosamax, Etidronate [(1-hydroxyethylidene)bis phosphonic acid, 2,2′-iminobis ethanol] available from Procter and Gamble under the Trade names of Didrocal and Didronel, Clodronate [(dichloromethylene)bis phosphonic acid, disodium salt] available from Rhône-Poulenc Rorer under the Trade name of Bonefos or available from Boehringer Mannheim under the Trade name of Ostac and, Pamidronate (3-amino-1-hydroxypropylidene)bis phosphonic acid, disodium salt) available from Geigy under the Tradename of Aredia. Risedronate (1-hydroxy-2-(3-pyridinyl)ethylidene bisphosphonic acid monosodium salt) is under clinical development. Any other bisphosphonates commercially available can be used according to the invention, all at the manufacturers' recommended dosage. Likewise sex steroid precursors may be utilized at dosages recommended in the prior art, preferably at dosages that restore circulating levels to those of healthy males 20-30 years of age or those of premenopausal adult females.

With respect to all of the dosages recommended herein, the attending clinician should monitor individual patient response and adjust dosage accordingly.

### EXAMPLES

### Example 1

In the mammary gland, androgens are formed from the precursor steroid dehydroepiandrosterone (DHEA). Clinical evidence indicates that androgens have inhibitory effects on breast cancer. Estrogens, on the other hand, stimulate the development and growth of breast cancer. We studied the effect of DHEA alone or in combination with the newly described pure antiestrogen, EM-800, on the growth of tumor xenografts formed by the human breast cancer cell line ZR-75-1 in ovariectomized nude mice.

Mice received daily subcutaneous injections of 0.5 μg estrone (an estrogenic hormone) immediately after ovariectomy. EM-800 (15, 50 or 100 μg) was given orally once daily. DHEA was applied twice daily (total dose 0.3, 1.0 or 3.0 mg) to the dorsal skin either alone or in combination with a 15 μg daily oral dose of EM-800. Changes in tumor size in response to the treatments were assessed periodically in relation to the measurements made on the first day. At the end of the experiments, tumors were dissected and weighed.

A 9.4-fold increase in tumor size in 9.5 months was observed in ovariectomized mice receiving estrone alone in comparison with mice not receiving estrone. Administration of 15, 50 or 100 μg EM-800 in estrone-supplemented ovariectomized led to inhibitions of 88%, 93%, and 94% in tumor size, respectively. DHEA, on the other hand, at doses of 0.3, 1.0 or 3.0 mg inhibited terminal tumor weight by 67%, 82%, and 85%, respectively. Comparable inhibitions in tumor size were obtained with a daily 15 μg oral dose of EM-800 with or without different doses of percutaneous DHEA.

DHEA and EM-800 independently suppressed the growth of estrone-stimulated ZR-75-1 mouse xenograft tumors in nude mice. Administration of DHEA at the defined doses does not alter the inhibitory effect of EM-800.

Materials and Methods

ZR-75-1 Cells

ZR-75-1 human breast cancer cells were obtained from the American Type Culture Collection (Rockville, Md.) and routinely cultured as monolayers in RPMI 1640 medium supplemented with 2 mM L-glutamine, 1 mM sodium pyruvate, 100 IU penicillin/ml, 100 μg streptomycin/ml, and 10% fetal bovine serum, under a humidified atmosphere of 95% air/5% CO₂ at 37° C. as described (Poulin and Labrie, Cancer Res. 46: 4933-4937, 1986; Poulin et al., Breast Cancer Res. Treat. 12: 213-225, 1988). Cells were passaged weekly after treatment with 0.05% trypsin:0.02% EDTA (w/v). The cell cultures used for the experiments described in this report were derived from passage 93 of the cell line ZR-75-1.

Animals

Female homozygous Harlan Sprague-Dawley (nu/nu) athymic mice (28- to 42-day-old) were obtained from HSD (Indianapolis, Ind., USA). Mice were housed in vinyl cages with air filter tops in laminar air flow hoods and maintained under pathogen-limited conditions. Cages, bedding, and food were autoclaved before use. Water was autoclaved, acidified to pH 2.8, and provided ad libitum.

Cell Inoculation

Mice were bilaterally ovariectomized (OVX) one week before tumor cell inoculation under anesthesia achieved by intraperitoneal injection of 0.25 ml/animal of Avertin (amylic alcohol: 0.8 g/100 ml 0.9% NaCl; and tribromo ethanol: 2 g/100 ml 0.9% NaCl). 1.5×10⁶ ZR-75-1 cells in logarithmic growth phase were harvested after the treatment of monolayer with 0.05% trypsin/0.02% EDTA (w/v), were suspended in 0.1 ml of culture medium containing 25% Matrigel and were inoculated subcutaneously on both flanks of the animals using a 1 inch-long 20-gauge needle as described previously (Dauvois et al., Cancer Res. 51: 3131-3135, 1991). In order to facilitate growth of the tumors, each animal received daily subcutaneous injection of 10 μg of estradiol (E₂) in vehicle composed of 0.9% NaCl 5% ethanol 1% gelatin for 5 weeks. After appearance of palpable ZR-75-1 tumors, tumor diameter was measured with calipers and mice having tumor diameter between 0.2 and 0.7 cm were selected for this study.

Hormonal Treatment

All animals, except those in the control OVX group, received daily subcutaneous injections of 0.5 μg estrone (E₁) in 0.2 ml of 0.9% NaCl 5% ethanol 1% gelatin. In the indicated groups, DHEA was administered percutaneously twice daily at the doses of 0.3, 1.0 or 3.0 mg/animal applied in a volume of 0.02 ml on the dorsal skin area outside the area of tumor growth. DHEA was dissolved in 50% ethanol 50% propylene glycol. EM-800, ((+)-7-pivaloyloxy-3-(4′-pivaloyloxyphenyl)-4-methyl-2-(4″-(2′″-piperidinoethoxy)phenyl)-2H-benzopyran), was synthesized as described earlier (Gauthier et al., J. Med. Chem. 40: 2117-2122, 1997) in the medicinal chemistry division of the Laboratory of Molecular Endocrinology of the CHUL Research Center. EM-800 was dissolved in 4% (v/v) ethanol 4% (v/v) polyethylene glycol (PEG) 600 1% (w/v) gelatin 0.9% (w/v) NaCl. Animals of the indicated groups received daily oral doses of 15 μg, 50 μg, or 100 μg of EM-800 alone or in combination with DHEA while animals of the OVX group received the vehicle (0.2 ml 4% ethanol 4% PEG 600 1% gelatin 0.9% NaCl) alone. Tumors were measured once a week with Vernier calipers. Two perpendicular diameters in cms (L and W) were recorded and tumor area (cm²) was calculated using the formula: L/2×W/2×π (Dauvois et al., Cancer Res. 51: 3131-3135, 1991). The area measured on the first day of treatment was taken as 100% and changes in tumor size were expressed as percentage of initial tumor area. In case of subcutaneous tumors in general, it is not possible to accurately access three dimensional volume of tumor, therefore, only tumors areas were measured. After 291 days (or 9.5 months) of treatment, the animals were sacrificed.

The categories of responses were evaluated as described (Dauvois et al., Breast Cancer Res. Treat. 14: 299-306, 1989; Dauvois et al., Eur. J. Cancer Clin. Oncol. 25: 891-897, 1989; Labrie et al., Breast Cancer Res. Treat. 33: 237-244, 1995). In short, partial regression corresponds to the tumors that regressed equal to or more than 50% of their original size; stable response refers to tumors that regressed less than 50% of the original size or progressed less than 50% of their original size, while complete regression refers to those tumors that were undetectable at the end of treatment. Progression refers to tumors that progressed more than 50% compared with their original size. At the end of the experiment, all animals were killed by decapitation. Tumors, uterus, and vagina were immediately removed, freed from connective and adipose tissues, and weighed.

Statistical Analysis

Statistical significance of the effects of treatments on tumor size was assessed using an analysis of variance (ANOVA) evaluating the effects due to DHEA, EM-800, and time, and repeated measures in the same animals performed at the initiation and at the end of the treatment (subjects within group factor). The repeated measures at time 0 and after 9.5 months of treatment constitute randomized blocks of animals. The time is thus analyzed as a within-block effect while both treatments are assessed as between-block effects. All interactions between main effects were included in the model. The significance of the treatment factors and of their interactions was analyzed using the subjects within group as the error term. Data were log-transformed. The hypotheses underlying the ANOVA assumed the normality of the residuals and the homogeneity of variance.

A posteriori pairwise comparisons were performed using Fisher's test for least significant difference. Main effects and the interaction of treatments on body weight and organ weight were analyzed using a standard two-way ANOVA with interactions. All ANOVAs were performed using SAS program (SAS Institute, Cary, N.C., USA). Significance of differences were declared using a 2-tailed test with an overall level of 5%.

Categorical data were analyzed with a Kruskall-Wallis test for ordered categorical response variables (complete response, partial response, stable response, and progression of tumor). After overall assessment of a treatment effects, subsets of the results presented in Table 4 were analyzed adjusting the critical p-value for multiple comparisons. The exact p-values were calculated using StatXact program (Cytel, Cambridge, Mass., USA).

Data are expressed as means±standard error of the mean (SEM) of 12 to 15 mice in each group.

Results

As illustrated in FIG. 2A, human ZR-75-1 tumors increased by 9.4-fold over 291 days (9.5 months) in ovariectomized nude mice treated with a daily 0.5 μg subcutaneously administered dose of estrone while in control OVX mice who received the vehicle alone, tumor size was decreased to 36.9% of the initial value during the course of the study.

Treatment with increasing doses of percutaneous DHEA caused a progressive inhibition of El-stimulated ZR-75-1 tumor growth. Inhibitions of 50.4%, 76.8%, and 80.0% were achieved at 9.5 months of treatment with the 0.3 mg, 1.0 mg, and 3.0 mg daily doses per animal of DHEA, respectively (FIG. 2A). In agreement with the decrease in total tumor load, treatment with DHEA led to a marked decrease of the average weight of the tumors remaining at the end of the experiment. In fact, average tumor weight decreased from 1.12±0.26 g in control E₁-supplemented ovariectomized nude mice to 0.37±0.12 g (P=0.005), 0.20±0.06 g (P=0.001), and 0.17 ±0.06 g (P=0.0009) in the groups of animals receiving the daily 0.3, 1.0 and 3.0 mg doses of DHEA, respectively (FIG. 2B).

At the daily doses of 15 μg, 50 μg, and 100 μg, the antiestrogen EM-800 inhibited estrogen-stimulated tumor size by 87.5% (P<0.0001), 93.5% (P<0.0001), and 94.0% (P=0.0003), respectively (FIG. 3A) when compared to the tumor size in control animals at 9.5 months. The tumor size reductions achieved with the three EM-800 doses are not significantly different between each other. As illustrated in FIG. 2B, tumor weight at the end of the 9.5-month study was decreased from 1.12±0.26 g in control E₁-supplemented OVX mice to 0.08±0.03 g, 0.03±0.01 g and 0.04±0.03 g in animals treated with the daily 15 μg, 50 μg, and 100 μg doses of EM-800, respectively (P<0.0001 at all doses of EM-800 vs E₁ supplemented OVX).

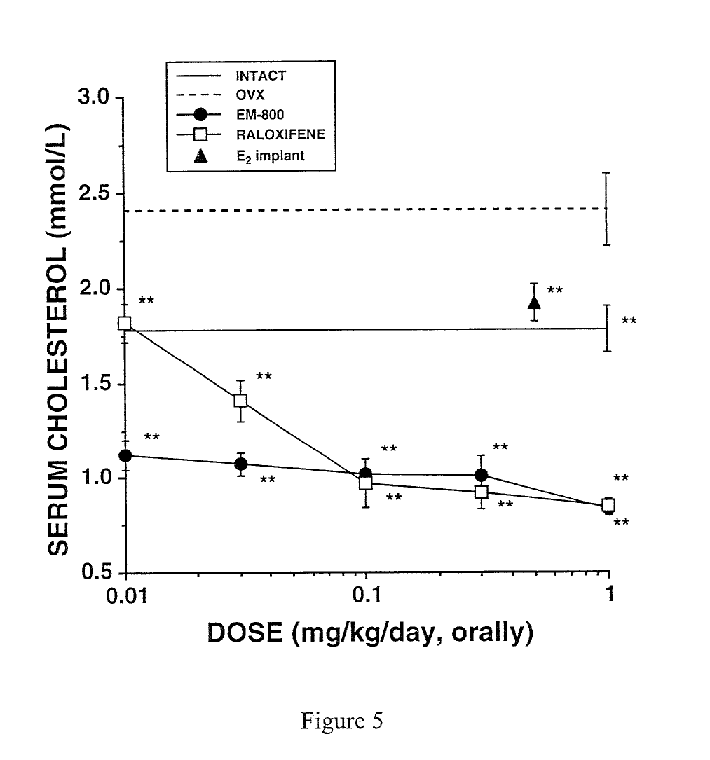

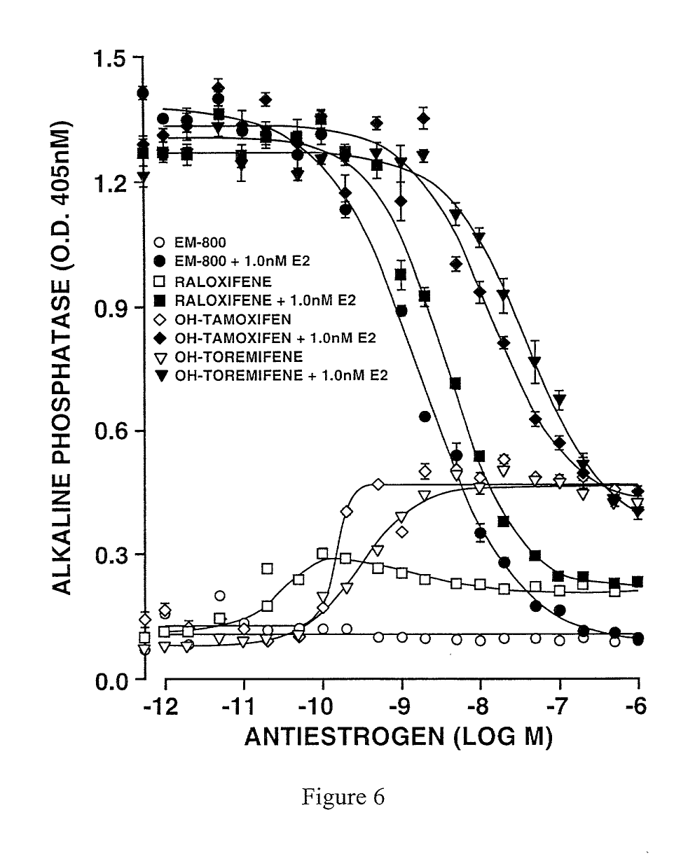

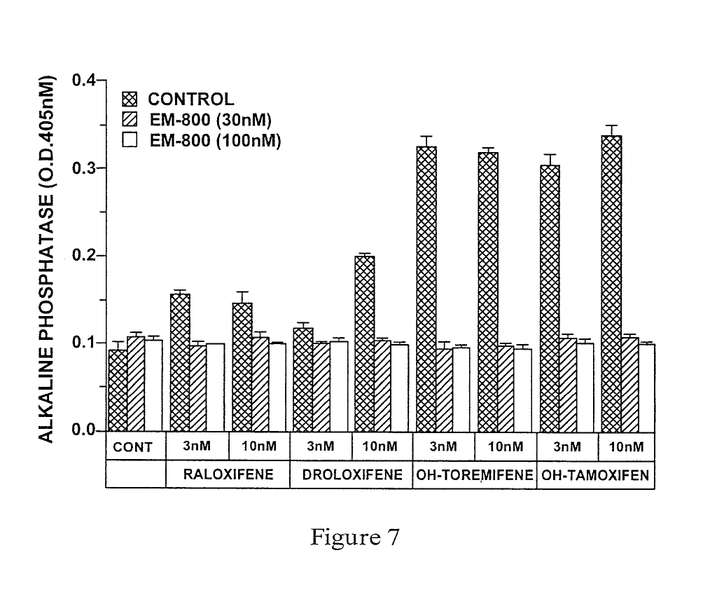

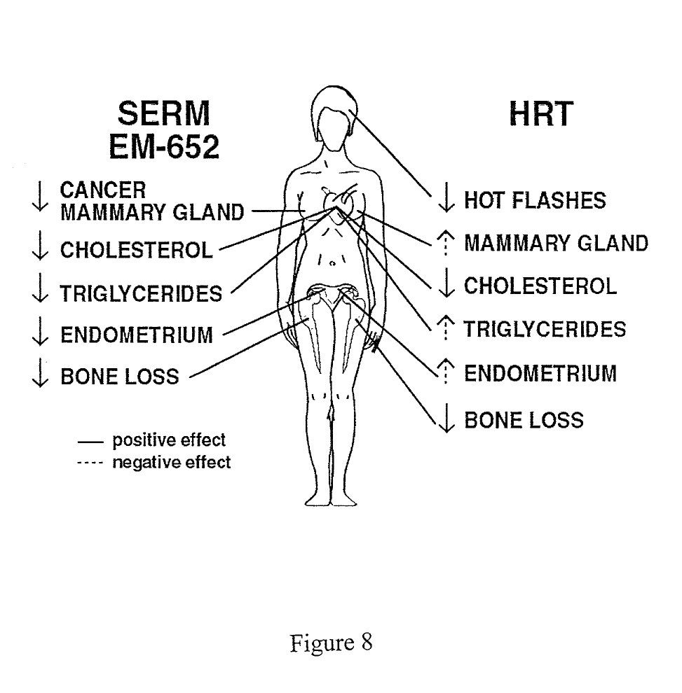

As mentioned above, the antiestrogen EM-800, at the daily oral dose of 15 μg, caused a 87.5% inhibition of estrone-stimulated tumor growth measured at 9.5 months. The addition of DHEA at the three doses used had no significant effect on the already marked inhibition of tumor size achieved with the 15 μg daily dose of the antiestrogen EM-800 (FIG. 5B). Thus, average tumor weight was dramatically reduced from 1.12±0.26 g in control estrone-supplemented mice to 0.08±0.03 g (P<0001), 0.11±0.04 g (P=0.0002), 0.13±0.07 g (P=0.0004) and 0.08±0.05 g ( P<0.0001) in the animals who received the daily dose of 15 μg of the antiestrogen alone or in combination with the 0.3, 1.0, and 3.0 mg doses of DHEA, respectively (no significant difference was noted between the 4 groups) (FIG. 2B).

It was also of interest to examine the categories of responses achieved with the above-indicated treatments. Thus, treatment with the increasing doses of DHEA decreased, although not to a level of statistical significance (P=0.088), the number of progressing tumors from 87.5% in the control OVX animals supplemented with estrone to values of 50.0%, 53.3%, and 66.7% in the animals treated with the daily doses of 0.3, 1.0 or 3.0 mg of DHEA (Table 4). Complete responses, on the other hand, increased from 0% in the estrone-supplemented mice to 28.6%, 26.7%, and 20.0% in the animals receiving the 0.3, 1.0, and 3.0 mg daily doses of percutaneous DHEA. Stable responses, on the other hand, were measured at 12.5%, 21.4%, 20.0%, and 13.3% in the control E₁-supplemented mice and in the three groups of animals who received the above-indicated doses of DHEA, respectively. In control ovariectomized mice, the rates of complete, partial and stable responses were measured at 68.8%, 6.2%, and 18.8%, respectively, while progression was seen in only 6.2% of tumors (Table 2).

Complete responses or disappearance of the tumors were achieved in 29.4%, 33.3%, 26.7%, and 35.3% of tumors in the animals who received the antiestrogen EM-800 (P=0.0006) alone (15 μg) or in combination with the 0.3 mg, 1.0 mg, or 3.0 mg of DHEA, respectively (Table 4). Progression, on the other hand, was seen in 35.3%, 44.4%, 53.3%, and 17.6% of the tumors, in the same groups of animals, respectively. There is no significant difference between the groups treated with EM-800, either alone or in combination with DHEA.

No significant effect of DHEA or EM-800 treatment was observed on body weight adjusted for tumor weight. Treatment of OVX mice with estrone, increased uterine weight from 28±5 mg in OVX control mice to 132±8 mg (P<0.01) while increasing doses of DHEA caused a progressive but relatively small inhibition of the stimulatory effect of estrone which reached 26% (P=0.0008) at the highest dose of DHEA used. It can be seen in the same figure that estrone-stimulated uterine weight was decreased from 132±8 mg in control estrone-supplemented mice to 49±3 mg, 36±2 mg, and 32±1 mg (P<0.0001 at all doses vs control) with the daily oral doses of 15 μg, 50 μg, or 100 μg of EM-800 (overall P<0.0001), respectively. Fifteen micrograms (15 μg) EM-800 in combination with the 0.3 mg, 1.0 mg or 3.0 mg daily doses of DHEA, uterine weight was measured at 46±3 mg, 59±5 mg and 69±3 mg, respectively.

On the other hand, treatment with estrone increased vaginal weight from 14±2 mg in OVX animals to 31±2 mg (P<0.01) while the addition of DHEA had no significant effect. Vaginal weight was then reduced to 23±1 mg, 15±1 mg, and 11±1 mg following treatment with the daily 15 μg, 50 μg or 100 μg doses of EM-800, respectively (overall p and pairwise P<0.0001 at all doses vs control). In combination with the 0.3 mg, 1.0 mg or 3.0 mg doses of DHEA and of EM-800, vaginal weight was measured at 22±1 mg, 25±2 mg and 23±1 mg, respectively (N.S. for all groups versus 15 μg EM-800). It should be mentioned that at the highest dose used, namely 100 μg daily, EM-800 decreased uterine weight in estrone-supplemented OVX animals to a value not different from that of OVX controls while vaginal weight was reduced to a value below that measured in OVX controls (P<0.05). DHEA, probably due to its androgenic effects, partially counteracted the effect of EM-800 on uterine and vaginal weight.

TABLE 2
Effect of percutaneous administration of DHEA or oral administration of
EM-800 alone or in combination for 9.5 months on the responses (complete, partial,
stable, and progression) of human ZR-75-1 breast tumor xenografts in nude mice.

| | | TOTAL | CATEGORY OF RESPONSE | | | |
|---|---|---|---|---|---|---|
| | | NUMBER OF | Complete | Partial | Stable | Progression |
| GROUP | | ANIMALS | Number and (%) | | | |
| OVX | | 16 | 11 | 1 (6.2) | 3 (18.8) | 1 (6.2) |
| | | | (68.8) | | | |
| OVX + E1 (0.5 μg) | | 16 | 0 | 0 (0) | 2 (12.5) | 14 (87.5) |
| | | | (0) | | | |
| OVX + E1 (0.5 μg) + DHEA | 0.3 mg | 14 | 4 | 0 (0) | 3 (21.4) | 7 (50.0) |
| | | | (28.6) | | | |
| | 1.0 mg | 15 | 4 | 0 (0) | 3 (20.0) | 8 (53.3) |
| | | | (26.7) | | | |
| | 3.0 mg | 15 | 3 | 0 (0) | 2 (13.3) | 10 (66.7) |
| | | | (20.0) | | | |
| OVX + E1 (0.5 μg) + EM-800 | 15 μg | 17 | 5 | 1 (5.9) | 5 (29.4) | 6 (35.3) |
| | | | (29.4) | | | |
| | 50 μg | 16 | 4 | 3 (18.8) | 5 (31.2) | 4 (25.0) |
| | | | (25.0) | | | |
| | 100 μg | 16 | 8 | 0 (0) | 3 (18.8) | 5 (31.2) |
| | | | (50.0) | | | |
| OVX + E1 (0.5 μg) + | 0.3 mg | 18 | 6 | 0 (0) | 4 (22.2) | 8 (44.4) |
| | | | (33.3) | | | |
| EM-800 + DHEA | 1.0 mg | 15 | 4 | 0 (0) | 3 (20.0) | 8 (53.3) |
| | | | (26.7) | | | |
| | 3.0 mg | 17 | 6 | 0 (0) | 8 (47.1) | 3 (17.6) |
| | | | (35.3) | | | |

E₁ = Estrone;
DHEA = dehydroepiandrosterone;
OVX = ovariectomized

### Example 2

Androstene-3β,17β-diol (5-diol) possesses intrinsic estrogenic activity. In addition, as a precursor sex steroid, it can be transformed into active androgens and/or other estrogens in peripheral intracrine tissues. In order to assess the relative importance of the androgenic and estrogenic components of 5-diol action on bone mass, twenty-one week old rats were ovariectomized and treated percutaneously once daily with 2, 5, or 12.5 mg of 5-diol alone or in combination with the antiandrogen Flutamide (FLU, 10 mg, s.c., once daily), and/or the antiestrogen EM-800 (100 μg, s.c., once daily) for 12 months. Bone mineral density (BMD) was measured after 11 months of treatment. Ovariectomy (OVX) led to a 12.8% decrease in femoral BMD (p<0.01) while treatment with the highest dose of 5-diol restored 34.3% of femoral BMD lost during the 11 months following OVX (p<0.01). Simultaneous administration of FLU completely prevented the stimulatory effect of 5-diol on femoral BMD while the addition of EM-800 resulted in an additional 28.4% stimulation compared to the effect of 5-diol alone. The simultaneous administration of 5-diol, FLU, and EM-800 only displayed the effect of EM-800 (27%) since the effect of 5-diol was completely blocked by FLU. Comparable results were obtained on BMD of lumbar spine although lumbar spine BMD in OVX rats receiving 12.5 mg 5-diol alone, 12.5 mg 5-diol+EM-800 or 5-diol+FLU+EM-800 was restored to values not significantly different from those of intact animals. The histomorphometric analysis shows that the stimulatory effects of 5-diol on bone volume, trabecular number and the inhibitory effect on trabecular separation of secondary spongiosa of the proximal tibia metaphyseal area are abolished by FLU, but further enhanced by EM-800. The marked stimulation of serum alkaline phosphatase activity obtained following the treatment with 5-diol is 57% (p<0.01 vs 12.5 mg 5-diol alone) reversed by the simultaneous administration of FLU. Treatment with 5-diol had no statistically significant inhibitory effect on the urinary ratio of calcium to creatinine. The highest dose of 5-diol caused a significant 23% (p<0.01) reduction of serum cholesterol while the addition of EM-800 decreased serum cholesterol by 62% (p<0.01). The present data clearly show the stimulatory effect of 5-diol on bone formation and suggest that although 5-diol is a weak estrogen, its stimulatory effect on bone formation is predominantly mediated by an androgenic effect. Moreover, the additive stimulatory effects of EM-800 and 5-diol on bone mass demonstrate the bone-sparing effect of the anti-estrogen EM-800 in the rat. The cholesterol-lowering activity of both 5-diol and EM-800 could have interesting utility for the prevention of cardiovascular diseases.

### Example 3

### Example of Synthesis of the Preferred Compound of the Invention

Synthesis of (S)-(+)-7-hydroxy-3-(4′-hydroxyphenyl)-4-methyl-2-(4″-(2′″-piperidinoethoxy)phenyl)-2H-1-benzopyran hydrochloride EM-01538 (EM-652, HCl)

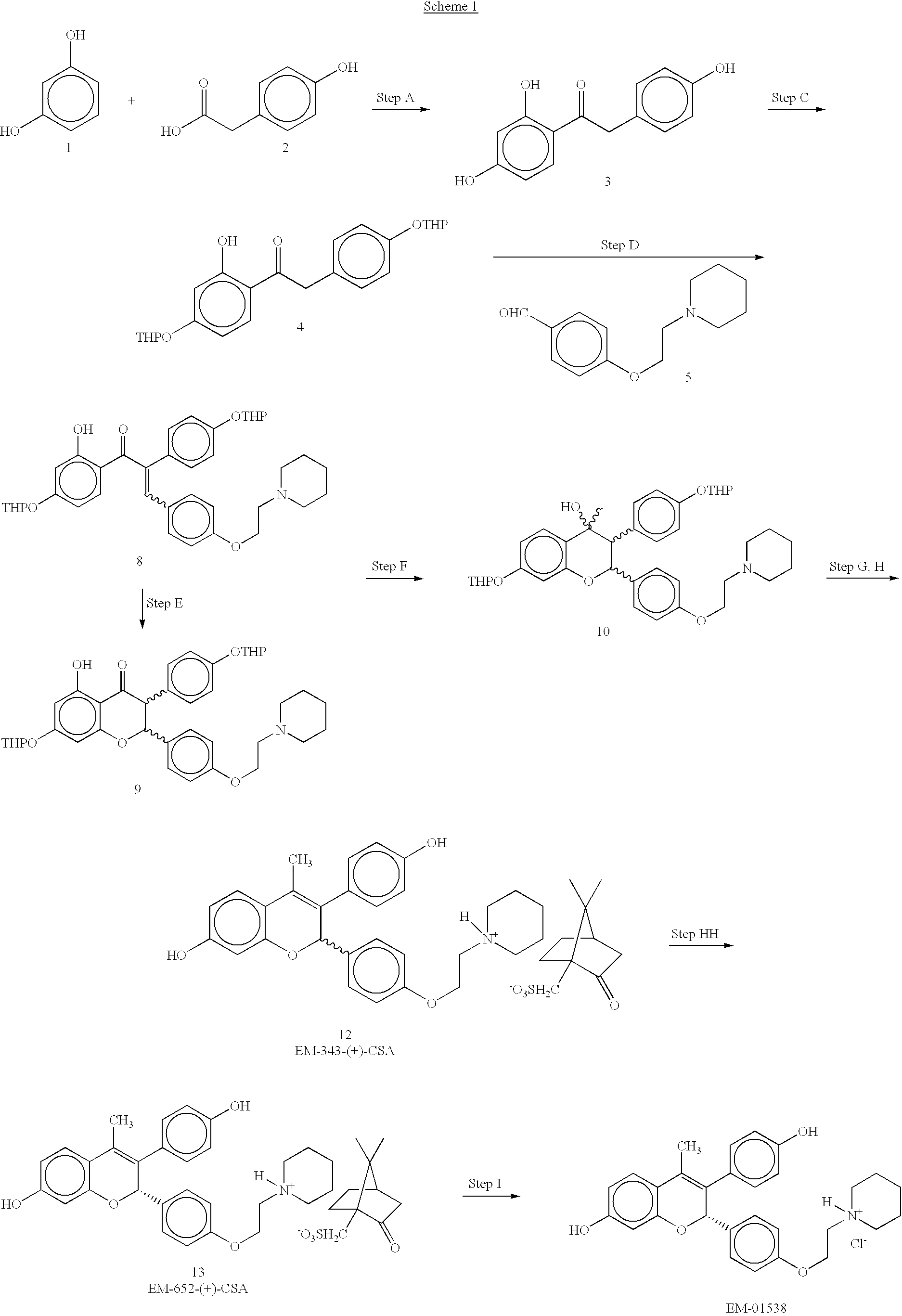

Step A: BF₃Et₂O, toluene; 100° C.; 1 hour.

Step C: 3,4-dihydropyran, p-toluenesulfonic acid monohydrate, ethyl acetate; 25° C. under nitrogen, 16 hours, and then crystallization in isopropanol.

Steps D, E, and F:

(1) piperidine, toluene, Dean & Stark apparatus, reflux under nitrogen; (2) 1,8-diazabicyclo[5, 4, 0]undec-7-ene, DMF, reflux 3 hours; (3) CH₃MgCl, THF, −20 to 0° C. and then room temperature for 24 hours;

Steps G, H: (1S)-(+)-10-camphorsulfonic acid, acetone, water, toluene, room temperature, 48 hours.

Step HH: 95% ethanol, 70° C., then room temperature 3 days.

Step HHR: Recycling of mother liquor and wash of step HH (S)-10-camphorsulfonic acid, reflux; 36 hours, then room temperature for 16 hours.

Step I:

(1) DMF aq., Na₂CO₃, ethyl acetate;

(2) ethanol, dilute HCl;

(3) water.

Synthesis of 2-tetrahydropyranyloxy-4-hydroxy-2′-(4″-tetrahydropyranyloxyphenyl) acetophenone (4). A suspension of 2,4-dihydroxy-2′-(4″-hydroxyphenyl)acetophenone 3 (97.6 g, 0.4 mole) (available from Chemsyn Science Laboratories, Lenexa, Kans.) in 3,4-dihydropyran (218 ml, 3.39 mole) and ethyl acetate (520 ml) was treated with p-toluenesulfonic acid monohydrate (0.03 g, 0.158 mmole) at about 25° C. The reaction mixture was stirred under nitrogen with no external heating for about 16 hours. The mixture was then washed with a solution of sodium bicarbonate (1 g) and sodium chloride (5 g) in water (100 ml). The phases were separated and the organic phase was washed with brine (20 ml). Each wash was back extracted with 50 ml ethyl acetate. All the organic phases were combined and filtered through sodium sulfate.

Solvent (about 600 ml) was removed by distillation at atmospheric pressure and isopropanol (250 ml) was added. Additional solvent (about 300 ml) was distilled at atmospheric pressure and isopropanol (250 ml) was added. Additional solvent (about 275 ml) was distilled at atmospheric pressure and isopropanol (250 ml) was added. The solution was cooled at about 25° C. with stirring and after about 12 hours, the crystalline solid was filtered, washed with isopropanol and dried (116.5 g, 70%).

Synthesis of 4-hydroxy-4-methyl-2-(4′-[2″-piperidino]-ethoxy)phenyl-3-(4′″-tetrahydropyranyloxy)phenyl-7-tetrahydropyranyloxy-chromane (10). A solution of 2-tetrahydropyranyloxy-4-hydroxy-2′-(4″-tetrahydropyranyloxyphenyl)acetophenone 4 (1 kg, 2.42 mole), 4-[2-(1-piperidino)ethoxy]benzaldehyde 5 (594 g, 2.55 mole) (available from Chemsyn Science Laboratories, Lenexa, kans.) and piperidine (82.4 g, 0.97 mole) (available from Aldrich Chemical Company Inc., Milwaukee, Wis.) in toluene (8L) was refluxed under nitrogen with a Dean & Stark apparatus until one equivalent of water (44 mL) was collected.

Toluene (6.5 L) was removed from the solution by distillation at atmospheric pressure. Dimethylformamide (6.5 L) and 1,8-diazabicyclo[5,4,0]undec-7-ene (110.5 g, 0.726 mole) were added. The solution was agitated for about 8 hours at room temperature to isomerize the chalcone 8 to chromanone 9 and then added to a mixture of water and ice (8 L) and toluene (4 L). The phases were separated and the toluene layer washed with water (5 L). The combined aqueous washes were extracted with toluene (3×4 L). The combined toluene extracts were finally washed with brine (3×4 L), concentrated at atmospheric pressure to 5.5 L and then cooled to −10° C.

With continued external cooling and stirring under nitrogen, a 3M solution of methylmagnesium chloride in THF (2.5 L, 7.5 mole) (available from Aldrich Chemical Company Inc., Milwaukee, Wis.) was added, maintaining the temperature below 0° C. After all the Grignard reagent was added, the external cooling was removed and the mixture allowed warm to room temperature. The mixture was stirred at this temperature for about 24 hours.

The mixture was again cooled to about −20° C. and with continued external cooling and stirring, saturated ammonium chloride solution (200 ml) was added slowly, maintaining the temperature below 20° C. The mixture was stirred for 2 hours and then added the saturated ammonium chloride solution (2 L) and toluene (4 L) and agitated for five minutes. The phases were separated and the aqueous layer extracted with toluene (2×4 L). The combined toluene extracts were washed with dilute hydrochloric acid until the solution became homogenous and then with brine (3×4 L). The toluene solution was finally concentrated at atmospheric pressure to 2L. This solution was used directly in the next step.

Synthesis of (2R,S)-7-hydroxy-3-(4′-hydroxyphenyl)-4-methyl-2-(4″-[2′″-piperidino] ethoxy)phenyl)-2H-1-benzopyran (1S)-10-camphorsulphonic acid salt (±12). To the toluene solution of 4-hydroxy-4-methyl-2-(4′-[-2″-piperidino]-ethoxy)-phenyl-3-(4′″-tetrahydropyranyloxy)phenyl-7-tetrahydropyranyloxychromane (10) was added acetone (6 L), water (0.3 L) and (S)-10-camphorsulphonic acid (561 g, 2.42 mole) (available from Aldrich Chemical Company Inc., Milwaukee, Wis.). The mixture was agitated under nitrogen for 48 hours after which time the solid (2R,S)-7-hydroxy-3-(4′-hydroxyphenyl)-4-methyl-2-(4″-[2′″-piperidino]ethoxy)phenyl)-2H-1-benzopyran (1S)-10-camphorsulphonic acid salt (12) was filtered, washed with acetone and dried (883 g). This material was used in the next (HH) step without further purification.

Synthesis of (2S)-7-hydroxy-3-(4′-hydroxyphenyl)-4-methyl-2-(4″-[2′″-piperidino]ethoxy)phenyl)-2H-1-benzopyran (1S)-10-camphorsulphonic acid salt (13, (+)-EM-652(1S)-CSA salt). A suspension of (2R,S)-7-hydroxy-3-(4′-hydroxyphenyl)-4-methyl-2-(4″-[2′″-piperidino]ethoxy)phenyl)-2H-benzopyran (1S)-10-camphorsulphonic acid salt±12 (759 g) in 95% ethanol was heated with stirring to about 70° C. until the solid had dissolved. The solution was allowed to cool to room temperature with stirring then seeded with a few crystals of (2S)-7-hydroxy-3-(4′-hydroxyphenyl)-4-methyl-2-(4″-[2′″-piperidino]ethoxy)phenyl)-2H-1-benzopyran (1S)-10-camphorsulphonic acid salt 13. The solution was stirred at room temperature for about three days in total. The crystals were filtered, washed with 95% ethanol and dried (291 g, 76%). The de of the product was 94.2% and the purity 98.8%.

Synthesis of (S)-(+)-7-hydroxy-3-(4′-hydroxyphenyl)-4-methyl-2-(4″-(2′″-piperidinoethoxy)phenyl)-2H-1-benzopyran hydrochloride EM-01538 (EM-652, HCl). A suspension of compound 13 (EM-652-(+)-CSA salt, 500 mg, 0.726 mmol) in dimethylformamide (11 μL, 0.15 mmol) was treated with an 0.5 M aqueous sodium carbonate solution (7.0 mL, 3.6 mmol), and stirred for 15 min. The suspension was treated with ethyl acetate (7.0 mL) and stirred during 4 h. The organic phase was then washed with an aqueous saturated sodium carbonate solution (2×5 mL) and brine (1×5 mL) dried over magnesium sulfate, and concentrated. A solution of the resulting pink foam (EM-652) in ethanol (2 mL) was treated with 2 N hydrochloric acid (400 μL, 0.80 mmol), stirred for 1 h, treated with distilled water (5 mL), and stirred during 30 min. The resulting suspension was filtered, washed with distilled water (5 mL), dried in air and under high vacuum (65° C.) to give a creamy powder (276 mg, 77%): Fine off-white powder; Scanning Calorimetry: Melting peak onset at 219° C., ΔH=83 J/g; [α]²⁴_{D}=154° in methanol 10 mg/ml.; ¹H NMR (300 MHz, CD₃OD) δ (ppm) 1.6 (broad, 2H, H-4′″), 1.85 (broad, 4H, H-3″″ and 5″″), 2.03 (s, 3H, CH₃), 3.0 and 3.45 (broad, 4H, H-2″″and 6″″), 3.47 (t, J=4.9 Hz, 2H, H-3′″), 4.26 (t, J=4.9 Hz, 2H, H-2′″), 5.82 (s, 1H, H-2), 6.10 (d, J=2.3 Hz, 1H, H-8), 6.35 (dd, J=8.4, 2.43 Hz, 1H, H-6), 6.70 (d, J=8.6 Hz, 2H, H-3′, and H-5′), 6.83 (d, J=8.7 Hz, 2H, H-3″ and H-5″), 7.01 (d, J=8.5 Hz, 2H, H-2′ and H-6′), 7.12 (d, J=8.4 Hz, 1H, H-5), 7.24 (d, J=8.6 Hz, 2H, H-2″ and H-6″); ¹³C RMN (CD₃OD, 75 MHz) δ ppm 14.84, 22.50, 23.99, 54.78, 57.03, 62.97, 81.22, 104.38, 109.11, 115.35, 116.01, 118.68, 125.78, 126.33, 130.26, 130.72, 131.29, 131.59, 134.26, 154.42, 157.56, 158.96, 159.33. Elemental Composition: C, H, N, Cl: Theory; 70.51, 6.53, 2.84, 7.18, %, Found: 70.31, 6.75, 2.65, 6.89%.

### Example 4

Materials and Methods

Animals

Female BALB/c mice (BALB/cAnNCrlBR) weighing 18-20 g were obtained from Charles-River, Inc. (St-Constant, Quebec, Canada) and housed 5 per cage in a temperature (23±1° C.)- and light (12 h light/day, lights on at 7:15)-controlled environment. The mice were fed rodent chow and tap water ad libitum. The animals were ovariectomized (OVX) under Isoflurane anesthesia via bilateral flank incisions and randomly assigned to groups of 10 animals. Ten mice were kept intact as controls.

Treatments

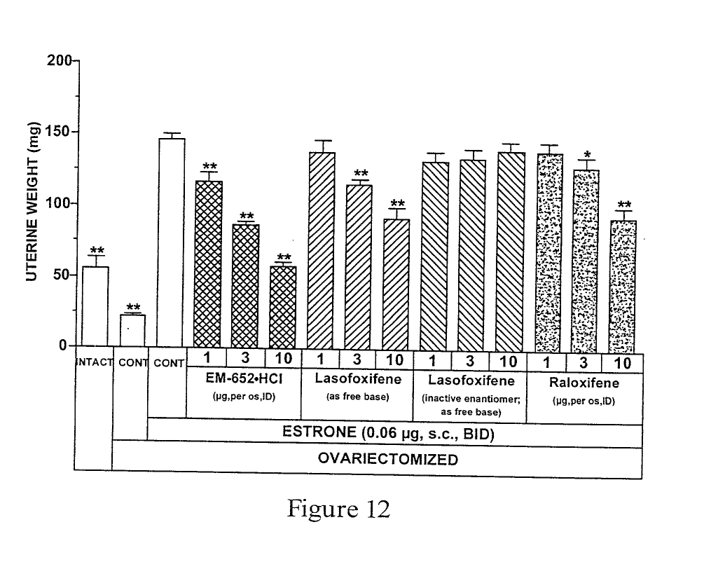

In the first experiment (FIGS. 12 to 15), tested compounds, namely EM-652.HCl, lasofoxifene (as free base; active and inactive enantiomers) and raloxifene, were administered orally by gavage once daily at doses of 1, 3 or 10 μg/animal for 9 days, starting 2 days after ovariectomy. In the second experiment (Table 3), ERA-923 was administered orally by gavage once daily at doses of 1, 3, 10 or 30 μg/animal for 9 days, starting 2 days after ovariectomy. In both experiments, to evaluate the antiestrogenic activity, treatment with estrone (E₁, 0.06 μg, s.c. injection, twice daily) was started 5 days post-ovariectomy and was administered for a 6 day-period. Compounds were dissolved in ethanol (4% final concentration) and administered in 0.4% methylcellulose. Mice in the intact and OVX control groups received the vehicle alone (4% ETOH-0.4% methylcellulose) during the 9-day period. The animals were killed by exsanguination at the abdominal aorta on the 11th morning following ovariectomy. The uteri and vagina were rapidly dissected, weighed, and kept in 10% buffered formalin for further histologic examination.

Results

Experiment 1

As illustrated in FIG. 12, EM-652.HCl administered at the daily oral doses of 1 μg, 3 μg, and 10 μg caused respective 24%, 48%, and 72% inhibitions of estrone-stimulated uterine weight (p<0.01 for all doses versus control) while raloxifene administered at the same doses caused respective 6% (NS), 14% (p<0.01) and 43% (p<0.01) inhibitions of this parameter. Lasofoxifene (as free base), on the other hand, had no inhibitory effect at the lowest dose used while it caused respective 25% (p<0.01) and 44% (p<0.01) inhibitions of estrone-stimulated uterine weight at the daily doses of 3 μg and 10 μg. The inactive enantiomer of lasofoxifene exerted no inhibitory effect on this parameter at any dose used.

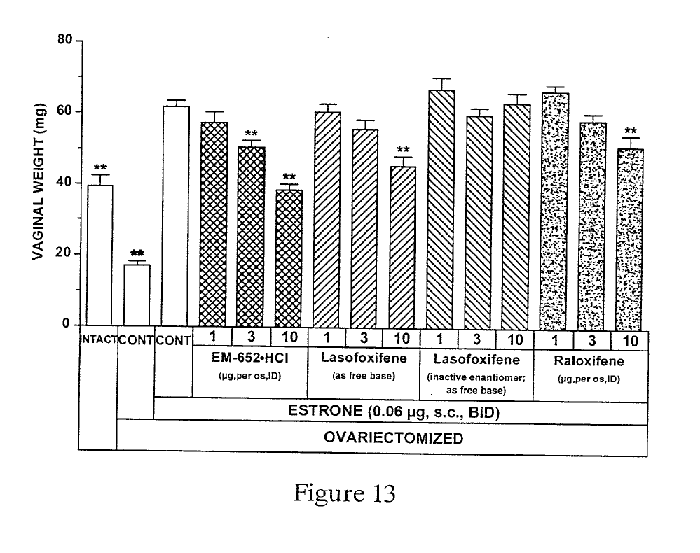

The compounds mentionned above exerted similar effects on vaginal weight. The daily oral administration of EM-652.HCl led to respective 10% (NS), 25% and 53% inhibitions of vaginal weight (p<0.01 for the two highest doses) at the 1 μg, 3 μg, and 10μg doses (FIG. 13), while raloxifene exerted a significant 24% (p<0.01) inhibitory effect on this parameter at the highest dose only (10 μg). Similarly to raloxifene, lasofoxifene (as free base) caused a significant 37% (p<0.01) inhibitory effect only at the highest dose used, while the inactive enantiomer had no inhibitory effect on vaginal weight at any dose used.

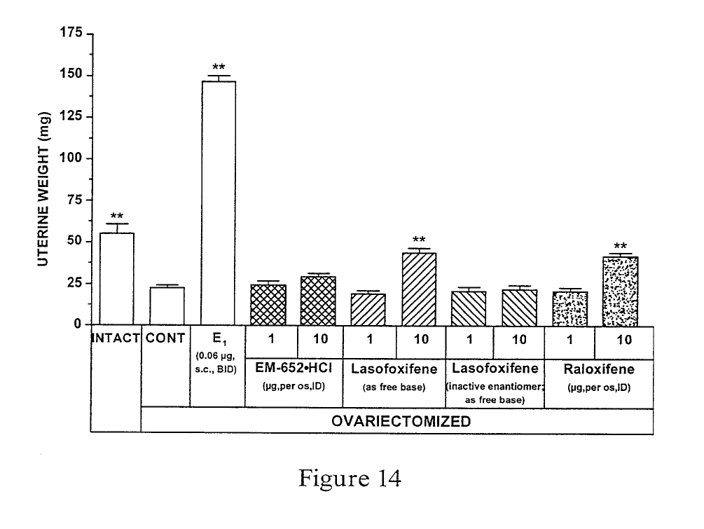

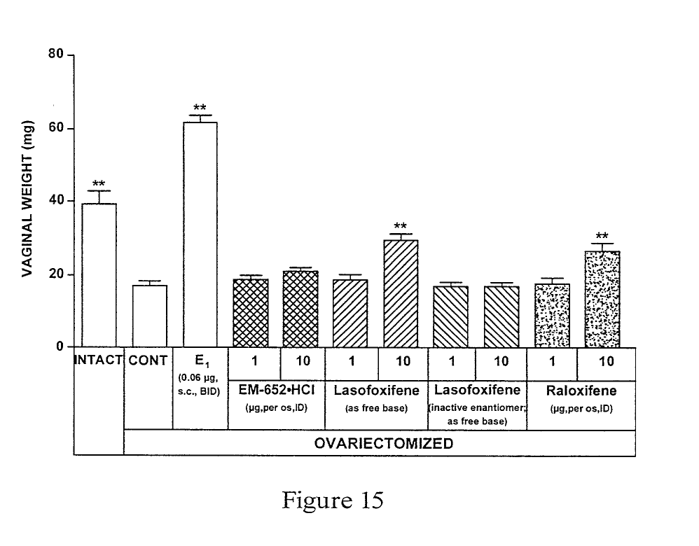

When compounds were administered alone (in the absence of estrone) to ovariectomized mice at the daily oral doses of 1 μg and 10 μg, EM-652.HCl had no significant stimulatory effect on uterine weight at both doses used, while treatment with 10μg of lasofoxifene and raloxifene caused respective 93% (p<0.01) and 85% (p<0.01) stimulations of uterine weight (FIG. 14), thus indicating an estrogenic effect of these latter compounds on this parameter. Similarly, EM-652.HCl exerted no significant stimulatory effect on vaginal weight (FIG. 15) while administration of 10 μg of lasofoxifene and raloxifene caused respective 73% (p<0.01) and 56% (p<0.01) stimulations of vaginal weight. On the other hand, the inactive enantiomer of lasofoxifene had no stimulatory effect on uterine and vaginal weight.

Experiment 2:

As shown in table 3, ERA-923 administered at the daily oral doses of 1 μg, 3 μg, 10 μg or 30 μg caused respective 12% (NS), 47%, 74%, and 94% inhibitions of estrone-stimulated uterine weight (p<0.01 for the three highest doses versus E₁-control). On the other hand, the daily oral administration of ERA-923 led to respective 16% (NS), 56% (p<0.01) and 93% (p<0.01) inhibitions of vaginal weight at the 3 μg, 10 μg, and 30 μg doses.

When the compound was administered alone (in the absence of estrone) to ovariectomized mice at the daily oral doses of 3μg and 30 μg, ERA-923 had no significant stimulatory effect on uterine and vaginal weight at both doses used (Table 3).

TABLE 3
Effect on uterine and vaginal weight of increasing concentrations
of ERA-923 administered orally for 9 days to ovariectomized mice
simultaneously treated or not with estrone.

| | UTERINE WEIGHT | VAGINAL |
|---|---|---|
| TREATMENT | (mg) | WEIGHT (mg) |
| INTACT | 54.6 ± 12.5** | 37.9 ± 3.9** |
| OVX | 15.6 ± 1.3** | 13.9 ± 1.5** |
| OVX + E₁ | 118.3 ± 6.0 | 53.4 ± 2.8 |
| OVX + E₁ + ERA-923 1 μg | 105.5 ± 6.1 | 54.2 ± 3.0 |
| OVX + E₁ + ERA-923 3 μg | 69.7 ± 4.4** | 47.2 ± 1.6 |
| OVX + E₁ + ERA-923 10 μg | 42.1 ± 2.7** | 31.1 ± 2.3** |
| OVX + E₁ + ERA-923 30 μg | 21.7 ± 1.7** | 16.7 ± 1.8** |
| OVX + ERA-923 3 μg | 18.3 ± 1.2 | 14.1 ± 1.2 |
| OVX + ERA-923 30 μg | 17.7 ± 1.6 | 15.3 ± 2.0 |

**p < 0.01 versus E₁-treated control.

### Example 5

5A: Preventive Effects on Bone Loss, Serum Lipids and Total Body Fat.

Animals and Treatment

Ten to twelve week-old female Sprague-Dawley rats (Crl:CD(SD)Br) (Charles River Laboratory, St-Constant, Canada) weighing approximately 220-270 g at start of treatment were used. The animals were acclimatized to the environmental conditions (temperature: 22±3° C.; humidity: 50±20%; 12-h light-12-h dark cycles, lights on at 07:15 h) for at least 1 week before starting the experiments. The animals were housed individually and were allowed free access to tap water and a pelleted certified rodent feed (Lab Diet 5002, Ralston Purina, St-Louis, Mo.). Experiments were conducted in an animal facility approved by the Canadian Council on Animal Care (CCAC) and the Association for Assessment and Accreditation of Laboratory Animal Care (AAALAC) in accordance with the CCAC Guide for Care and Use of Experimental Animals.

In a first experiment, one hundred fifty-four rats were randomly distributed between 11 groups of 14 animals each as follows: 1) Intact control; 2) OVX control; 3) OVX+E₂ (1 mg/kg); 4) OVX+EM-652.HCl (2.5 mg/kg); 5) OVX+E₂+EM-652.HCl; 6) OVX+dehydroepiandrosterone (DHEA; 80 mg/kg); 7) OVX+DHEA+EM-652.HCl; 8) OVX+DHEA+E₂; 9) OVX+DHEA+E₂+EM-652.HCl; 10) OVX+GW 5638; 11) OVX+E₂+GW 5638. On day 1 of the study, the animals of the appropriate groups were bilaterally ovariectomized (OVX) under isoflurane anesthesia. The DHEA was applied topically on the dorsal skin as a solution in 50% ethanol-50% propylene glycol while the other tested compounds were administered as suspension in 0.4% methylcellulose by oral gavage. Treatments were initiated on day 2 of the study and were performed once daily during 3 months.

In the second experiment, one hundred thirty-two rats were randomly distributed between 9 groups of 14 or 15 animals each as follows: 1) Intact control; 2) OVX control; 3) OVX+Premarin (0.25 mg/kg); 4) OVX+EM-652.HCl (2.5 mg/kg); 5) OVX+Premarin+EM-652.HCl; 6) OVX+ERA-923 (2.5 mg/kg); 7) OVX+Premarin+ERA-923; 8) OVX+lasofoxifene (tartrate salt; racemate; 2.5 mg/kg); 9) OVX+Premarin+lasofoxifene. On day 1 of the study, the animals of the appropriate groups were bilaterally OVX under isoflurane anesthesia. Tested compounds were administered as suspension in 0.4% methylcellulose by oral gavage. Treatments were initiated on day 2 of the study and were performed once daily during 26 weeks. In both experiments, animals not receiving a test article were treated with the appropriate vehicle alone during the same period.

Bone Mineral Density Measurements

After 3 months (experiment 1) or 26 weeks (experiment 2) of treatment, individual rats under Isoflurane anesthesia had their whole body skeleton and lumbar spine scanned using dual energy x-ray absorptiometry (DEXA; QDR 4500A, Hologic, Waltham, Mass.) and a Regional High Resolution Scan software. The bone mineral density (BMD) of the lumbar spine (vertebrae L2 to L4) and the total body composition (fat percentage) were determined.

Serum Assays

After 3 months (experiment 1) or 26 weeks (experiment 2) of treatment, blood samples were collected at the jugular vein from overnight fasted animals (under Isoflurane anesthesia). Samples were processed for serum preparation and frozen at −80° C. until assay. Serum cholesterol levels and alkaline phospatase activity (ALP) were determined using the Boehringer Mannheim Diagnostic Hitachi 911 Analyzer (Boehringer Mannheim Diagnostic Laboratory Systems).

Statistical Analyses

Data are expressed as means±SEM. Statistical significance was determined according to the multiple-range test of Duncan-Kramer (Kramer CY; Biometrics 1956;12:307-310).

Results

As shown in table 4, after 3 months of ovariectomy, BMD of the lumbar spine was 10% lower in OVX control animals than in intact controls (p<0.01). At the doses used, the administration of estradiol and EM-652.HCl alone prevented lumbar spine BMD loss by 98% (p<0.01) and 65% (p<0.05), respectively, while the combined treatment with E₂ and EM-652.HCl prevented the OVX-induced decrease in lumbar spine BMD by 61% (p<0.05). On the other hand, while the administration of DHEA alone prevented lumbar spine BMD by 43% (p<0.05), the combined treatment with DHEA+E₂+EM-652.HCl prevented the OVX-induced decrease in lumbar spine BMD by 91% and led to BMD value not different from intact controls.

In table 5, 26 weeks after ovariectomy, BMD of the lumbar spine was 18% lowered compared to intact controls (p<0.01). The administration of Premarin, EM-652.HCl, ERA-923 and lasofoxifene alone prevented lumbar spine BMD by 54%, 62%, 49% and 61%, respectively (all p<0.01 versus OVX controls). The addition of Premarin to EM-652.HCl, ERA-923 or lasofoxifene led to lumbar spine BMD values not significantly different from those obtained with the administration of each SERM alone (Table 5). Similarly, the addition of DHEA to E₂ or to EM-652.HCl completely prevented the OVX-induced decrease in lumbar spine BMD (Table 4). The positive effect of DHEA on BMD is also supported by its effect on serum alkaline phosphatase activity (ALP), a marker of bone formation and turnover. ALP activity was increased from 73±6 IU/L in OVX control animals to 224±18 IU/L, 290±27 IU/L, 123±8 IU/L and 261±20 IU/L (all p<0.01) in DHEA-, DHEA+EM-652.HCl-, DHEA+E₂- and DHEA+E₂+EM-652.HCl-treated animals, respectively, thus suggesting a stimulatory effect of DHEA on bone formation (Table 6).

In addition to the preventive effects on bone loss, the administration of EM-652.HCl, ERA-923, lasofoxifene, GW 5638, DHEA and E₂ exerts some beneficial effects on total body fat percentage and serum lipids. After three months of ovariectomy, total body fat was increase by 22% (p<0.05; Table 6). The administration of EM-652.HCl completely prevented the OVX-induced fat percentage increase while the addition of DHEA and/or E₂ to the SERM led to fat percentage values below those observed in intact control animals. After 26 weeks of ovariectomy, the 40% fat increase induced by estrogen deficiency was reversed by 74%, 78%, 75% and 114% following the administration of Premarin, EM-652.HCl, ERA-923 or lasofoxifene, respectively, while the addition of Premarin to each SERM completely prevented the OVX-induced fat percentage increase (Table 7).

As shown in Table 6, three months after ovariectomy, a 22% increase in serum cholesterol levels was observed in OVX control rats compared to intact controls (p<0.01). In fact, serum cholesterol was increased from 2.01±0.11 mmol/L in intact animals to 2.46±0.08 mmol/L in OVX controls. The administration of E₂ or DHEA alone decrease serum cholesterol levels to 1.37±0.18 mmol/L and 1.59±0.10 mmol/L, respectively, while the administration of EM-652.HCl alone or in combination with E₂ and/or DHEA led to cholesterol levels significantly lower (between 0.65 to 0.96 mmol/L) than those found in intact animals (2.01±0.11 mmol/L). Similarly, the administration of GW 5638, ERA-923 and lasofoxifene alone or in combination with E₂ or Premarin completely prevented the OVX-induced increase on serum cholesterol levels and led to values lower than those found in intact animals (Tables 6 and 7).

TABLE 4
EFFECT ON PREVENTION OF BONE LOSS FOLLOWING
3 MONTH-TREATMENT WITH ESTRADIOL, EM-652 · HCl,
GW 5638 OR DHEA, ADMINISTERED ALONE OR
IN COMBINATION, TO OVARIECTOMIZED FEMALE RATS

| | LUMBAR SPINE | |
|---|---|---|
| | | Prevention |
| | BMD | of Bone Loss |
| TREATMENT | (g/cm²) | (%) |
| Intact | 0.2461 ± 0.0049** | 100 |
| OVX | 0.2214 ± 0.0044 | — |
| OVX + E₂ | 0.2457 ± 0.0049** | 98 |
| OVX + EM-652 · HCl | 0.2374 ± 0.0027* | 65 |
| OVX + EM-652 · HCl + E₂ | 0.2364 ± 0.0037* | 61 |
| OVX + DHEA | 0.2321 ± 0.0034 | 43 |
| OVX + DHEA + EM-652 · HCl | 0.2458 ± 0.0037** | 99 |
| OVX + DHEA + E₂ | 0.2496 ± 0.0029** | 114 |
| OVX + DHEA + E₂ + EM-652 · HCl | 0.2439 ± 0.0043** | 91 |
| OVX + GW 5638 | 0.2299 ± 0.0060 | 34 |
| OVX + GW 5638 + E₂ | 0.2344 ± 0.0054 | 53 |

*p < 0.05;
**p < 0.01, experimental versus OVX control rats.

TABLE 5
EFFECT ON PREVENTION OF BONE LOSS FOLLOWING
26 WEEK-TREATMENT WITH PREMARIN, EM-652 · HCl,
ERA-923 OR LASOFOXIFENE, ADMINISTERED
ALONE OR IN COMBINATION WITH PREMARIN,
TO OVARIECTOMIZED FEMALE RATS

| | LUMBAR SPINE | |
|---|---|---|
| | | Prevention |
| | BMD | of Bone Loss |
| TREATMENT | (g/cm²) | (%) |
| Intact | 0.2482 ± 0.0067** | 100 |
| OVX | 0.2035 ± 0.0035 | — |
| OVX + Premarin | 0.2277 ± 0.0028** | 54 |
| OVX + EM-652 · HCl | 0.2311 ± 0.0040** | 62 |
| OVX + Premarin + EM-652 · HCl | 0.2319 ± 0.0057** | 64 |
| OVX + ERA-923 | 0.2252 ± 0.0058** | 49 |
| OVX + Premarin + ERA-923 | 0.2223 ± 0.0046** | 42 |
| OVX + Lasofoxifene | 0.2307 ± 0.0040** | 61 |
| OVX + Premarin + Lasofoxifene | 0.2357 ± 0.0035** | 72 |

**p < 0.01, experimental versus OVX control rats.

TABLE 6
EFFECT ON TOTAL BODY FAT PERCENTAGE, SERUM
CHOLESTEROL LEVELS AND ALKALINE PHOSPHATASE
ACTIVITY FOLLOWING 3 MONTH-TREATMENT
WITH ESTRADIOL, EM-652 · HCl, GW 5638 OR
DHEA, ADMINISTERED ALONE OR IN COMBINATION,
TO OVARIECTOMIZED FEMALE RATS

| | TOTAL FAT | CHOLESTEROL | ALP |
|---|---|---|---|
| TREATMENT | (%) | (mmol/L) | (IU/L) |
| Intact | 24.0 ± 1.5* | 2.01 ± 0.11** | 39 ± 2** |
| OVX | 29.2 ± 1.5 | 2.46 ± 0.08 | 73 ± 6 |
| OVX + E₂ | 19.5 ± 2.5** | 1.37 ± 0.18** | 59 ± 4 |
| OVX + EM-652 · HCl | 23.2 ± 1.4** | 0.87 ± 0.04** | 91 ± 6* |
| OVX + EM-652 · HCl + E₂ | 20.4 ± 1.4** | 0.96 ± 0.07** | 92 ± 5* |
| OVX + DHEA | 17.3 ± 1.5** | 1.59 ± 0.10** | 224 ± 18** |
| OVX + DHEA + EM-652 · HCl | 18.0 ± 1.1** | 0.65 ± 0.06** | 290 ± 27** |
| OVX + DHEA + E₂ | 15.8 ± 1.3** | 1.08 ± 0.08** | 123 ± 8** |
| OVX + DHEA + E₂ + EM-652 · HCl | 19.2 ± 1.6** | 0.71 ± 0.08** | 261 ± 20** |
| OVX + GW 5638 | 21.9 ± 1.4** | 1.14 ± 0.08** | 72 ± 6 |
| OVX + GW 5638 + E₂ | 23.2 ± 1.2** | 0.91 ± 0.07** | 80 ± 6 |

*p < 0.05;
**p < 0.01, experimental versus OVX control rats.

TABLE 7
EFFECT ON TOTAL BODY FAT PERCENTAGE, SERUM
CHOLESTEROL LEVELS AND ALKALINE PHOSPHATASE
ACTIVITY FOLLOWING 26 WEEK-TREATMENT WITH
PREMARIN, EM-652 · HCl, ERA-923 OR LASOFOXIFENE,
ADMINISTERED ALONE OR IN COMBINATION WITH
PREMARIN, TO OVARIECTOMIZED FEMALE RATS

| | TOTAL | CHOLESTEROL | ALP |
|---|---|---|---|
| TREATMENT | FAT (%) | (mmol/L) | (IU) |
| Intact | 25.5 ± 1.8** | 2.11 ± 0.11** | 33 ± 2* |
| OVX | 35.7 ± 1.6 | 2.51 ± 0.09 | 60 ± 6 |
| OVX + Premarin | 28.2 ± 1.8** | 1.22 ± 0.07** | 49 ± 3 |
| OVX + EM-652 · HCl | 27.7 ± 1.4** | 0.98 ± 0.06** | 78 ± 4 |
| OVX + EM-652 · HCl + | 25.7 ± 2.2** | 1.10 ± 0.07** | 81 ± 6 |
| Premarin | | | |
| OVX + ERA-923 | 28.0 ± 1.8** | 1.15 ± 0.05** | 85 ± 6 |
| OVX + ERA-923 + | 25.7 ± 1.7** | 1.26 ± 0.14** | 98 ± 22** |
| Premarin | | | |
| OVX + Lasofoxifene | 24.1 ± 1.3** | 0.60 ± 0.02** | 116 ± 9** |
| OVX + Lasofoxifene + | 23.8 ± 1.9** | 0.81 ± 0.12** | 107 ± 6** |
| Premarin | | | |

*p < 0.05;
**p < 0.01, experimental versus OVX control rats.

5B: Treatment Effects on Bone Loss and Total Body Fat

Animals and Treatment

Height to nine month-old female Sprague-Dawley rats (Crl:CD(SD)Br) (Charles River Laboratory, St-Constant, Canada) were used in this experiment. The animals were acclimatized to the environmental conditions (temperature: 22±3° C.; humidity: 50±20%; 12-h light-12-h dark cycles, lights on at 07:15 h) for at least 1 week prior to the ovariectomy. Animals were bilaterally ovariectomized (OVX) under isoflurane anesthesia. Twenty animals were kept intact as control. The animals were housed individually and were allowed free access to tap water and a pelleted certified rodent feed (Lab Diet 5002, Ralston Purina, St-Louis, Mo.). Experiments were conducted in an animal facility approved by the Canadian Council on Animal Care (CCAC) and the Association for Assessment and Accreditation of Laboratory Animal Care (AAALAC) in accordance with the CCAC Guide for Care and Use of Experimental Animals.

Ten weeks after OVX, one hundred thirty-nine rats were randomly distributed between 8 groups of 17 to 20 animals each as follows: 1) Intact control; 2) OVX control; 3) OVX+E₂ (1 mg/kg); 4) OVX+EM-652.HCl (2.5 mg/kg); 5) OVX+E₂+EM-652.HCl; 6) OVX+dehydroepiandrosterone (DHEA; 80 mg/kg); 7) OVX+DHEA+EM-652.HCl; 8) OVX+DHEA+EM-652.HCl+E₂. The DHEA was applied topically on the dorsal skin as a solution in 50% ethanol-50% propylene glycol while E₂ and EM-652.HCl were administered as suspension in 0.4% methylcellulose by oral gavage. Treatments were initiated 10 weeks after the ovariectomy and were performed once daily during 26 weeks. Animals not receiving a test article were treated with the appropriate vehicle alone during the same period.

Bone Mineral Density Measurements

Prior to the OVX, prior to the first day treatment (10 weeks after OVX) and after 26 weeks of treatment, individual rats under Isoflurane anesthesia had their whole body skeleton, lumbar spine and right femur scanned using dual energy x-ray absorptiometry (DEXA; QDR 4500A, Hologic, Waltham, Mass.) and a Regional High Resolution Scan software. The bone mineral density (BMD) of the lumbar spine (vertebrae L2 to L4), femur and the total body composition (fat percentage) were determined.

Statistical Analyses

Data are expressed as means±SEM. Statistical significance was determined according to the multiple-range test of Duncan-Kramer (Kramer CY; Biometrics 1956;12:307-310).

Results

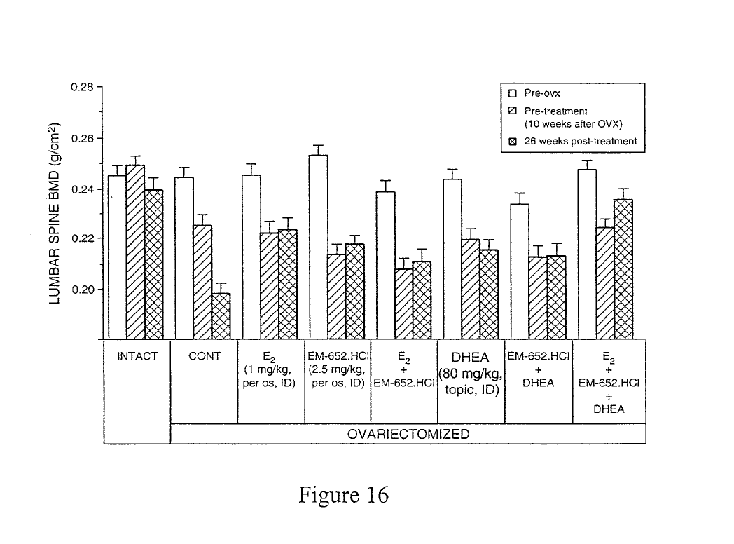

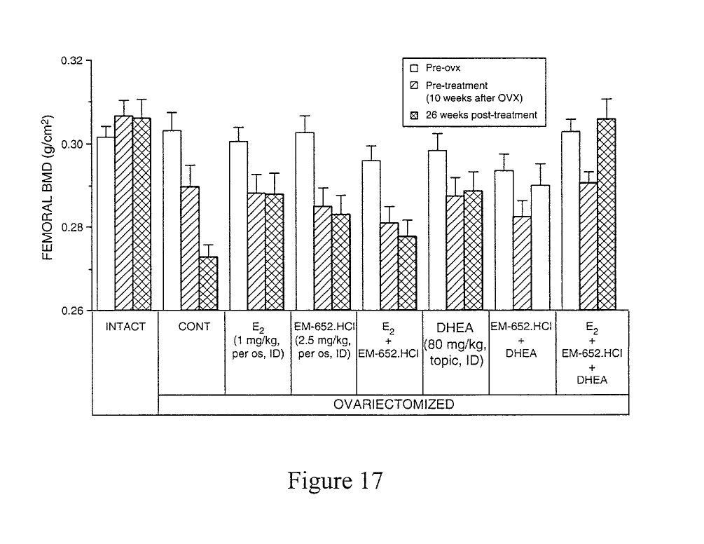

In the previous studies described above (example 5A), the administration of tested compounds was initiated at the time of OVX in order to study the preventive effects on bone loss. In the present study, the administration of tested compounds was initiated 10 weeks after the OVX in order to study the possible curative effects of the treatments administered. The BMD was measured prior to the OVX (baseline values) and prior to the beginning of the treatment in order to establish the presence of osteopenia before initiation of the treatment. As shown in FIG. 16, BMD of the lumbar spine was lowered by 8% after 10 weeks of ovariectomy, and was further decrease by 12% following the additional 26 weeks of OVX during which period the animals received the vehicle alone (control group). The daily administration for 26 weeks of E₂, EM-652.HCl, E₂+EM-652.HCl, DHEA or DHEA+EM-652.HCl to animals having established osteopenia completely prevented the further decrease in lumbar spine BMD observed in OVX control animals, while the administration of E₂+EM-652.HCl+DHEA led to BMD values slightly higher than those observed prior to the start of the treatment. On the other hand, as illustrated in FIG. 17, femoral BMD was lowered by 4% after 10 weeks of ovariectomy, and was further decrease by 6% following the additional 26 weeks of treatment with the vehicle alone. Similar to the lumbar spine BMD, the daily administration for 26 weeks of E₂, EM-652.HCl, E₂+EM-652.HCl, DHEA or DHEA+EM-652.HCl completely prevented the further decrease in femoral BMD observed in OVX control animals. On the other hand, the administration of E₂+EM-652.HCl+DHEA led to femoral BMD values even slightly higher than those observed prior to the OVX thus indicating a beneficial effect of this combined treatment on bone formation. The combined treatment with EM-652.HCl+E₂+DHEA not only completely prevents the further OVX-induced bone loss in animals with osteopenia but also exerts some curative effects.

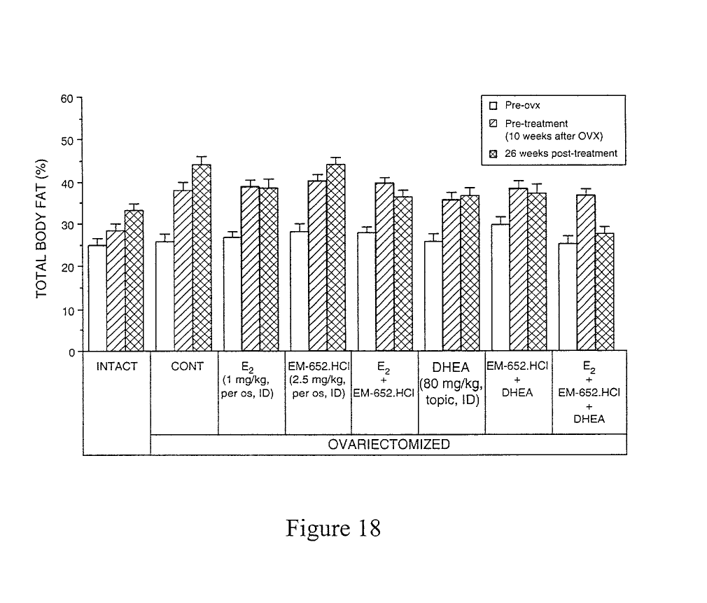

In addition to the effect on bone, as illustrated in FIG. 18, the administration of DHEA, E₂ and/or EM-652.HCl prevented the OVX-induced increase in total body fat. In fact, in OVX control animals, fat percentage was increased by 47% after 10 weeks of ovariectomy, and was further increase by 17% during the 26 weeks of treatment with the vehicle alone (control group). The daily administration of E₂, DHEA, EM-652.HCl, E₂+EM-652.HCl or DHEA+EM-652.HCl for 26 weeks prevented the 17% increase observed in the OVX control group. On the other hand, the combined treatment with EM-652.HCl+E₂+DHEA completely reversed the effect of OVX and led to fat percentage value similar to that found before the ovariectomy of the animals thus indicating a curative effect of this treatment.

### Example 6

### Effect of Compounds of the Invention on Alkaline Phosphatase Activity in Human Endometrial Adenocarcinoma Ishikawa Cells

Materials

Maintenance of Stock Cell Cultures

The human Ishikawa cell line derived from a well differentiated endometrial adenocarcinoma was kindly provided by Dr. Erlio Gurpide, The Mount Sinai Medical Center, New York, N.Y. The Ishikawa cells were routinely maintained in Eagle's Minimum Essential Medium (MEM) containing 5% (vol/vol) FBS (Fetal Bovine Serum) and supplemented with 100 U/ml penicillin, 100 μg/ml streptomycin, 0.1 mM non-essential amino acids solution. Cells were plated in Falcon T75 flasks at a density of 1.5×10⁶ cells at 37° C.

Cell Culture Experiments

Twenty four hours before the start of the experiment, the medium of near confluent Ishikawa cells was replaced by fresh estrogen-free basal medium (EFBM) consisting of a 1:1 (v:v) mixture of phenol red-free Ham's F-12 and Dulbecco's Modified Eagle's Medium (DMEM) supplemented with 100U/mL penicillin, 100 μg/mL streptomycin, 2 mM glutamine, and 5% FBS treated twice with dextran-coated charcoal to remove endogenous steroids. Cells were then harvested by 0.1% pancreatin (Sigma) and 0.25 mM HEPES, resuspended in EFBM and plated in Falcon 96, well flat-bottomed microtiter plates at a density of 2.2×10⁴ cells/well in a volume of 100 μl and allowed to adhere to the surface of the plates for 24 h. Thereafter, medium was replaced with fresh EFBM containing the indicated concentrations of compounds in a final volume of 200 μl. Cells were incubated for five days, with a medium change after 48 h.

Alkaline Phosphatase Assay

At the end of the incubation period, microtiter plates were inverted and growth medium was decanted. The plates were rinsed with 200 μl by well of PBS (0.15M NaCl, 10 mM sodium phosphate, pH 7.4). PBS was then removed from the plates while carefully leaving some residual PBS, and the wash procedure was repeated once. The buffered saline was then decanted, and the inverted plates were blotted gently on a paper towel. Following replacement of the covers, the plates were placed at −80° C. for 15 min followed by thawing at room temperature for 10 min. The plates were then placed on ice, and 50 μl of an ice-cold solution containing 5 mM p-nitrophenyl phosphate, 0.24 mM MgCl₂, and 1 M diethanolamine (pH 9.8) were added. Plates were then warmed to room temperature, and the yellow color from the production of p-nitrophenyl was allowed to develop (8 min). Plates were monitored at 405 nm in an enzyme-linked immunosorbent assay plate reader (BIO-RAD, model 2550 EIA Reader).

Calculations

Dose-response curves as well as IC₅₀ values were calculated using a weighted iterative nonlinear squares regression.

TABLE 8

| | | | Maximal | Inhibition of | Maximal |
|---|---|---|---|---|---|
| | | | stimulation | 1nM E₂- | inhibition |
| | | | of | induced | of 1nM |
| | | | alkaline | stimulation of | E₂-induced |
| | | | phosphatase | alkaline | stimulation of |
| | | | % of InM E₂ | phosphatase | alkaline |
| | | | stimulation * | IC₅₀ (nM) | phosphatase |
| | | | (nb of | (nb of | (nb of |
| NAME | CODE NAME | STRUCTURE | experiments) | experiments) | experiments) |
| EM-652.HCl | EM-652.HCl; (EM-1538) | 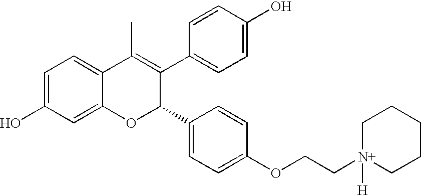 | 1.88 ± 0.26 (22) | 1.52 ± 0.22 (18) | 98.97 ± 0.174 (18) |
| OH- Toremifene | EM-880 | 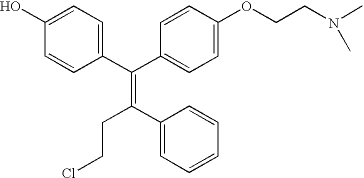 | 29.6 ± 2.1 (6) | 72.1 ± 7.6 (3) | 75.73 ± 3.52 (3) |
| GW-5638 | EM-1796 | 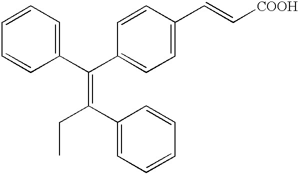 | 7.75 ± 5.5 (2) | No inhibition | |
| Raloxifene LY 156758 | EM-1105 | 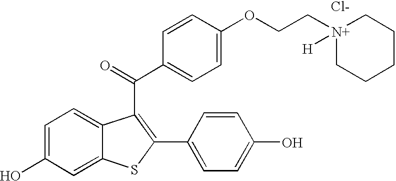 | 12.8 ± 1.7 (8) | 3.39 ± 0.9 (6) | 94.31 ± 1.74 (5) |
| LY 353381 | EM-1665 | 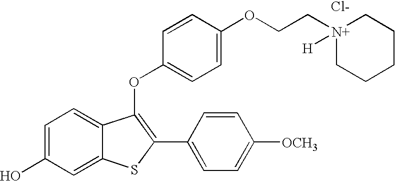 | 15.5 ± 0.25 (5) | 1.87 ± 0.07 (2) | 90.25 ± 0.127 (2) |
| Lasofoxifene (free base) | EM-3114 | 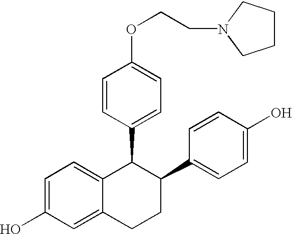 | 17.9 (1) | 4.24 (1) | 85.14 (1) |
| ERA-923 | EM-3527 | 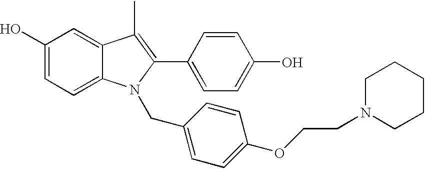 | 0.6 (1) | 5.84 (1) | 100.16 (1) |

*% of 1nM E₂ stimulation = OD 405 nm compound-OD 405 nm basal/OD 405 nm 1nM E₂-OD 405 nm basal
Please see also Labrie et al. EM-652 (SCH 57068), a third generation SERM acting as pure antiestrogen in the mammary gland and endometrium, J. Steroid Biochem. and Mol. Bio. 69, 51-84, 1999.

### Example 7

### Effect of EM-652.HCl, ERA-923, and Lasofoxifene on the Proliferation of Human Breast Cancer MCF-7 Cells

Methods:

Maintenance of Stock Cell Cultures

MCF-7 human breast cancer cells were obtained from the American Type Culture Collection # HTB 22 at passage 147 and routinely grown in phenol red-free Dulbecco's Modified Eagle's-Ham's F12 medium, the supplements mentioned above and 5% FBS. The MCF-7 human breast adenocarcinoma cell line was derived from the pleural effusion of a Caucasian 69-year-old female patient. MCF-7 cells were used between passages 148 and 165 and subcultured weekly

Cell Proliferation Studies

Cells in their late logarithmic growth phase were harvested with 0.1% pancreatin (Sigma) and resuspended in the appropriate medium containing 50 ng bovine insulin/ml and 5% (v/v) FBS treated twice with dextran-coated charcoal to remove endogenous steroids. Cells were plated in 24-well Falcon plastic culture plates (2 cm²/well) at the indicated density and allowed to adhere to the surface of the plates for 72 h. Thereafter, medium was replaced with fresh medium containing the indicated concentrations of compounds diluted from 1000×stock solutions in 99% redistilled ethanol in the presence or absence of E₂. Control cells received only the ethanolic vehicle (0.1% EtOH,v/v). Cells were incubated for the specified time intervals with medium changes at 2-or 3-day intervals. Cell number was determined by measurement of DNA content.

Calculations and Statistical Analysis

Dose-response curves as well IC₅₀ values were calculated using a weighted iterative nonlinear least-squares regression. All results are expressed as means±SEM.

TABLE 9
Experiment 1

| | | Maximal | |
|---|---|---|---|
| | | stimulation | Inhibition of 1 nM |
| | | of DNA by tested | E₂ stimulation |
| | | compounds | of DNA by |
| | CODE | % of 1 nM E₂ | tested compounds |
| NAME | NAME | stimulation* | IC₅₀ (nM) |
| EM-652 · HCl | EM-652 · HCl; | N.S. | 0.796 |
| | EM-1538 | | |
| ERA-923 | EM-3527 | N.S. | 3.68 |

Experiment 2

| | | Stimulation | |
|---|---|---|---|
| | | of DNA | Inhibition of 1 nM |
| | | by tested | E_{2 stimulation} |
| | | compounds | of DNA by |
| | CODE | % of 1 nM E₂ | tested compounds |
| NAME | NAME | stimulation* | IC₅₀ (nM) |
| EM-652 · HCl | EM-652 · HCl; | N.S. | 0.205 |
| | EM-1538 | | |
| Lasofoxifene | EM-3114 | N.S. | 0.379 |
| (free base) | | | |

### Example 8

### Comparison of the Effects of EM-652.HCl, Tamoxifen, Toremifene, Droloxifene, Idoxifene, GW-5638, and Raloxifene on the Growth of Human RZ-75-1 Breast Tumors in Nude Mice.

The objective of this example was to compare the agonistic and antagonistic effects of EM-652.HCl and six other oral antiestrogens (SERMs) on the growth of the well-characterized estrogen-sensitive ZR-75-1 breast cancer xenografts in ovariectomized nude mice.

Materials and Methods

Human ZR-75-1 Breast Cancer Cells

ZR-75-1 human breast cancer cells were obtained from the American Type Culture Collection (Rockville, Md.) and cultured in phenol red-free RPMI-1640 medium. The cells were supplemented with 2 mM L-glutamine, 1 mM sodium pyruvate, 100 IU penicillin/ml, 100 μg streptomycin/ml, and 10% (v/v) fetal bovine serum and incubated under an humidified atmosphere of 95% air/5% CO2 at 37° C. Cells were passaged weekly and harvested at 85-90% confluence using 0.083% pancreatin/0.3 mM EDTA.

Animals and Tumor Inoculation

Homozygous female nu/nu Br athymic mice (28- to 42-day old) were obtained from Charles River, Inc. (Saint-Constant, Québec, Canada). The mice (5 per cage) were housed in vinyl cages equipped with air filter lids, which were kept in laminar airflow hoods and maintained under pathogen-limiting conditions. The photoperiod was 12 hours of light and 12 hours of darkness (lights on at 07:15). Cages, bedding and food (Agway Pro-Lab R-M-H Diet #4018) were autoclaved before use. Water was autoclaved and provided ad libitum. Bilateral ovariectomy was performed under isoflurane-induced anesthesia. At the time of ovariectomy, an implant of estradiol (E₂) was inserted subcutaneously to stimulate initial tumor growth. E₂ implants were prepared in 1 cm-long Silastic tubing (inside diameter: 0.062 inch; outside diameter: 0.095 inch) containing 0.5 cm of a 1:10 (w/w) mixture of estradiol and cholesterol. One week after ovariectomy, 2×10 6 ZR-75-1 (passage 93) cells were inoculated subcutaneously in 0.1 ml of RPMI-1640 medium +30% Matrigel on both flanks of each ovariectomized (OVX) mouse through a 2.5-cm-long 22-gauge needle. After four weeks, the E₂ implants were replaced in all animals by estrone-containing implants of the same size (E1:chol, 1:25, w:w). Randomization and treatments were started one week later.

Treatments

One day prior to initiation of treatments, 255 mice bearing ZR-75-1 tumors of an average area of 24,4±0,4 mm2 (range 5,7 to 50,7 mm²) were randomly assigned to 17 groups (with respect to tumor size), each containing 15 mice (total of 29 or 30 tumors). The 17 groups included two control groups (OVX and OVX+Estrone), seven groups supplemented with an estrone implant and treated with an antiestrogen and eight other groups that received an antiestrogen alone. The estrone implants were then removed from the animals in the ovariectomized control group (OVX) and in groups that were to receive the antiestrogen alone. Estrone-containing implants in the nine other groups were changed thereafter every 6 weeks. EM-652.HCl, raloxifene, droloxifene, idoxifene and GW 5638 were synthesized in the medicinal chemistry division of the Oncology and Molecular Endocrinology Research Center. Tamoxifen was purchased from Plantex (Netanya, Israël) while toremifene citrate was purchased from Orion (Espoo, Finland). Under estrone stimulation, the antiestrogens were given at the daily oral dose of 50 μg (2 mg/kg, on average) suspended in 0.2 ml of 0.4% (w/v) methylcellulose. In the absence of estrone stimulation, animals were treated with 200 μg (8 mg/kg on average) of each antiestrogen once daily by the oral route. Animals in both control groups received 0.2 ml of the vehicle alone. The antiestrogen suspensions at the appropriate concentration were prepared each month, stored at 4° C. and used under constant agitation. Powder stock were hermetically stored at 4° C. (idoxifene, raloxifene, toremifene, GW 5638, droloxifene) or at room temperature (tamoxifen, EM-652•HCl).

Tumor Measurements and Necropsy

Two perpendicular diameters were recorded and tumor area (mm2) was calculated using the formula: L/2×W/2×π. The area measured on the first day of treatment was taken as 100%.

After 161 days of treatment, the remaining animals were anesthetized with isoflurane and killed by exsanguination. To further characterize the effect of the estrogen and antiestrogens, estrogen-responsive tissues, such as the uterus and vagina, were immediately removed, freed from connective and adipose tissue and weighed. The uteri were prepared to evaluate endometrial thickness by image analysis performed with Image Pro-Plus(Media Cybernetics, Maryland, USA). In brief, uteri were fixed in 10% formalin and embedded in parafin. Hematoxylin- and eosin-stained sections of mice uteri were analysed. Four images per uterus (2 per uterine horn) were analyzed. Mean epithelial cell height was measured in all animals of each group.

Response Criteria

Tumor response was assessed at the end of the study or at death of each animal, if it occurred during the course of the experiment. In this case, only data of mice that survived for at least half of the study (84 days) were used in the tumor response analysis. In brief, complete regression identifies those tumors that were undetectable at the end of the experiment; partial regression corresponds to the tumors that regressed≧50% of their original size; stable response refers to tumors that regressed<50% or progressed≦50%; and progression refers to tumors that progressed≧50% compared with their original size.

Statistical Analyses

The change in total tumors surface areas between day 1 and day 161 were analyzed according to an ANOVA for repeated measurements. The model included the treatment, time, and time-treatment interaction effects plus the term to account for the strata at randomization. The significance of the different treatments effects at 161 days was thus tested by the time-treatment interaction. Analysis of the residuals indicated that the measurements on the original scale were not fitted for analysis by an ANOVA nor any of the transformations that were tried. The ranks were therefore selected for the analyses. The effect of the treatments on the epithelial thickness was assessed by a one-way ANOVA including also the strata at randomization. A posteriori pairwise comparisons were performed using least square means statistics. The overvall type 1 error rate (α) was controlled at 5% to declare significance of the differences. All calculations were performed using Proc MIXED on the SAS Software (SAS Institute, Carry, N.C.).

Results

Antagonistic Effects on ZR-75-1 Tumor Growth

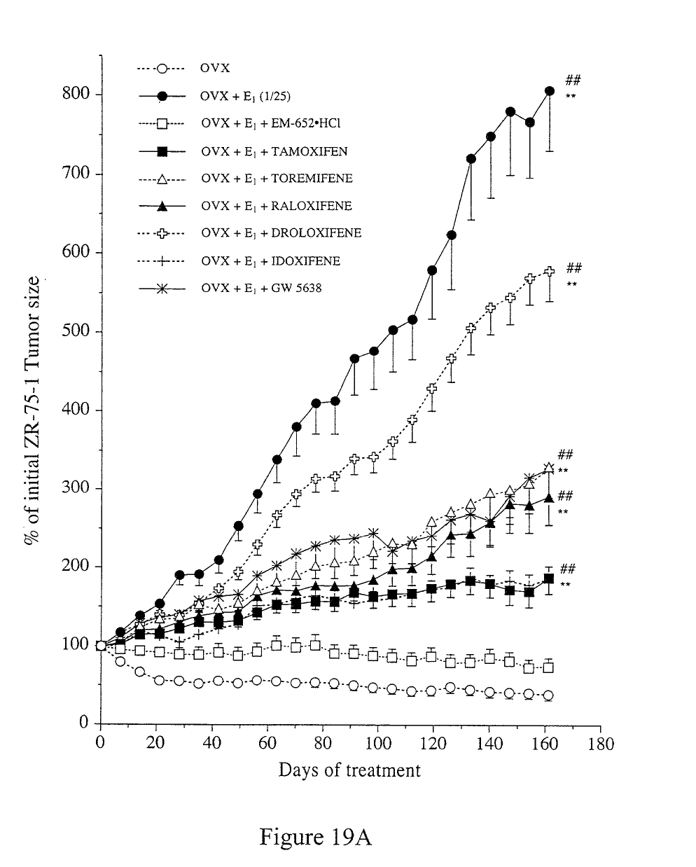

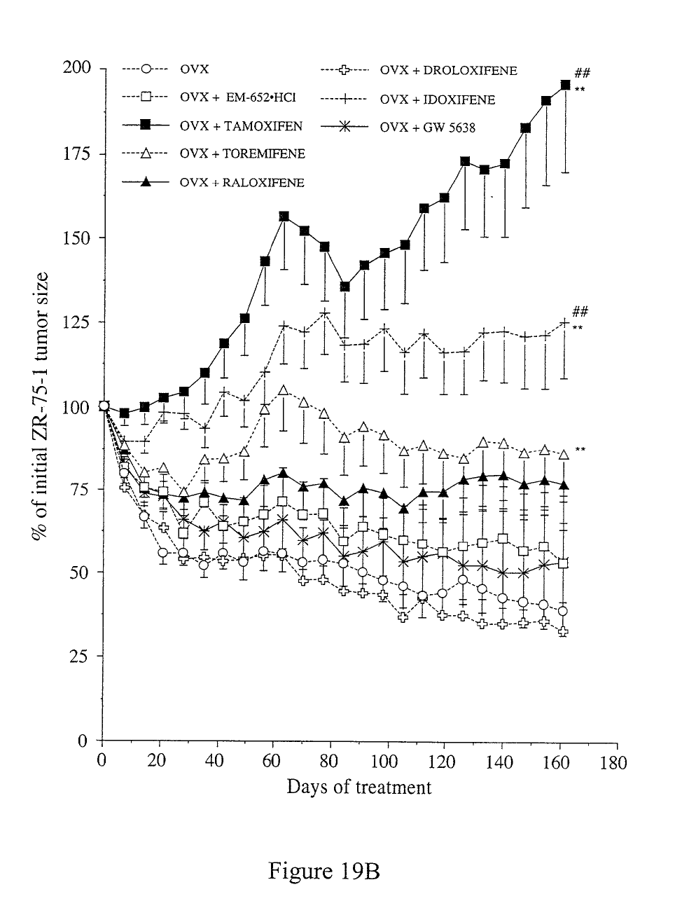

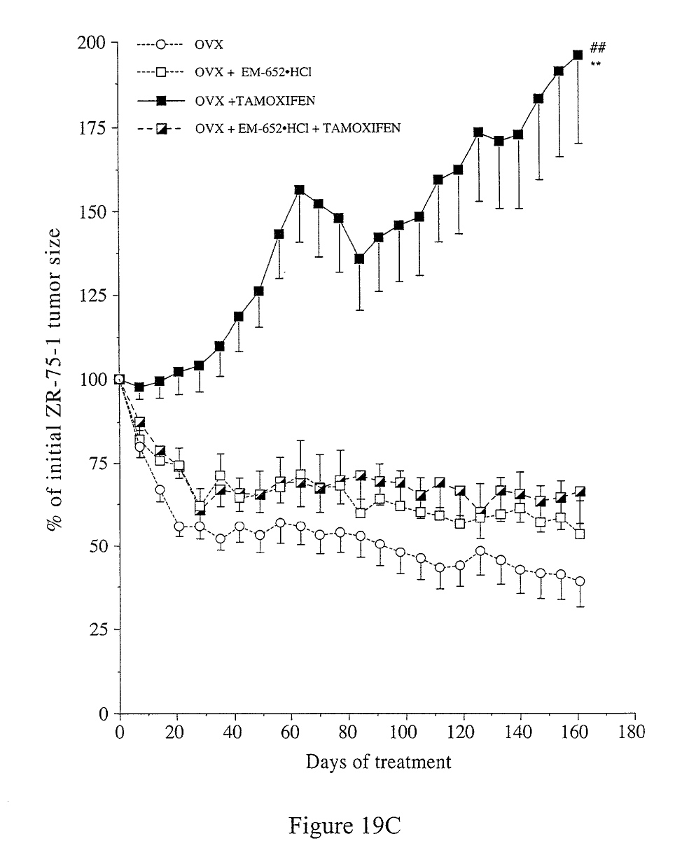

Estrone alone (OVX+E₁) caused a 707% increase in ZR-75-1 tumor size during the 23 week-treatment period (FIG. 19A). Administration of the pure antiestrogen EM-652.HCl at the daily oral dose of 50 μg to estrone-stimulated mice completely prevented tumor growth. In fact, not only tumor growth was prevented but after 23 weeks of treatment, tumor size was 26% lower than the initial value at start of treatment (p<0.04). This value obtained after treatment with EM-652•HCl was not statistically different from that observed after ovariectomy alone (OVX) where tumor size decreased by 61% below initial tumor size. At the same dose (501 μg) and treatment period, the six other antiestrogens did not decrease initial average tumor size. Tumors in these groups were all significantly higher than the OVX control group and to the EM-652•HCl-treated group (p<0,01). In fact, compared to pretreatment values, 23 weeks of treatment with droloxifene, toremifene, GW 5638, raloxifene, tamoxifen and idoxifene led to average tumor sizes 478%, 230%, 227%, 191%, 87% and 86% above pretreatment values, respectively (FIG. 19A).

Agonistic Effects on ZR-75-1 Tumor Growth

After 161 days of treatment with a daily dose of 200 μg of tamoxifen, in the absence of estrone supplementation, the average tumor size increased to 196% over baseline (p<0,01 vs OVX) (FIG.19B). On the other hand, the average tumor size of mice treated with Idoxifene increased (125%) (p<0,01) while tumor size in mice treated with toremifene increased by 86% (p<0,01) (FIG. 19B). The addition of 200 μg of EM-652•HCl to 200 μg of tamoxifen completely inhibited the proliferation observed with tamoxifen alone (FIG. 19C). On the other hand, treatment with EM-652•HCl (p=0,44), raloxifene (p=0,11), droloxifene (p=0,36) or GW 5638 (p=0,17) alone did not significantly change ZR-75-1 tumor size compared to the OVX control group, at the end of the experiment. (FIG. 19B).

Effects on Categories Response

Effects of 50 μg of antiestrogen on estrone stimulation

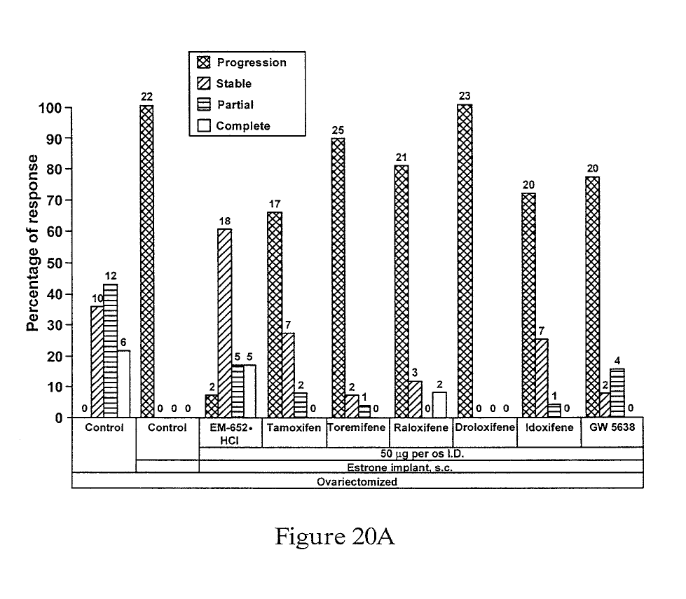

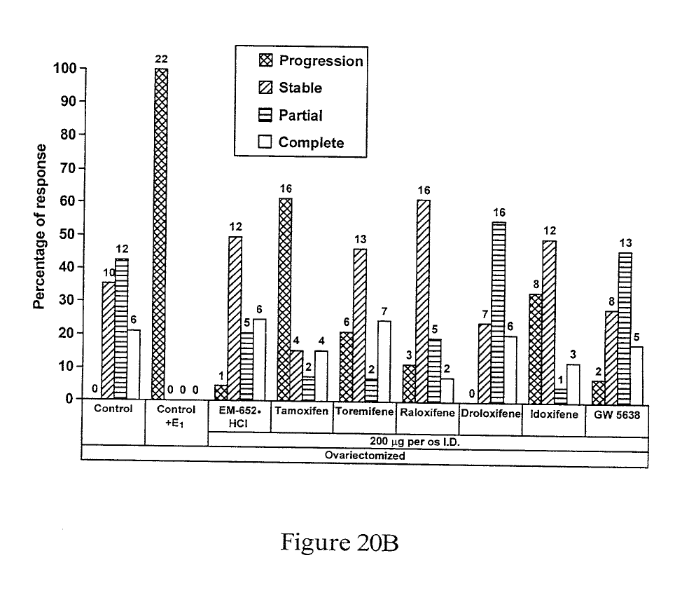

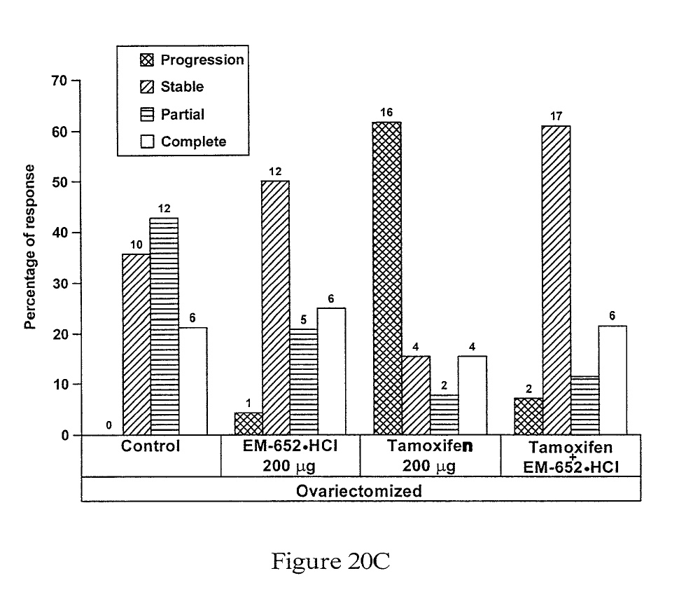

In addition to the effect on tumor size, the category of response achieved by each individual tumor at the end of the experiment is an important parameter of treatment efficacy. In ovariectomized mice, complete, partial, and stable responses were achieved in 21%, 43% and 38% of tumors, respectively, and none of the tumors progressed. On the other hand, in OVX animals supplemented with estrone, 100% of tumors have progressed (FIG. 20A). In the EM-652•HCl-treated group of OVX animals supplemented with estrone, complete, partial, and stable responses were seen in 17%, 17%, and 60% of tumors, respectively and only 7% (2 tumors out of 30) have progressed. Under the same conditions of estrone stimulation, treatment with a daily 50 μg dose of any of the other antiestrogens was unable to decrease the percentage of progressing tumors under 60%. In fact, 65% of tumors (17 of 26) progressed in the tamoxifen-treated group, while 89% (25 of 28) progressed with toremifene, 81% progressed (21 of 26) with raloxifene, 100% (23 of 23) progressed with droloxifene, while 71% (20 of 28) progressed with idoxifene and 77% (20 of 26) progressed with GW 5638 (FIG. 20A).

Effects of 200 μg of antiestrogen in the absence of estrone stimulation on Categories response.

As illustrated in FIG. 20B, tamoxifen, idoxifene and toremifene led to greater proportion of progressing tumors, in the absence of estrone stimulation, than the other antiestrogens. In fact, 62% (16 of 26), 33% (8 of 24) and 21% (6 of 28) of tumors were in the progression category after tamoxifen-, idoxifene- and toremifene treatment at the daily dose of 200 μg, respectively. As can be seen in FIG. 20C, the addition of 200 μg of EM-652•HCl to tamoxifen reduced the percentage of progressing tumors with tamoxifen alone from 62% (16 of 26) to 7% when EM-652.HCl was added to tamoxifen (2 of 28).

Effects of Antiestrogens on Thickness of Uterine Epithelial Cells.

The height of the endometrial epithelial cells was measured as the most direct parameter of agonistic and antagonistic effect of each compound in the endometrium.

Effect of daily 50 μg of antiestrogen in the presence of estrone stimulation on thickness of uterine epithelial cells.

At the daily oral dose of 50 μg, EM-652.HCl inhibited the stimulatory effect of estrone on epithelial height by 70%. The efficacy of the six other antiestrogens tested were significantly lower (p<0,01). In fact, droloxifene, GW 5638, raloxifene, tamoxifen, toremifene and idoxifene inhibited estrone stimulation by 17%, 24%, 26%, 32%, 41% and 50%, respectively. (Table-10).

Effect of daily 200 μg of antiestrogen in absence of estrone stimulation on thinkness of uterine epithelial cells.

In the absence of estrone stimulation, EM-652•HCl and droloxifene were the only compounds tested that did not significantly increase the height of epithelial cells (114% and 101% of the OVX control group value, respectively). Tamoxifen (155%), toremifene (135%) and idoxifene (176%) exerted a significant stimulation of uterine epithelial height (p<0,01 vs OVX control group). Raloxifene (122%) and GW 5638 (121%) also exerted a statistically significant stimulation of uterine epithelial height (p<0,05 vs OVX control group (Table 10). The agonistic and antagonistic effects of each antiestrogen measured on uterine and vaginal weight were in accordance with the pattern observed on uterine epithelium thickness (Data not shown).

TABLE 10

| | | ENDOMETRIAL | |
|---|---|---|---|
| | | EPITHELIUM | |
| | | THICKNESS | |
| GROUP | n | (μm) | ± SEM |
| OVX CONTROL | 14 | 18.31 | ± 0.04 |
| OVX + E₁ CONTROL | 8 | 40.58^{b, d} | ± 0.63 |
| OVX + E₁ + EM-652 · HCl | 14 | 25.06^{b} | ± 0.07 |
| OVX + E₁ + TAMOXIFEN | 10 | 33.44^{b, d} | ± 0.04 |
| OVX + E₁ + TOREMIFENE | 13 | 31.47^{b, d} | ± 0.04 |
| OVX + E₁ + RALOXIFENE | 12 | 34.72^{b, d} | ± 0.06 |
| OVX + E₁ + DROLOXIFENE | 12 | 36.71^{b, d} | ± 0.12 |
| OVX + E₁ + IDOXIFENE | 12 | 29.35^{b, d} | ± 0.05 |
| OVX + E₁ + GW 5638 | 12 | 35.30^{b, d} | ± 0.07 |
| OVX + EM-652 · HCl | 12 | 20.79 | ± 0.10 |
| OVX + TAMOXIFEN | 11 | 28.47^{b, d} | ± 0.05 |
| OVX + EM-652 · HCl + TAMOXIFEN | 13 | 27.95^{b, d} | ± 0.06 |
| OVX + TOREMIFENE | 13 | 24.75^{b, c} | ± 0.04 |
| OVX + RALOXIFENE | 12 | 22.33^{a} | ± 0.05 |
| OVX + DROLOXIFENE | 13 | 18.50 | ± 0.07 |
| OVX + IDOXIFENE | 11 | 32.14^{b, d} | ± 0.05 |
| OVX + GW 5638 | 13 | 22.22^{a} | ± 0.05 |

^{a, b}Experimental versus OVX control mice: ^{a}P < 0.05; ^{b}p < 0.01.
^{c, d}Experimental versus EM-652 · HCl treated-mice: ^{c}P < 0.05; ^{d}P < 0.01.

### Example 9

Radioactivity in the Brain of Female Rats Following a Single Oral Dose of ¹⁴C-EM-800 (20 mg/kg)

Example 9 shows the radioactivity in brain of rats following single oral dose of ¹⁴C-EM-800 (20 mg/kg). For comparison purposes, values for the blood, plasma, liver and uterus from each of these animals were included. These results are from LREM study No. 1129 Tissue Distribution and Excretion of Radioactivity Following a Single Oral Dose of ¹⁴C-EM-800 (20 mg/2 ml/kg) to Male and Female Long-Evans Rats. These numbers indicate that the amount of total drug-derived radioactivity in the brain of female Long-Evans rats was very low (ng equiv/g tissue) and was not detected after 12 hr post dose. At 2 hours, radioactivity in the brain was 412 lower than in liver, 21 times lower than in the uterus, 8.4 times lower that in the blood and 13 times lower than in plasma. Since an unknown proportion of total brain radioactivity is due to contamination by blood radioactivity, the values shown in Table 1 for brain radioactivity are an overestimate of the level of ¹⁴C (EM-800)-related radioactivity in the brain tissue itself. Such data suggest that the level of the antiestrogen in the brain tissue is too low, if existant, to counteract the effect of exogenous estrogen. It is important to note that some of the radioactivity detected in the brain tissue may be due to residual blood in the tissue. Additionally, the radiochemical purity of the ¹⁴C-EM-800 used for this study was minimally 96.25%.

TABLE 11
Mean Concentration of Drug-Derived Radioactivity (ng EM-800 equiv/g
tissue) in Selected Tissues of Female Long-Evans Rats Following a
Single Oral Dose of ¹⁴C-EM-800 (20 mg/kg)^{a}

| Time | Brain | | Blood | | Plasma | |
|---|---|---|---|---|---|---|
| (hr) | Mean^{b} | (% CV) | Mean^{b} | (% CV) | Mean^{b} | (% CV) |
| 2 | 17.6 | (29) | 148.7 | (22) | 224.6 | (20) |
| 4 | 17.1 | (29) | 66.9 | (45) | 103.2 | (39) |
| 6 | 15.6 | (8) | 48.3 | (29) | 74.1 | (31) |
| 8 | 16.8 | (31) | 41.1 | (12) | 64.1 | (14) |
| 12 | 10.0^{c} | (87) | 28.7 | (54) | 40.7 | (55) |
| 24 | 0 | (NC) | 4.7^{d} | (173) | 10.1 | (86) |
| 36 | 0 | (NC) | 0 | (NC) | 0 | (NC) |
| 48 | 0 | (NC) | 0 | (NC) | 0 | (NC) |
| 72 | 0 | (NC) | 0 | (NC) | 0 | (NC) |
| 96 | 0 | (NC) | 0 | (NC) | 0 | (NC) |
| 168 | 0 | (NC) | 0 | (NC) | 0 | (NC) |

^{a}Values from report tables for LREM 1129 (EM-800: Tissue Distribution and Excretion of Radioactivity Following a Single Oral Dose of ¹⁴C-EM-800 (20 mg/2 mL/kg) to Male and Female Long-Evans Rats).
^{b}Limit of quantification (LOQ) of 1.2 ng EM-800 equivalent.
^{c}One sample below the LOQ; 0 used in calculation of mean.
^{d}Two samples below the LOQ; 0 used in calculation of mean.
% CV: Coefficient of variation expressed as a percent, where n = 3.
NC: Not calculated.

TABLE 12
Mean Concentration of Drug-Derived Radioactivity (μg EM-800 equiv/g tissue) in Selected Tissues
of Female Long-Evans Rats Following a Single Oral Dose of ¹⁴C-EM-800 (20 mg/kg)^{a}

| Time | Brain | | Liver | | Uterus | | Blood | | Plasma | |
|---|---|---|---|---|---|---|---|---|---|---|
| (hr) | Mean^{b} | (% CV) | Mean^{b} | (% CV) | Mean^{b} | (% CV) | Mean^{b} | (% CV) | Mean^{b} | (% CV) |
| 2 | 0.0176 | (29) | 7.2547 | (30) | 0.3675 | (36) | 0.1487 | (22) | 0.2246 | (20) |
| 4 | 0.0171 | (29) | 3.2201 | (48) | 0.2866 | (83) | 0.0669 | (45) | 0.1032 | (39) |
| 6 | 0.0156 | (8) | 2.7462 | (8) | 0.2757 | (19) | 0.0483 | (29) | 0.0741 | (31) |
| 8 | 0.0168 | (31) | 2.7748 | (8) | 0.3332 | (46) | 0.0411 | (12) | 0.0641 | (14) |
| 12 | 0.0100^{c} | (87) | 1.8232 | (38) | 0.2407 | (25) | 0.0287 | (54) | 0.0407 | (55) |
| 24 | 0 | (NC) | 0.6391 | (52) | 0.0837 | (54) | 0.0047^{d} | (173) | 0.0101 | (86) |
| 36 | 0 | (NC) | 0.4034 | (22) | 0.0261 | (15) | 0 | (NC) | 0 | (NC) |
| 48 | 0 | (NC) | 0.2196 | (37) | 0.0238 | (44) | 0 | (NC) | 0 | (NC) |
| 72 | 0 | (NC) | 0.1326 | (4) | 0 | (NC) | 0 | (NC) | 0 | (NC) |
| 96 | 0 | (NC) | 0.0944 | (15) | 0 | (NC) | 0 | (NC) | 0 | (NC) |
| 168 | 0 | (NC) | 0.0348 | (14) | 0 | (NC) | 0 | (NC) | 0 | (NC) |

^{a}Values from report tables for LREM 1129 (EM-800: Tissue Distribution and Excretion of Radioactivity Following a Single Dose of ¹⁴C-EM-800 (20 mg/2 mL/kg) to Male and Female Long-Evans Rats).
^{b}Limit of quantification (LOQ) of 1.2 ng EM-800 equivalent.
^{c}One sample below the LOQ; 0 used in calculation of mean.
^{d}Two samples below the LOQ; 0 used in calculation of mean.
% CV: Coefficient of variation expressed as a percent, where n = 3.
NC: Not calculated.

### Example 10

### Combination of the Antiestrogen EM-652.HCl with Estradiol Protects Against Uterine Stimulation

Materials and Methods

Animals and Treatment

Ten to twelve week-old female Sprague-Dawley rats (Crl:CD(SD)Br) (Charles River Laboratory, St-Constant, Canada) weighing 215-265g at time of ovariectomy were used. The animals were housed individually in an environmentally-controlled room (temperature: 22±3° C.; humidity: 50±20%; 12-h light-12-h dark cycles, lights on at 07:15 h). The animals were allowed free access to tap water and a certified rodent feed (Lab Diet 5002 (pellet), Ralston Purina, St-Louis, Mo.). The experiment was conducted in an animal facility approved by the Canadian Council on Animal Care (CCAC) and the Association for Assessment and Accreditation of Laboratory Animal Care (AAALAC) in accordance with the CCAC Guide for Care and Use of Experimental Animals.

One hundred thirty-seven rats were randomly distributed between 10 groups of 13 or 14 animals each as follows: 1) Intact control; 2) Ovariectomized (OVX) control; 3) OVX+17β-estradiol (E₂; 2 mg/kg); groups 4 to 10) OVX+E₂+EM-652.HCl (0.01, 0.03, 0.1, 0.3, 1, 3 or 10 mg/kg). On the first day of the study, the animals of the appropriate groups were bilaterally ovariectomized (OVX) under isoflurane anesthesia. The tested compounds were then given once daily by oral gavage as a suspension in 0.4% methylcellulose (0.5 ml/rat) from day 1 to day 14 of the study. Animals of groups 1 and 2 received the vehicle alone during the same time period. On day 15 of the study, 4 animals per group were perfused with 10% buffered formalin and tissues were processed for histological examination. The other animals were killed by exsanguination at the abdominal aorta under isoflurane anesthesia.

The uterus and vagina were removed, stripped of remaining fat and weighed. A specimen from each uterus was fixed in 10% buffered formalin for determination of the height of endometrial epithelial cells using a computerized-assisted program (Software Image-Pro Plus).

Serum Cholesterol Levels

Total cholesterol was measured on serum samples collected from overnight fasted animals using a Boehringer Mannheim Diagnostic Hitachi 911 Analyzer (Boehringer Mannheim Diagnostic Laboratory Systems).

Statistical Analyses

Data are expressed as the means±SEM. Statistical significance was determined according to the multiple-range test of Duncan-Kramer (Kramer, Biometrics, 12: 307-310, 1956).

Results

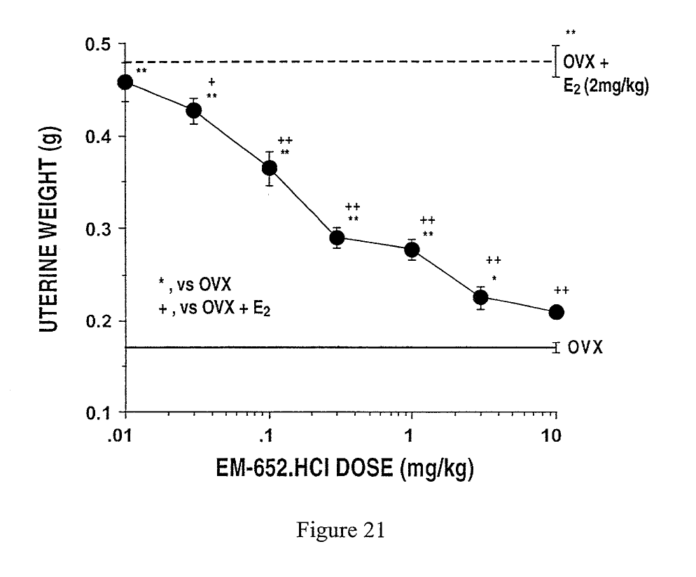

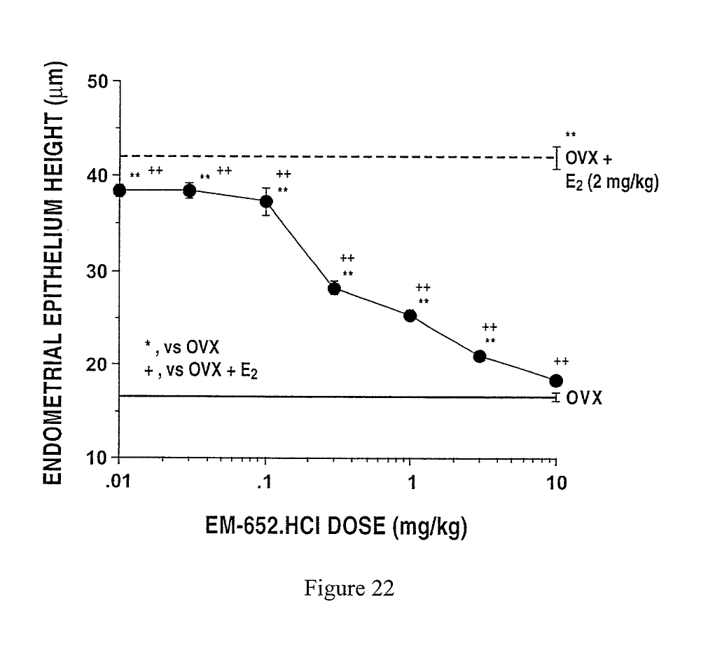

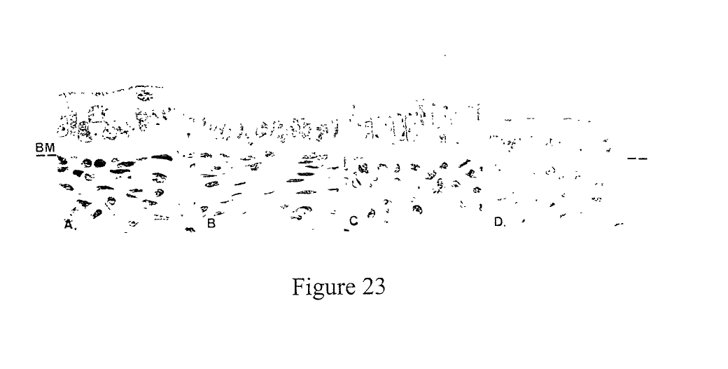

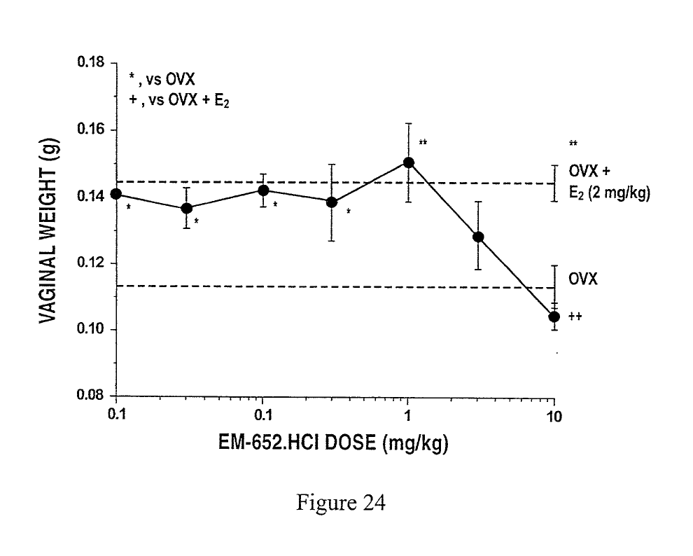

The 65% reduction in uterine weight observed two weeks after ovariectomy was completely reversed by daily oral administration of 17β-estradiol (E₂) at the 2 mg/kg dose (490±26 mg versus 480±17 mg; N.S.) (FIG. 21). As illustrated in the same Figure, a progressive inhibition of the stimulatory effect of E₂ on uterine weight was observed with increasing doses of EM-652.HCl, 84% and 87% reversals of the effect of E₂ being observed at the 3 mg/kg and 10 mg/kg doses of the antiestrogen, respectively. Comparable results were obtained on endometrial epithelial height (FIG. 22). In fact, E₂-stimulated endometrial epithelial height was 83% and 93% prevented by the 3 mg/kg and 10 mg/kg doses of the antiestrogen, respectively. Endometrial epithelial height was higher (34.7%, p<0.01) in the group of OVX animals treated with E₂ (41.9±1.2 μm) compound compare to intact control animals (31.1±0.7 μm) (FIG. 23). E₂ supplementation of OVX animals led to vaginal weight similar to that of intact animals (149.9±9.0 mg versus 145±5.3 mg, N.S.). It can be seen in FIG. 24 that the inhibition of vaginal weight was observed at higher doses of EM-652.HCl than observed on uterine weight. In fact, no significant inhibitory effect of the antiestrogen on vaginal weight was observed up to 1 mg/kg of the compound. In fact, while the 3 mg/kg dose of EM-652.HCl caused a statistically non significant 50% inhibition of the stimulatory effect of E₂, a complete reversal of the effect of E₂ on vaginal weight was observed at the 10 mg/kg dose of the antiestrogen.

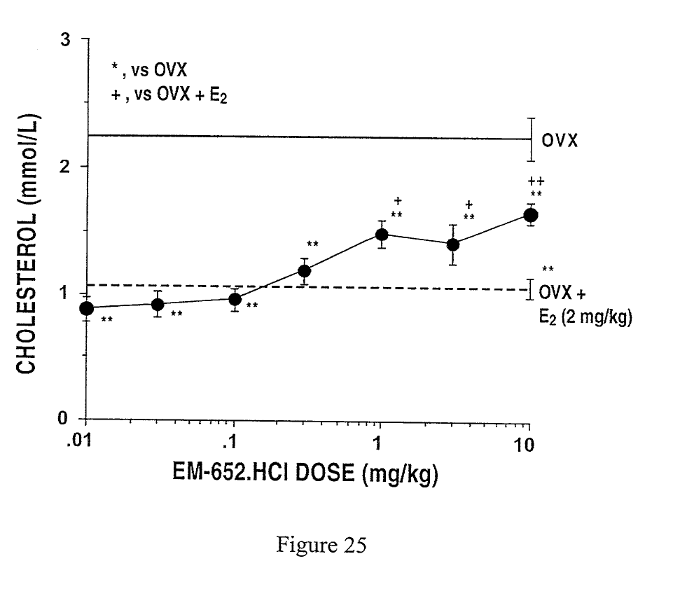

A 37% increase in serum cholesterol was observed 2 weeks after OVX (p<0.01). Treatment of OVX animals with E₂, on the other hand, caused a 53% (p<0.01) inhibition of serum cholesterol levels (FIG. 25). The addition of EM-652.HCl at the daily doses of 0.01 mg/kg to 0.3 mg/kg had no statistically significant effect on the inhibitory action of E₂. On the other hand, the 1.0, 3.0 and 10 mg/kg doses of EM-652.HCl reduced by 36%, 30% and 50%, respectively, the effect of E₂. The present data clearly demonstrate that the antiestrogen, EM-652.HCl neutralises the stimulatory effect of E₂ on uterine weight and endometrial epithelial height, two well recognized parameters of estrogen action in peripheral tissues. Such data clearly suggest that the co-administration of EM-652.HCl in postmenopausal women receiving estradiol for the relief of vasomotor symptoms will prevent the stimulatory effect of estrogens on the endometrium.

The 36%, 30% and 50% reversals of the inhibitory effect of the 1.0 mg/kg, 3.0 mg/kg and 10 mg/kg doses of EM-652.HCl can probably be explained by the predominance of the effect of the antiestrogen which would probably have led to the same degree of inhibition if used alone. In fact, EM-652.HCl has an affinity for the rat uterine ER approximately 5-fold higher than E₂ itself (Martel et al., J. Steroid Biochem. Molec. Biol., 64: 199-205, 1998).

### Pharmaceutical Composition and Kit Examples

Set forth below, by way of example and not of limitation, are several pharmaceutical composition and kits utilizing preferred active SERM EM-800 or EM-652.HCl (EM-1538) and preferred active estrogen 17β-estradiol, ethinylestradiol or conjugated estrogens. Other compounds of the invention or combination thereof, may be used in place of (or in addition to) EM-800 or EM-652.HCl or 17β-estradiol or ethinylestradiol. The concentration of active ingredient may be varied over a wide range as discussed herein. The amounts and types of other ingredients that may be included are well known in the art.

### Example A

Pharmaceutical composition for orally administration (capsules)

| | Weight % |
|---|---|
| Ingredient | (by weight of total composition) |
| EM-652.HCl | 5.0 |
| Ethinylestradiol | 0.02 |
| Lactose hydrous | 79.98 |
| Starch | 4.8 |
| Cellulose microcrystalline | 9.8 |
| Magnesium stearate | 0.4 |

or

| | Weight % |
|---|---|
| Ingredient | (by weight of total composition) |
| EM-652.HCl | 5.0 |
| Conjugated estrogens | 0.2 |
| Lactose hydrous | 79.8 |
| Starch | 4.8 |
| Cellulose microcrystalline | 9.8 |
| Magnesium stearate | 0.4 |

### Example B

### Kit

The SERM and estrogen are orally administered

| | Weight % |
|---|---|
| Ingredient | (by weight of total composition) |
| EM-652.HCl | 5.0 |
| Lactose hydrous | 80.0 |
| Starch | 4.8 |
| Cellulose microcrystalline | 9.8 |
| Magnesium stearate | 0.4 |

### Estrogen Composition for Oral Administration (Gelatin Capsule)

| | Weight % |
|---|---|
| Ingredient | (by weight of total composition) |
| Ethinylestradiol | 0.02 |
| Lactose hydrous | 84.98 |
| Starch | 4.8 |
| Cellulose microcrystalline | 9.8 |
| Magnesium stearate | 0.4 |

Other SERMs may be substituted for EM-800 or EM-01538 in the above formulations, as well as other estrogens may be substituted for 17β-estradiol, ethinylestradiol or conjugated estrogens. More than one SERM or more than one estrogen may be included in which case the combined weight percentage is preferably that of the weight percentage for the single estrogen or single SERM given in the examples above.

The invention has been described in terms of preferred embodiments and examples, but is not limited thereby. Those of skill in the art will readily recognize the broader applicability and scope of the invention which is limited only by the patent claims herein.

What is claimed is:

1. A pharmaceutical composition comprising:
a) a pharmaceutically acceptable excipient, diluent or carrier;
b) a therapeutically effective amount of at least one estrogen or prodrug thereof; and
c) a therapeutically effective amount of at least one selective estrogen receptor modulator or prodrug thereof, wherein said modulator is a different compound from said estrogen and said modulator is not a benzothiophene derivative, a phenylindole derivative, a naphthalene derivative, an isoquinoline derivative or an enantiomeric mixture of 3-phenylquinoline derivatives, 3-phenylthiochroman derivatives, and 3-phenylchroman derivatives having more than 10% of the enantiomer of 2R configuration.

2. A pharmaceutical composition comprising:
a) a pharmaceutically acceptable excipient, diluent or carrier;
b) a therapeutically effective amount of at least one estrogen or prodrug thereof;
(c) a therapeutically effective amount of at least one selective estrogen receptor modulator or prodrug thereof, wherein said modulator is a different compound from said estrogen; and
(d) a therapeutically effective amount of at least one additional agent selected from the group consisting of bisphosphonate, progestogen, an androgenic agent, testosterone, dehydroepiandrosterone, dehydroepiandrosterone-sulfate, androst-5-ene-3β,17β-diol, 4-androstene-3,17-dione, and a prodrug of any of the foregoing additional agents.

3. The pharmaceutical composition of claim 1, wherein the selective estrogen receptor modulator has a molecular formula with the following features:
a) two aromatic rings spaced by 1 to 2 intervening carbon atoms, both aromatic rings being either unsubstituted or substituted by a hydroxyl group or a group converted in vivo to hydroxyl;
b) a side chain possessing an aromatic ring and a tertiary amine function or salt thereof;
and wherein said modulator is not a benzothiophene derivative, a phenylindole derivative, a naphtalene derivative, an isoquinoline derivative or an enantiomeric mixture of 3-phenylquinoline derivatives, 3-pheynulthiochroman derivatives, and 3-phenylchroman derivatives having more than 10% of the enantiomer of 2R configuration.

4. The pharmaceutical composition of claim 3, wherein the side chain is selected from the group consisting of:

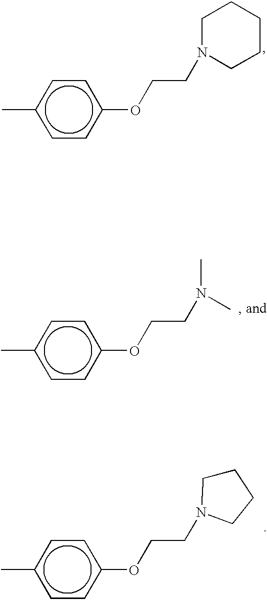

5. The pharmaceutical composition of claim 3, wherein the selective estrogen receptor modulator is selected from the group consisting of a triphenylethylene derivative, benzopyran derivative, HMR 3339, HMR 3656, LY 335124, LY 326315, SH 646, ERA 923 and centchroman derivative.

6. The pharmaceutical composition of claim 2, wherein the selective estrogen receptor modulator is a benzothiophene derivative compound of the following formula: 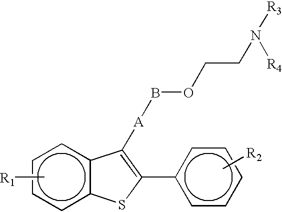
wherein R₁ and R₂ are independently selected from the group consisting of: hydrogen, hydroxyl, and a moiety converted in vivo in hydroxyl;
wherein R₃ and R₄ are either (a) independently C1-C4 alkyl, or (b) a moiety which in combination with the nitrogen to which they are bound, is selected from the group consisting of pyrrolidino, dimethyl-1-pyrrolidino, methyl-1-pyirolidinyl, piperidino, hexamethyleneimino and morpholino;
wherein A is selected from the group consisting of —CO—, —CHOH, and —CH₂—,
wherein B is selected from the group consisting of phenylene, pyridylidene, and —cycloC₄H₂N₂—.

7. The pharmaceutical composition of claim 6, wherein the selective estrogen receptor modulator is selected from the group consisting of Raloxifene, LY 353381 and LY 335563.

8. The pharmaceutical composition of claim 3, wherein the selective estrogen receptor modulator is a triphenylethylene or diphenylhydronaphthalene derivative compound of the following formula: 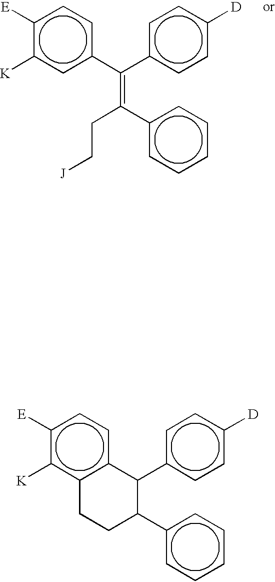
wherein D is —OCH₂CH₂N(R₃)R₄, —OCH₂CH₂OH, or —CH═CH—COOH (R₃ and R₄ either being independently selected from the group consisting of C1-C4 alkyl, or R₃, R₄, and the nitrogen atom to which they are bound, together being a ring structure selected from the group consisting of pyrrolidino, dimethyl-1-pyrrolidino, methyl-1-pyrrolidinyl, piperidino, hexamethyleneimino and morpholino);
wherein E and K are independently hydrogen or hydroxyl, phosphate ester, or lower alkyl, wherein J is hydrogen or halogen.

9. The pharmaceutical composition of claim 3, wherein selective estrogen receptor modulator is OH-tamoxifen, Droloxifene, Toremifene, lodoxifene, Lasofoxifene, iproxifene, FC 1271, and GW5638.

10. The pharmaceutical composition of claim 3, wherein the selective estrogen receptor modulator is a centchroman derivative compound of the following formula: 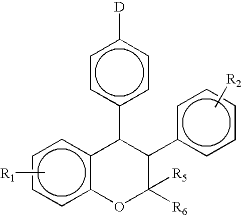
wherein R₁ and R₂ are independently selected from the group consisting of: hydrogen, hydroxyl, and a moiety converted in vivo in hydroxyl;
wherein R₅ and R₆ are independently hydrogen or C₁-C₆ alkyl;
wherein D is —OCH₂CH₂N(R₃)R₄ ( R₃ and R₄ either being independently selected from the group consisting of C₁-C₄ alkyl, or R₃, R₄ and the nitrogen atom to which they are bound, together being a ring structure selected from the group consisting of pyrrolidino, dimethyl-1-pyrrolidino, methyl-1-pyrrolidinyl, piperidino, hexamethyleneimino, morpholino).

11. The pharmaceutical composition of claim 10, wherein the centchroman derivative is (3,4-trans-2,2-dimethyl-3-phenyl-4-[4-(2-(2-(pyrrolidin-1-yl)ethoxy)phenyl]-7-methoxychroman).

12. The pharmaceutical composition of claim 3, wherein the selective estrogen receptor modulator has the following formula: 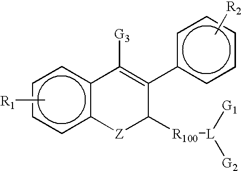
wherein R₁ and R₂ are independently hydrogen, hydroxyl or a moiety which is converted to hydroxyl in vivo;
wherein Z is either absent or selected from the group consisting of —CH₂—, —O—,—S— and —NR₃— (R₃ being hydrogen or lower alkyl);
wherein theR100 is a bivalent moiety which distances L from the B-ring by 4-10 intervening atoms;
wherein L is a bivalent or trivalent moiety selected from the group of —SO—, —CON—, —N<, and —SON<;
wherein G₁ is selected from the group consisting of hydrogen, a C₁ to C₅ hydrocarbon, a bivalent moiety which in combination with G₂ and L is a 5- to 7-membered heterocyclic ring, and halo or unsaturated derivatives of the foregoing;
wherein G₂ is either absent or selected from the group consisting of hydrogen, a C₁ to C₅ hydrocarbon, a bivalent moiety which in combination with G₁ and L is a 5- to 7-membered heterocyclic ring, and halo or unsaturated derivatives of the foregoing;
wherein G₃ is selected from the group consisting of hydrogen, methyl and ethyl.

13. The pharmaceutical composition of claim 12, wherein the compound is a benzopyran derivative of the following general structure: 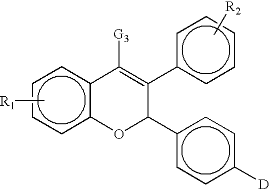
or a pharmaceutically acceptable salt thereof,
wherein D is —OCH₂CH₂N(R₃)R₄ (R₃ and R₄ either being independently selected from the group consisting of C₁-C₄ alkyl, or R₃, R₄ and the nitrogen atom to which they are bound, together being a ring structure selected from the group consisting of pyrrolidino, dimethyl-1-pyrrolidino, methyl-1-pyrrolidinyl, piperidino, hexamethyleneimino, morpholino);
wherein R₁ and R₂ are independently selected from the group consisting of: hydrogen, hydroxyl, and a moiety converted in vivo in hydroxyl.

14. The pharmaceutical composition of claim 13, wherein the benzopyran derivative is optically active due to a majority of its stereoisomer having an absolute configuration S on carbon 2, said compound having the molecular structure: 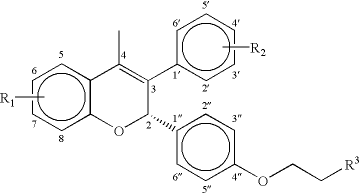
wherein R₁ and R₂ are independently selected from the group consisting of hydroxyl and a moiety convertible in vivo to hydroxyl;
wherein R³ is a species selected from the group consisting of saturated, unsaturated or substituted pyrrolidinyl, saturated, unsaturated or substituted piperidino, saturated, unsaturated or substituted piperidinyl, saturated, unsaturated or substituted morpholino, nitrogen-containing cyclic moiety, nitrogen-containing polycyclic moiety, and NRaRb (Ra and Rb being independently hydrogen, straight or branched C₁-C₆ alkyl, straight or branched C₂-C₆ alkenyl, and straight or branched C₂-C₆ alkynyl).

15. The pharmaceutical composition of claim 14, wherein said compound or salt substantially lacks (2R)-enantiomer.

16. The pharmaceutical composition of claim 14, wherein said selective estrogen receptor modulator is selected from the group consisting of: 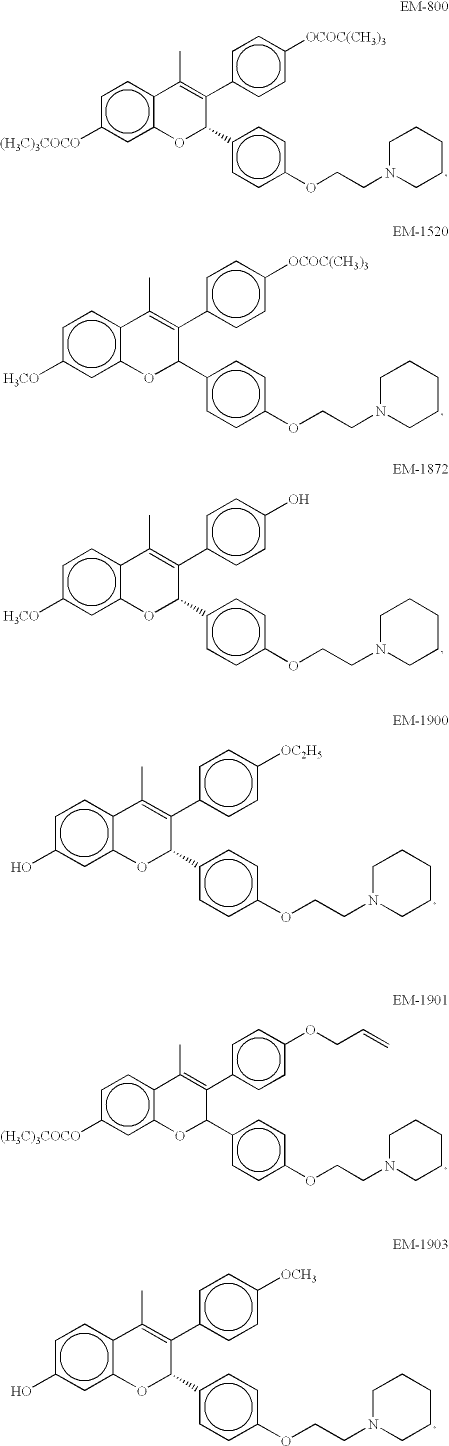
wherein all of the foregoing molecular structures whose stereochemistry is indicated are optically active due to a majority of their stereoisomers being of 2S configuration.

17. The pharmaceutical composition of claim 14, wherein the benzopyran derivative is a salt of an acid selected from the group consisting of acetic acid, adipic acid, benzenesulfonic acid, benzoic acid, camphorsulfonic acid, citric acid, fumaric acid, hydroiodic acid, hydrobromic acid, hydrochloric acid, hydrochlorothiazide acid, hydroxy-naphthoic acid, lactic acid, maleic acid, methanesulfonic acid, methylsulfuric acid, 1,5-naphthalenedisulfonic acid, nitric acid, palmitic acid, pivalic acid, phosphoric acid, propionic acid, succinic acid, sulfuric acid, tartaric acid, terephthalic acid, p-toluenesulfonic acid, and valeric acid.

18. The pharmaceutical composition of claim 17, wherein the acid is hydrochloric acid.

19. The pharmaceutical composition of claim 1, wherein said selective estrogen receptor modulator is: 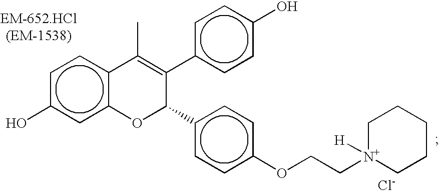
and is optically active due to a majority of its stereoisomers being of 2S configuration; and
wherein the estrogen is selected from the group consisting of 17β-estradiol, 17β-estradiol esters, 17α-estradiol, 17α-estradiol esters, estriol, estriol esters, estrone, estrone esters, conjugated estrogen, equilin, equilin esters, 17α-ethynylestradiol, 17α-ethynylestradiol esters, mestranol, and mestranol esters.

20. The pharmaceutical composition of claim 1, wherein said estrogen is selected from the group consisting of 17β-estradiol, 17β-estradiol esters, estriol, estriol esters, estrone, estrone esters, conjugated estrogen, equilin, equilin esters, 17α-ethynylestradiol, 17α-ethynylestradiol esters, mestranol, mestranol esters, chemestrogen, DES, phytestrogen, tibolone, 2′-ethylestrogenoxazole, and ethynediol.

21. The pharmaceutical composition of claims 1, wherein said estrogen is a mixed estrogenic/androgenic compound.

22. A kit comprising a first container containing a pharmaceutical formulation comprising a therapeutically effective amount of at least one estrogen or a prodrug thereof; and said kit further comprising a second container containing a pharmaceutical formulation comprising a therapeutically effective amount of at least one selective estrogen receptor modulator or prodrug thereof, said modulator not being a benzothiophene or a phenylindole derivative.

23. A kit comprising a first container containing a pharmaceutical formulation comprising a therapeutically effective amount of at least one estrogen or a prodrug thereof, and said kit further comprising a second container containing a pharmaceutical formulation comprising a therapeutically effective amount of at least one selective estrogen receptor modulator or prodrug thereof, comprising at least one additional container of said kit that contains a therapeutically effective amount of at least one additional agent selected from the group consisting of dehydroepiandrosterone, dehydroepiandrosterone-sulfate, androst-5-ene-3β,17β-diol, an androgenic agent, testosterone, 4-androstene-3,17-dione and a prodrug of any of the foregoing additional agents.

24. The kit of claim 22 further comprising at least one additional container containing a pharmaceutical formulation comprising a therapeutically effective amount of at least one bisphosphonate.

25. The kit of claims 22, wherein the selective estrogen receptor modulator has a molecular formula with the following features:
a) two aromatic rings spaced by 1 to 2 intervening carbon atoms, both aromatic rings being either unsubstituted or substituted by a hydroxyl group or a group converted in vivo to hydroxyl;
b) a side chain possessing an aromatic ring and a tertiary amine function or salt thereof;
and wherein said modulator is not a benzothiophene derivative, a phenylindole derivative, a naphtalene derivative, an isoquinoline derivative or an enantiomeric mixture of 3-phenylquinoline derivatives, 3-pheynulthiochroman derivatives, 3-phenylchroman derivatives having more than 10% of the enantiomer of 2R configuration.

26. The kit of claim 25, wherein the side chain is selected from the group consisting of:

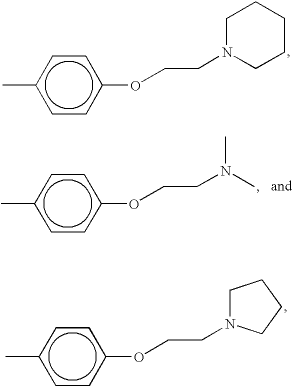

27. The kit of claim 25, wherein the selective estrogen receptor modulator is selected from the group consisting of a triphenylethylene derivative, benzopyran derivative, HMR 3339, HMR 3656, LY 335124, LY 326315, SH 646, ERA 923 and centchroman derivative.

28. The kit of claim 23, wherein the selective estrogen receptor modulator is a benzothiophene derivative compound of the following formula: 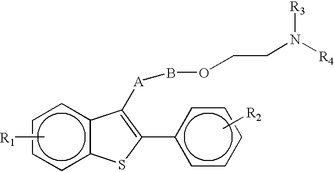
wherein R₁ and R₂ are independently selected from the group consisting of: hydrogen, hydroxyl, and a moiety converted in vivo in hydroxyl;
wherein R₃ and R₄ are either (a) independently C1-C4 alkyl, or (b) a moiety which in combination with the nitrogen to which they are bound, is selected from the group consisting of pyrrolidino, dimethyl-1-pyrrolidino, methyl-1-pyrrolidinyl, piperidino, hexamethyleneimino and morpholino;
wherein A is selected from the group consisting of —CO—, —CHOH, and —CH₂—;
wherein B is selected from the group consisting of phenylene, pyridylidene, and —cycloC₄H₂N₂—.

29. The kit of claim 28, wherein the selective estrogen receptor modulator is selected from the group consisting of Raloxifene, LY 353381 and LY 335563.

30. The kit of claim 25, wherein the selective estrogen receptor modulator is a triphenylethylene or diphenylhydronaphthalene derivative compound of the following formula: 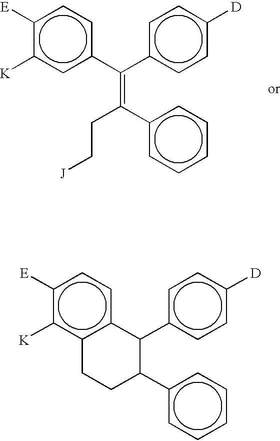
wherein D is —OCH₂CH₂N(R₃)R₄, —OCH₂CH₂OH, or —CH═CH—COOH (R₃ and R₄ either being independently selected from the group consisting of C1-C4 alkyl, or R₃, R₄, and the nitrogen atom to which they are bound, together being a ring structure selected from the group consisting of pyrrolidino, dimethyl-1-pyrrolidino, methyl-1-pyrrolidinyl, piperidino, hexamethyleneimino and morpholino);
wherein E and K are independently hydrogen or hydroxyl, phosphate ester, or lower alkyl, wherein J is hydrogen or halogen.

31. The kit of claim 22, wherein selective estrogen receptor modulator is OH-tamoxifen, Droloxifene, Toremifene, Iodoxifene, Lasofoxifene, iproxifene, FC 1271, and GW5638.

32. The kit of claim 25, wherein the selective estrogen receptor modulator is a centchroman derivative compound of the following formula: 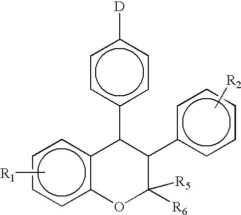
wherein R₁ and R₂ are independently selected from the group consisting of: hydrogen, hydroxyl, and a moiety converted in vivo in hydroxyl;
wherein R₅ and R₆ are independently hydrogen or C₁-C₆ alkyl;
wherein D is —OCH₂CH₂N(R₃)R₄ (R₃ and R₄ either being independently selected from the group consisting of C₁-C₄ alkyl, or R₃, R₄ and the nitrogen atom to which they are bound, together being a ring structure selected from the group consisting of pyrrolidino, dimethyl-1-pyrrolidino, methyl-1-pyrrolidinyl, piperidino, hexamethyleneimino, morpholino).

33. The kit of claim 32, wherein the centchroman derivative is (3,4-trans-2,2-dimethyl-3-phenyl-4-[4-(2-(2-(pyrrolidin-1-yl)ethoxy)phenyl]-7-methoxychroman).

34. The kit of claim 25, wherein the selective estrogen receptor modulator has the following formula: 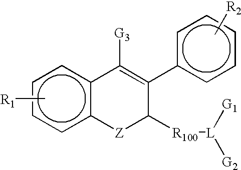
wherein R₁ and R₂ are independently hydrogen, hydroxyl or a moiety which is converted to hydroxyl in vivo;
wherein Z is either absent or selected from the group consisting of —CH₂—,—O—,—S— and —NR₃— (R₃ being hydrogen or lower alkyl);
wherein theR100 is a bivalent moiety which distances L from the B-ring by 4-10 intervening atoms;
wherein L is a bivalent or trivalent moiety selected from the group of —SO—, —CON—, —N<, and —SON<;
wherein G₁ is selected from the group consisting of hydrogen, a C₁ to C₅ hydrocarbon, a bivalent moiety which in combination with G₂ and L is a 5- to 7-membered heterocyclic ring, and halo or unsaturated derivatives of the foregoing;
wherein G₂ is either absent or selected from the group consisting of hydrogen, a C₁ to C₅ hydrocarbon, a bivalent moiety which in combination with G₁ and L is a 5- to 7-membered heterocyclic ring, and halo or unsaturated derivatives of the foregoing;
wherein G₃ is selected from the group consisting of hydrogen, methyl and ethyl.

35. The kit of claim 34, wherein the compound is a benzopyran derivative of the following general structure: 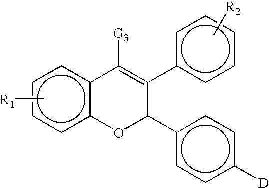
or a pharmaceutically acceptable salt thereof,
wherein D is —OCH₂CH₂N(R₃)R₄ (R₃ and R₄ either being independently selected from the group consisting of C₁-C₄ alkyl, or R₃, R₄ and the nitrogen atom to which they are bound, together being a ring structure selected from the group consisting of pyrrolidino, dimethyl-1-pyrrolidino, methyl-1-pyrrolidinyl, piperidino, hexamethyleneimino and morpholino);
wherein R₁ and R₂ are independently selected from the group consisting of: hydrogen, hydroxyl, and a moiety converted in vivo in hydroxyl.

36. The kit of claim 35, wherein the benzopyran derivative is optically active due to a majority of its stereoisomer having an absolute configuration S on carbon 2, said compound having the molecular structure: 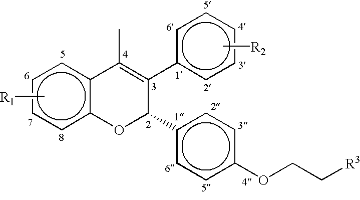
wherein R₁ and R₂ are independently selected from the group consisting of hydroxyl and a moiety convertible in vivo to hydroxyl;
wherein R³ is a species selected from the group consisting of saturated, unsaturated or substituted pyrrolidinyl, saturated, unsaturated or substituted piperidino, saturated, unsaturated or substituted piperidinyl, saturated, unsaturated or substituted morpholino, nitrogen-containing cyclic moiety, nitrogen-containing polycyclic moiety, and NRaRb (Ra and Rb being independently hydrogen, straight or branched C₁-C₆ alkyl, straight or branched C₂-C₆ alkenyl, and straight or branched C₂-C₆ alkynyl).

37. The kit of claim 36, wherein said compound or salt substantially lacks (2R)-enantiomer.

38. The kit of claim 36, wherein said selective estrogen receptor modulator is selected from the group consisting of: 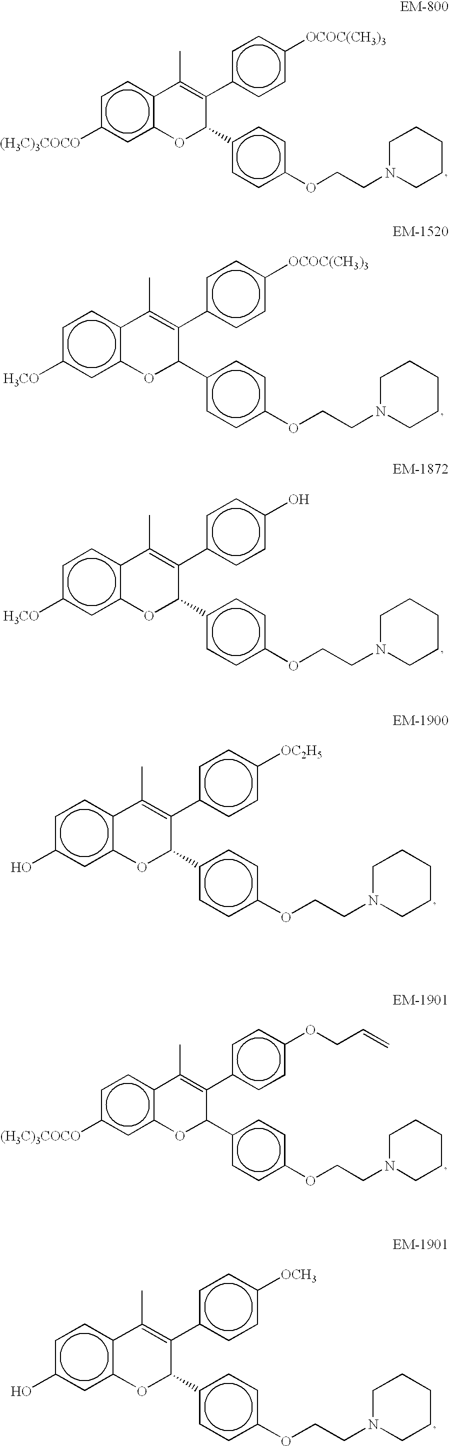
wherein all of the foregoing molecular structures whose stereochemistry is indicated are optically active due to a majority of their stereoisomers being of 2S configuration.

39. The kit of claim 36, wherein the benzopyran derivative is a salt of an acid selected from the group consisting of acetic acid, adipic acid, benzenesulfonic acid, benzoic acid, camphorsulfonic acid, citric acid, fumaric acid, hydroiodic acid, hydrobromic acid, hydrochloric acid, hydrochlorothiazide acid, hydroxy-naphthoic acid, lactic acid, maleic acid, methanesulfonic acid, methylsulfuric acid, 1,5-naphthalenedisulfonic acid, nitric acid, palmitic acid, pivalic acid, phosphoric acid, propionic acid, succinic acid, sulfuric acid, tartaric acid, terephthalic acid, p-toluenesulfonic acid, and valeric acid.

40. The kit of claim 39, wherein the acid is hydrochloric acid.

41. The kit of claim 22, wherein said selective estrogen receptor modulator is: 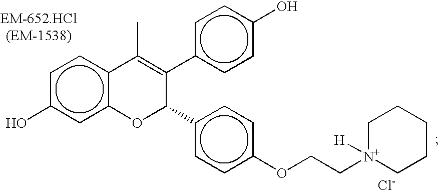
and is optically active due to a majority of its stereoisomers being of 2S configuration; and
wherein the estrogen is selected from the group consisting of 17β-estradiol, 17β-estradiol esters, 17α-estradiol, 17α-estradiol esters, estriol, estriol esters, estrone, estrone esters, conjugated estrogen, equilin, equilin esters, 17α-ethynylestradiol, 17α-ethynylestradiol esters, mestranol, and mestranol esters.

42. The kit of claim 22, wherein said estrogen is selected from the group consisting of 17β-estradiol, 17β-estradiol esters, estriol, estriol esters, estrone, estrone esters, conjugated estrogen, equilin, equilin esters, 17α-ethynylestradiol, 17α-ethynylestradiol esters, mestranol, mestranol esters, chemestrogen, DES, phytestrogen, tibolone, 2′-ethylestrogenoxazole, and ethynediol.

43. The kit claim 22, wherein said estrogen is a mixed estrogenic/androgenic compound.
